SEQUENCE LISTING (74 sequences)

SEQ ID NO: 1 - protein, 1226 aa, synthetic construct
MKKIDNFTNCYSLSKTLRFKAIPVGKTQENIDKKRLLEEDEKRAEDYKAVKKIIDRYHLSFINDVLNNVKLENLNEYASLFNKSNRDDSENKELEKLEMNMRKEIAKAFKNNEEYKKLFKKEIIEEILPEFLEDEEEKEIVNSFKGFTTAFTGFHENRENMYSDEEKSTSIAYRCINENLPRFISNIKIFEKVKAILDEDEIEEINEEILNNDYSVEDFFTVDFFNFVLTQEGIDVYNAIIGGIVTEDGTKIKGLNEYINLYNQKNKQRLPKLKPLYKQVLSERESMSFYAEGFTSDDEVLDALRNTLNKNSEIFNAIEKLKKLFSNLDDYNLDGIYVKNGPAITTISNDVFGEWSVIRDKWNEEYDLIHMKKKAKDTEKYEEKRRKEYKKIESFSIEELQELAGADLSIVEKIKEKISELIDEIKNAYSEAKNLFDADFTLEKKLKKDEKTVEIIKNLLDSVKDLEKYIKPFLGTGKESNRDEVFYGEFTPAFDAISEIDNLYNKVRNYVTQKPYSKDKFKLYFQNPQFMGGWDRNKETDYRATILRKNGKYYLAIMDKSNSKCLQNIPESENDNYEKMNYKLIPGPSKMLPKVFFSKKYMDYYNPSEEILRIYKNGTFKKGDSFNLNDCHKLIDFYKDSISRHPDWSKSFDFNFSETEKYKDISGFYREVDEQGYKVSFEKVSKSEVDTLVEEGKLYLFQIYNKDFSEKSHGTPNLHTMYFKALFDENNHGNIRLCGGAEMFMRRASIKKEELVVHPANQPIKNKNPDNPKKTTTLPYDVYKDKRFSEDQYELHIPISINKVPDNTFKINTEVRKLLRNDDNPYVIGIDRGERNLLYIVVVDGKGNIVEQYSLNEIINEYNGIKIRTDYHSLLDKKEKERLEARQNWKTIENIKELKEGYISQVVHKICELVEKYDAVIALEDLNSGFKNSRVKVEKQVYQKFEKMLIDKLNYMVDKKTDPSASGGVLNGYQLTNKFESFKKMGTQNGIMFYIPAWLTSKIDPTTGFVNLLKTKYTSIAEAKKFISSFDSIRYDSDEDMFEFSIDYNNFPRTDADYRKKWEIYSYGDRIRIFRNPKKNNEFDYETVNLTEKFKELFDKYGINYSSGDIREQLCAMSEKAFFEEFMGLLRLMLQMRNSITGRTDVDYLISPVKNSNGNFYDSRNYEKQESATLPKDADANGAYNIARKVLWAIEQFKKAEEDKLDKVKIAISNKEWLEYAQTHCK

SEQ ID NO: 2 - protein, 1224 aa, synthetic construct
MKKIDNFTNCYSVSKTLRFKAIPVGKTQENIDKKRLLEEDEKRAEDYKAVKKIIDRYHRSFIDKVLNNVKLDNLNEYASLFYKSNRDDSDNKKLEKLEAKMRKQIAKAFKNNEEYKKLFKKELIEEILPEFLEDEEEKEIVNSFKGFTTAFTGFHENRENMYSDEEKSTAIAYRCINENLPRFISNIKCFEKVKAILDEDEIEEINEEILNNDYSVEDFFTVDFFNFVLTQKGIDIYNAIIGGIVTEDGTKIQGLNEYINLYNQQNKQRLPQLKPLYKQVLSERESMSFYAEGFTSDDEVLDALRDTLGKNSTIFNAIEKLKKLFSNLDDYNLDGIYVKNGPAITTISNDVFGDWSVIRDKWNEEYDAVHSKKKAKDTEKYEEKRRKEYKKIESFSIAELQELVDSDNSIVEKIKEKIKELIDEIKNAYSEAKNLFDSDFKQEKKLKKDEKTVELIKNLLDSVKDLEKYLKPFMGTGKESNRDEVFYGEFTPCFDAISEIDNLYNKVRNYVTQKPYSTDKFKLYFQNPQFLGGWDRNKETDYRATILRKNGKYYLAIMDKSNSKVFQNIPESDDDNYEKMNYKLIPGPSKMLPKVFFSKKNIDYFNPSEEILRIYKNGTFKKGDSFNLDDCHKLIDYFKDSISKHPDWSKSFDFKFSETEKYKDISGFYREVDEQGYKVSFEKVSKSYVDTLVEEGKLYLFQIYNKDFSEKSHGTPNLHTMYFKALFDENNQGNIRLCGGAEMFMRRASIKKEELIVHPANQPIKNKNPLNPKKTTTLPYDVIKDKRFTEDQYELHIPITINKVPDNAFKINHEVRKLLRNDDNPYVIGIDRGERNLLYIVVIDGKGNIVEQYSLNEIINEYNGIKIRTDYHSLLDKKEKERLEARQNWKTIENIKELKEGYISQVVHKICELVEKYDAVIALEDLNSGFKNSRVKVEKQVYQKFEKMLIDKLNYLVDKKTDPSENGGVLHGYQLTNKFESFKKMGTQNGIMFYIPAWLTSKIDPTTGFVDLLKPKYTSIAEAKKFISSFDSIRYNSDEDMFEFSIDYNKFPRTDADYRKKWTIYTHGDRIRTFRNPKKNNEWDNETVNLTEKFKKLFEKYGINYSSGDLREQICAMSEKEFYKEFMGLLRLMLQMRNSITGRTDVDYLISPVKNSNGNFYDSRNYEKSATLPKDADANGAYNIARKVLWAIEQFKKAEDDKLDKVKIAISNKEWLEYAQTHCK

SEQ ID NO: 3 - protein, 1232 aa, synthetic construct
MKKIDNFTNCYSLSKTLRFKLIPVGKTQENIDKKRLLEEDEKRAEDYKAVKKIIDRYHRSFIDDVLSSVKLENLNEYAELFYKSNKSDSDKKKMEKLESKMRKQIAKAFKSNEGYKKLFKKELIKEILPEFLEDEEEREIVESFKGFTTAFTGFYENRKNMYSDEEKSTAIAYRCINENLPKFLDNVKCFEKVKAVLPKDEIEEINSDIQGLSGYSVEDVFSVDFFNFVLSQSGIDVYNAIIGGYTTEDGTKIQGLNEYINLYNQQVKKSHRLPKLKPLYKQILSDRESVSFYPEKFESDDEVLEAIRDTYSNNSAIYDTLEKLEKLFSNLDEYNTDGIYVKNGAAVTTISNSVFGDWSVIRDKWNEEYDSVHPKSKAKDTEKYEEKRKKAYKKIESFSIAELQKLADSSAKSIVEYFKSAIKELVDEIKEAYASAKDLLNSPYTEEKNLKKNDKAVELIKNLLDSVKELENFLKQFMGTGKESNKDEVFYGDFTPCYDKLSQIDKLYDKVRNYVTQKPYSTDKFKLYFENPQFLGGWDRNKETDYRAVLLRKDGQYYLAIMDKKHSRVFEKIPESDDEDCYEKMVYKLLPGPNKMLPKVFFSKKNIDTYNPPEEILKIYKKGTFKKGDSFNLDDCHKFIDFFKDSIEKHPDWSYFDFKFSDTEEYKDISDFYREVEEQGYSISFEKVSESYIDELVEEGKLYLFQIYNKDFSEHSKGTPNLHTMYFKMLFDENNLEDVVIKLNGGAEMFMRKASIKKDELIVHPANQPIKNKNPQNPKKQSTFEYDIIKDKRFTEDQYSLHIPITINKKAENATNINDEVRKLLKDCDDNYVIGIDRGERNLLYICVIDGNGKIVEQYSLNEIINEYNGIKYKTDYHKLLDKKEKERDEARKNWKTIENIKELKEGYISQVVHKICQLVEKYDAVIAMEDLNSGFKRSRVKVEKQVYQKFEKMLIDKLNYLVDKKTDPDETGGLLHGYQLTNKFESFKKMGTQNGFIFYVPAWLTSKIDPTTGFVNLLKPKYTSVEEAKEFISRFDSIRYNADEDFFEFDIDYNKFSRTDADYRKKWTLCSYGDRIRTFRNPEKNNQWDNKTVTLTEEFKELFEKYGIDYTSGDLKEQICSVSDADFYKKFMGLLRLTLQMRNSITGRTDVDYLISPVKNKNGTFYDSRNYDGQENATLPKDADANGAYNIARKALWAIEQIKKAEDDELNKVKIAISNKEWLEYAQTSKK

SEQ ID NO: 4 - protein, 1278 aa, synthetic construct
MSLNKFTNQYSLSKTLRFELKPIGKTLEHIQNKGLLSQDEQRAESYKKMKKTIDGFHKHFIELAMQNVKLTKLKEFADLYNASAERKKDDEYKKELEKIQAELRKEIAEGFKTGAAKEIFSKLDKKELITELLENWIRTQEDEDIYFDESFKTFTTYFGGFHENRKNMYTDKEQSTAIAYRLIHENLPKFLDNIRVFDKIKEIPELYEKLPLLYKEIKEYLNISSIDEAFSLDYFNKVLTQKQIDVYNLIIGGRTPEEGKKKIQGLNEYINLYNQQQKDKNNRIPKLKMLYKQILSDRESTSFLPEAFESSQEVLDAINSYYHSNLISFQPEDKEEAENVLEKIKDLLTHLKDYDLNKIYLRNDTQLTHISQKLFGNYAVLGDALSFYYDQVLAPSYQEDYQSANERKRKKLEKEKEKFLKQDYFSIAQLQNALDAYINSLDDTKDLKKNYTTNCIADYFHTHFKAEKKEDEDKEFDLIANIEAKYSCVKGILENYPKDRKLHQDKKTIDDIKLFLDSLMELLHFVKPLILPSDSALEKDEAFYGQLEPWYDQLELLIPLYNKVRNYATQKPYSTEKFKLNFENSTLLNGWDVNKESDNTSVIFRKDGNYYLGIMDKKHNKIFKNVPKASTGESTYEKMVYKLLPGPNKMLPKVFFSDKNINYFAPSEEIQKIRKHGTHKKGEDFNLNDCHKLIDFFKSSIEKHEDWKNFGFQFSDTATYDDLSEFYREVEHQGYKITFTDIDENYINQLVDEGKLYLFQIYNKDFSPYSKGRPNLHTLYWKALFDPENLKDVVYKLNGQAEVFYRKKSIKAENMVIHKAGEAIDNKNPLTTKKQSTFEYDLIKDKRYTVDKFQFHVPITLNFKASGKDNINQEVLEYLKNNPDVNIIGIDRGERHLIYLTLIDQKGNILKQESLNTIVSERYNIETNYHELLAKREKERDKARKNWGTIENIKELKEGYLSQVVHKIAKMMVEHNAIVVMEDLNFGFKRGRFKVEKQVYQKLEKMLIDKLNYLVFKDKSDNEPGGLYKALQLTNKFESFKELGKQSGFLFYVPAWNTSKIDPTTGFVNLFNTRYENIEKAREFFGKFESIRYNSDKGYFEFAFDYNDFTTKAEGTRTDWTVCTHGERIKTFRNPEKNNQWDNKEIDLTEEFKDLFKKYGITYGDGKDIREQITSQTDKAFYERLLHLFKLTLQMRNSKTGTDIDYLISPVMNAKGEFYDSRKADNTLPQDADANGAYHIAKKGLWLLQQINQAEDWKKLKLAISNKEWLRFVQGYKK

SEQ ID NO: 5 - protein, 1257 aa, synthetic construct
MKSFDEFTNLYSLSKTLRFELKPVGKTLEHIEKNGIIEQDEQRAESYKKVKKIIDEYHREFIEEALSNVSLTKDNLEEYAELYKKSKERKKDDKEKKELEEIQAELRKEIANCFKKNERFKNLFAKELITNELQSWVQDKEEINVIEKFNNFTTYFTGFHENRKNMYSDEDKSTAIAYRIIHENLPKFLDNIKVFQKIKEVYEKYELTLLEKELKSELGATSLDEIFSLDYFNKVLTQKGIDRYNLIIGGRTTEDGEKIQGLNELINLYNQQQKDKNKRIPKLKPLYKQILSDRESTSFIPEAFENDNEVLEAINDFYQEILIYRPEDKDKSENVLEKLKDLLQHLKDYDLDKIYIRNDKSITDISQKIFGDWSIIKSALKYYYDSKLATGKKKTTQKQEKEKEKWLKQDYFSIAEIEDALSAYKNNAEDSKLSENPIADYFKKFMKNEKSEEEKEQDLFANIEAKYSSIKDLLEKDYTKDKKLHQDKENVDKIKAFLDSIMELLHFVKPLILKSDSALEKDEAFYSEFEEFYEQLEQIIPLYNKVRNYMTQKPYSTEKFKLNFENSTLLNGWDANKESDNTSVILRKDGKYYLGIMNKKHNKIFKNVPKAGSEAYYEKMVYKLLPGPNKMLPKVFFSKKNIDYFKPSQEILKIRNSGSHKKGDDFNLEDCHKLIDFFKECIEKHPDWKNFDFQFSPTESYEDISEFYREVEHQGYKITFTKIPENYIDQLVDEGKLYLFQIYNKDFSPHSKGKPNLHTLYWKALFDPENLKDVVYKLNGEAEVFYRKKSIKKEEKIVHKAGKPIPNKNHHNSKKQSKFDYDIIKDRRYTEDKFLFHVPITLNFKAKGKNNINQEVNEFLKNNEDVHIIGIDRGERHLLYLSLINQKGNIIEQGSLNTITNEHYNYEVDYHEMLDKREKERDKARKNWKTIENIKELKEGYLSQVVHKIAKMMVEHNAIVVMEDLNFGFKRGRFKVEKQVYQKFEKMLIDKLNYLVFKDKKPNEPGGVLKAYQLTSKFESFKKLGKQSGFLFYVPAAYTSKIDPTTGFVNLLHPKYENIEKAKEFFNKFESIRYNSNKDYFEFSFDYNKFTTKAEGKKTDWTVCTHGERFKTVRNNNGQWDSKEVDVTEELKNLFNEYGINYEDGKDIKEQITKTTNKKFFKRLLHLLKLTLQMRHSNTDSEEDYILSPVKNEKGEFFDSRKADDTLPMDADANGAYHIALKGLLLLQQIKQAEDLKKLNLWISNKEWLQFVQNNKR

SEQ ID NO: 6 - protein, 1245 aa, synthetic construct
MKTFDDFTNLYSLSKTLRFELIPVGKTLEHIEKKGLIEEDEKRAENYQKVKKIIDRYHKYFIEQALNNVKLDDLEEYQTLYHKKKKDDNQKKEFEKIQEKLRKQIADAFKSNERFKKLFKKELIKELLPEFVQEEEERELVESFKNFTTYFTGFHENRKNMYSDEEKSTAIAYRLIHENLPKFLDNMKIFEKIKAAPPKEKIEELYKDLEEYLNVTSIEEVFSLDYFNEVLTQKGIDVYNTIIGGRTAEEGKTKIQGLNEYINLYNQQQKKNKRLPKLKPLYKQILSDRESTSFIVEQFENDQEVLEAIEEFYQELIASYEGKGETVNVLETLKELLSNLSEYDLDKIYLRNDKSLTDISQKIFGDWSVIQNALSEYYDKVIPGKKKKDTEKYEEKRKKKFKKQDYFSIAELQTALDTYEKEKYSTNSIVDYFATLGNESEKEFNLVEKIENAYSSVKDLLNTPYPEDKNLHQDKESVEKIKNFLDSIMDLLHFLKPLMATEETLEKDQTFYGEFEPLFEELSQIIPLYNKVRNYVTQKPYSTEKFKLNFENPTLLDGWDKNKETDNTGVLFRKDGQYYLGIMDKKHNRVFENIPEPNDDDCYEKMEYKLLPGPSKMLPKVFFSKSNIDYFNPSEEILRIYNHGTHKKGENFNLEDCHQLIDFFKESINKHPDWKNFGFKFSPTKQYESISEFYREVEEQGYKISFTKISESYIDQLVEEGKLYLFQIYNKDFSPHSKGKPNLHTLYWKALFDEENLKDVVYKLNGQAEVFYRKASIKKENKIVHPANQAIANKNPLNKKKQSVFEYDIIKDKRFTVDKFQFHVPITLNFKATGSDNINQEVNEYLRQNPDVHIIGIDRGERHLIYLTLIDQKGNIIEQESLNTITNEHHTIRTPYHELLDKKEKERDEARKSWKTIENIKELKEGYLSQVVHKIAKLMVKYNAIVVMEDLNFGFKRGRFKVEKQVYQKLEKMLIDKLNYLVFKDVEPDEPGGLLHALQLTNKFESFKKMGKQSGFLFYVPAWNTSKIDPVTGFVNLLHPKYENVEKAKEFFSKFDSIRYNPEKDYFEFAFDYNNFTTKAEGTRTKWTICTYGERIKTFRNPEKNNQWDNRTVNLTEEFKKLFEEYGIDYSNGGDLKEQICEQNDKDFFKKFMNLLKLTLQMRNSITGTEVDYLISPVANTQGEFFDSRNADESLPQDADANGAYHIALKGLWVIEQIKQADDLKKIKLAISNKEWLQFVQNRNK

SEQ ID NO: 7 - protein, 1349 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 8 - protein, 1527 aa, synthetic construct
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEESGGSSGGSSGSETPGTSESATPESSGGSSGGSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 9 - protein, 1527 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLSGGSSGGSSGSETPGTSESATPESSGGSSGGSEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 10 - protein, 1443 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLSGGSSGGSSGSETPGTSESATPESSGGSSGGSATVKFKYKGEEKEVDISKIKKVWRVGKMISFTYDEGGGKTGRGAVSEKDAPKELLQMLEKQKK

SEQ ID NO: 11 - protein, 1349 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGKIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 12 - protein, 1349 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVRKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 13 - protein, 1349 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGKIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVRKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 14 - protein, 1527 aa, synthetic construct
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEESGGSSGGSSGSETPGTSESATPESSGGSSGGSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVRKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 15 - protein, 1527 aa, synthetic construct
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEESGGSSGGSSGSETPGTSESATPESSGGSSGGSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGKIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 16 - protein, 1527 aa, synthetic construct
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEESGGSSGGSSGSETPGTSESATPESSGGSSGGSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFRKKTRTENKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGKIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVRKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 17 - protein, 1362 aa, synthetic construct
MSKVNNGFSSLCVWSDFTRKFSLSKTLRFELKPVGRTEDFLKQNKVFEKDKTIDDSYNQAKFYFDKLHQKFINESLSPSSDSSNLKNIDLEYFAKQFLKLKSEIQKLKGEKKQKEANNKEKEINNLRKAYYKEIKSLLDKKAEEWKEIYKKKEIQFNETDLKQKGTDFLMKSGILGILKYEFPKEKEEELKSKDWPSLFVEDKANPGDKVYIFDSFDDFATYLIKFQETRKNLYKDDGTSTAVATRVISNFEKFLNNKKIFKDKYINYWKQIGITEGEKQIFEIDYYYNCFIQSGIDNYNDLIGKINQKSKQYRDKNKIEKSKLPLFKVLDKQILGEVIKERELIIKTETETEEEVFINRFKEFIDQNKQRILRAQNLMNDLINEEFENEYSGIYLKNSAINTIANRWFKNTTEFLLKLPQASKSKENKESPKVEPFVSLLDIKNALDDFEKEKDSLGTIFKDKYYKTKESDEAPLNSDSQESYWKQFLKIWGYEFNQLFEDKFNEEGIKIFWGYTEDLEKQAENLNSFSRKKEEIILVKNYCDATLRIYQMMKYFALEAKKQDDIPLAADCSPEFYNRFDEYYKDFKFIRYYDAIRNFVTKKPSNEDKIKLNFESGSLLTGFDKNKESEKLGIILKNKNNNKYFLGIINKKHNKIFESKNENEFIGNPAKDLYEKMELKLFPDPKKMIPKIAFADKNKKDFGWTQEIQKIKEDFGNFQENKKDSKDLKFDKNKLSKLIEYYQNCLEKGNYKKEFDFEWKKPEEYQSMSEFNQDIEKKNYKIKFIPIKASYIDDKVKNGELYLFEISNKDFIWPNQKKNIHTLYFLNLFSDKNIQKPVFRLGANAEVFYRPASVRKEMDKERSKGGKEIIKYKRYTEDKMFLHLPIEINYGCPKAPNKNQYNKKIIEFLNKNKDEINIIGIDRGEKNLLYYTVINQKGEILDHGSLNEINGVNYFEKLIEREKERQINRQSWEPVVKIKDLKKGYLSYIVRKIADLVEKYNAIIVLEDLNMRFKQVRGGIERSIYQQFEKQLIDKLGYLVFKDDRGPESPGGVLNGYQLLAPFTTFKDLGKQTGIIFYTNAEYTSKTDPITGYRKNIYISNSASQKKIKENLINKLKEIGWDEKENSYFFTYNQKDFGSPISKEWTLYSKVPRVIKENNNSTGYWEYKPIDLNEEFEDLFKKYGINEKSSDILSEIKELIKNNEGKLTRKQEFDGKNKNFYERFVYLFNLLLETRNTMSLRVKLDKRGNEIKLDEIDYGVDFFASPVKPFFTTAGVRFVGRQIEGGKIQKEKKEEFTIKNFSDFERLFKNCPSDGFDSDGVGAYNIARKGIMILERIKQNPKNPDLYISKDDWDSFVLRNLS

SEQ ID NO: 18 - RNA, 40 nt, synthetic construct
  source 1..40: mol_type = other RNA
caatttctacttttgtagatggaagagacaggtacgttaa

SEQ ID NO: 19 - RNA, 40 nt, synthetic construct
  source 1..40: mol_type = other RNA
caatttctacttttgtagatttgctttcgtggtattcttg

SEQ ID NO: 20 - RNA, 40 nt, synthetic construct
  source 1..40: mol_type = other RNA
caatttctacttttgtagatcaagactcacgttaacaata

SEQ ID NO: 21 - RNA, 40 nt, synthetic construct
  source 1..40: mol_type = other RNA
caatttctacttttgtagatgtggtattcttgctagttac

SEQ ID NO: 22 - RNA, 40 nt, synthetic construct
  source 1..40: mol_type = other RNA
caatttctacttttgtagatgtggtattgttgctagttac

SEQ ID NO: 23 - RNA, 40 nt, synthetic construct
  source 1..40: mol_type = other RNA
caatttctacttttgtagatgtggtattcttgcttgttac

SEQ ID NO: 24 - RNA, 40 nt, synthetic construct
  source 1..40: mol_type = other RNA
caatttctacttttgtagatgtggtattgatgctagttac

SEQ ID NO: 25 - RNA, 40 nt, synthetic construct
  source 1..40: mol_type = other RNA
caatttctacttttgtagatgtcctattcttgcttgttac

SEQ ID NO: 26 - RNA, 40 nt, synthetic construct
  source 1..40: mol_type = other RNA
caatttctacttttgtagattgtgcaataattggactttg

SEQ ID NO: 27 - RNA, 40 nt, synthetic construct
  source 1..40: mol_type = other RNA
caatttctacttttgtagatatcagaaatgactccatgaa

SEQ ID NO: 28 - RNA, 40 nt, synthetic construct
  source 1..40: mol_type = other RNA
caatttctacttttgtagattactcaatcagctattgtca

SEQ ID NO: 29 - RNA, 40 nt, synthetic construct
  source 1..40: mol_type = other RNA
caatttctacttttgtagataatgatctccacgcaattgt

SEQ ID NO: 30 - RNA, 40 nt, synthetic construct
  source 1..40: mol_type = other RNA
caatttctacttttgtagataagactcttccagtgacaat

SEQ ID NO: 31 - RNA, 40 nt, synthetic construct
  source 1..40: mol_type = other RNA
caatttctacttttgtagattgtgcaatccttggactttg

SEQ ID NO: 32 - RNA, 111 nt, synthetic construct
  source 1..111: mol_type = other RNA
gtctagaggacagaatttttcaacgggtgtgccaatggccactttccaggtggcaaagcccgttgagcttctcaaatctgagaagtggcaccgaagaacgctgaagcgctg

SEQ ID NO: 33 - RNA, 40 nt, synthetic construct
  source 1..40: mol_type = other RNA
caatttctacttttgtagatcccccagcgcttcagcgttc

SEQ ID NO: 34 - DNA, 153 bp, synthetic construct
  source 1..153: mol_type = other DNA
ttcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaacct

SEQ ID NO: 35 - DNA, 19 bp, synthetic construct
  source 1..19: mol_type = other DNA
ttcgtttcggaagagacag

SEQ ID NO: 36 - DNA, 20 bp, synthetic construct
  source 1..20: mol_type = other DNA
aggttttacaagactcacgt

SEQ ID NO: 37 - DNA, 25 bp, synthetic construct
  source 1..25: mol_type = other DNA
atgtcgtcagtgtgtgtgtgtgacg

SEQ ID NO: 38 - DNA, 15 bp, synthetic construct
  source 1..15: mol_type = other DNA
ttttttttttttttt

SEQ ID NO: 39 - skipped

SEQ ID NO: 40 - RNA, 10 nt, synthetic construct
  source 1..10: mol_type = other RNA
cccccccccc

SEQ ID NO: 41 - DNA, 20 bp, synthetic construct
  source 1..20: mol_type = other DNA
gaattgatgcaatggagcta

SEQ ID NO: 42 - DNA, 20 bp, synthetic construct
  source 1..20: mol_type = other DNA
gcagcataagtagtatgtcg

SEQ ID NO: 43 - DNA, 42 bp, synthetic construct
  source 1..42: mol_type = other DNA
tctccacgcaattgtcgatattggtagcgagttgaaggagtt

SEQ ID NO: 44 - DNA, 39 bp, synthetic construct
  source 1..39: mol_type = other DNA
actccatgaaaaccgccaaagaagactcttccagtgaca

SEQ ID NO: 45 - DNA, 19 bp, synthetic construct
  source 1..19: mol_type = other DNA
ccacggaagtgaattcgag

SEQ ID NO: 46 - DNA, 22 bp, synthetic construct
  source 1..22: mol_type = other DNA
tggactttgtactcaatcagct

SEQ ID NO: 47 - DNA, 450 bp, synthetic construct
  source 1..450: mol_type = other DNA
atggcctctccttgtgcccagttcagtccctgtcattgccacgctactaaggactccctgaataccgtgactgacgtcagacattgtctgactgaatacatcctgtgggtttctcatagatggacccatagagaaagcgcagggcctctctacaggcttctcatctctttcagaattgatgcaatggagctatttggtagcgagttgaaggagttctcgaattcacttccgtgggacaatatcgacaattgcgtggagatcattaaatgtttcatcagaaatgactccatgaaaaccgccaaagaactttgtgcaataattggactttgtactcaatcagctattgtcactggaagagtcttcaatgataagtatatcgacatactacttatgctgcgaaagattctgaacgagaacgactatctcaccctcttggatcatatcctcact

SEQ ID NO: 48 - DNA, 450 bp, synthetic construct
  source 1..450: mol_type = other DNA
atggcctttccttgtgcccagttcagtccctgtcattgccacgctactaaggactccctgaataccgtgactgacgtcagacattgtctgactgaatacatcctgtgggtttctcatagatggacccatagagaaagcgcagggcctctctacaggcttctcatctctttcagaactgatgcaatggagctctttggtagcgagttgaaggagttctcggattcacttccgtgggacaatatcgacaattgcgtggagatcattaaatgtttcatcagaaatgactccatgaaaaccgccaaagaactttgtgcaatcattggactttgtactcaattagctattgtctctggaagagtcttcaatgataagtatatcgacatactacttatgctgcgaaagattctgaatgagaacgactatctcaccctcttggatcatatcctcact

SEQ ID NO: 49 - DNA, 18 bp, synthetic construct
  source 1..18: mol_type = other DNA
gctgctgaggcttctaag

SEQ ID NO: 50 - DNA, 21 bp, synthetic construct
  source 1..21: mol_type = other DNA
gcgtcaatatgcttattcagc

SEQ ID NO: 51 - DNA, 40 bp, synthetic construct
  source 1..40: mol_type = other DNA
gcggccaatgtttgtaatcagtagacgtggtccagaacaa

SEQ ID NO: 52 - DNA, 39 bp, synthetic construct
  source 1..39: mol_type = other DNA
tcagcgttcttcggaatgtcgctgtgtaggtcaaccacg

SEQ ID NO: 53 - DNA, 22 bp, synthetic construct
  source 1..22: mol_type = other DNA
ccttgtctgattagttcctggt

SEQ ID NO: 54 - DNA, 18 bp, synthetic construct
  source 1..18: mol_type = other DNA
tggcatggaagtcacacc

SEQ ID NO: 55 - DNA, 21 bp, synthetic construct
  source 1..21: mol_type = other DNA
accaggaactaatcagacaag

SEQ ID NO: 56 - DNA, 25 bp, synthetic construct
  source 1..25: mol_type = other DNA
gacttgatctttgaaatttggatct

SEQ ID NO: 57 - DNA, 42 bp, synthetic construct
  source 1..42: mol_type = other DNA
ttccgaagaacgctgaagcggaactgattacaaacattggcc

SEQ ID NO: 58 - DNA, 40 bp, synthetic construct
  source 1..40: mol_type = other DNA
cgcattggcatggaagtcacaatttgatggcacctgtgta

SEQ ID NO: 59 - DNA, 21 bp, synthetic construct
  source 1..21: mol_type = other DNA
gggggcaaattgtgcaatttg

SEQ ID NO: 60 - DNA, 20 bp, synthetic construct
  source 1..20: mol_type = other DNA
cttcgggaacgtggttgacc

SEQ ID NO: 61 - DNA, 23 bp, synthetic construct
  source 1..23: mol_type = other DNA
tgagtacgaacttatgtactcat

SEQ ID NO: 62 - DNA, 22 bp, synthetic construct
  source 1..22: mol_type = other DNA
ttcagatttttaacacgagagt

SEQ ID NO: 63 - DNA, 42 bp, synthetic construct
  source 1..42: mol_type = other DNA
accacgaaagcaagaaaaagaagttcgtttcggaagagacag

SEQ ID NO: 64 - DNA, 44 bp, synthetic construct
  source 1..44: mol_type = other DNA
ttgctagttacactagccatccttaggttttacaagactcacgt

SEQ ID NO: 65 - DNA, 18 bp, synthetic construct
  source 1..18: mol_type = other DNA
cgctattaactattaacg

SEQ ID NO: 66 - DNA, 19 bp, synthetic construct
  source 1..19: mol_type = other DNA
gcgcttcgattgtgtgcgt

SEQ ID NO: 67 - DNA, 46 bp, synthetic construct
  source 1..46: mol_type = other DNA
accgcatcgaatgcatgtgagtcaaaatttcggaagagacaggtac

SEQ ID NO: 68 - DNA, 23 bp, synthetic construct
  source 1..23: mol_type = other DNA
tgagtacgaacttatgtactcat

SEQ ID NO: 69 - DNA, 21 bp, synthetic construct
  source 1..21: mol_type = other DNA
taacaatattgcagcagtacg

SEQ ID NO: 70 - DNA, 45 bp, synthetic construct
  source 1..45: mol_type = other DNA
ggattccgctccagacttgagtcaaaacaatcgaagcgcagtaag

SEQ ID NO: 71 - protein, 63 aa, synthetic construct
ATVKFKYKGEEKEVDISKIKKVWRVGKMISFTYDEGGGKTGRGAVSEKDAPKELLQMLEKQKK

SEQ ID NO: 72 - protein, 148 aa, synthetic construct
MEEKVGNLKPNMESVNVTVRVLEASEARQIQTKNGVRTISEAIVGDETGRVKLTLWGKHAGSIKEGQVVKIENAWTTAFKGQVQLNAGSKTKIAEASEDGFPESSQIPENTPTAPQQMRGGGRGFRGGGRRYGRRGGRRQENEEGEEE

SEQ ID NO: 73 - protein, 32 aa, synthetic construct
SGGSSGGSSGSETPGTSESATPESSGGSSGGS

SEQ ID NO: 74 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

(19) United States
(12) Patent Application Publication
(10) Pub. No.: US 2026/0092310 A1
(43) Pub. Date: Apr. 2, 2026

(54) CAS12A COMPOSITIONS AND METHODS

(71) Applicant: New England Biolabs, Inc., Ipswich, MA (US)

(72) Inventors: Juan Pan, Beverly, MA (US); G. Brett Robb, Somerville, MA (US); Nathan Tanner, West Newbury, MA (US); Ivan R. Correa, JR., Hamilton, MA (US)

(73) Assignee: New England Biolabs, Inc., Ipswich, MA (US)

(21) Appl. No.: 19/341,788

(22) Filed: Sep. 26, 2025

Related U.S. Application Data
(60) Provisional application No. 63/700,512, filed on Sep. 27, 2024.

(51) Int. Cl. C12Q 1/6823; C12N 9/22; C12N 15/11; C12Q 1/34; C12Q 1/48; C12Q 1/6806; C12Q 1/6844

(52) U.S. Cl. CPC C12Q 1/6823; C12N 9/226; C12N 15/11; C12Q 1/34; C12Q 1/485; C12Q 1/6806; C12Q 1/6844; C12N 2310/20; G01N 2333/9126; G01N 2333/922; G01N 2333/924

(57) ABSTRACT

The present disclosure relates, in some embodiments to compositions comprising a variant Cas12a and one-pot methods of using a variant Cas12a with amplification to detect a sequence of interest.

### CROSS-REFERENCE TO RELATED APPLICATION

This application claims priority to U.S. Provisional Application No. 63/700,512 filed Sep. 27, 2024, the entire contents of which are hereby incorporated in their entirety by reference.

### SEQUENCE LISTING STATEMENT

This disclosure includes a Sequence Listing submitted electronically in .xml format under the file name “NEB-492.xml” created on Sep. 27, 2024, and having a size of 90,956 bytes. This Sequence Listing is incorporated herein in its entirety by this reference.

### BACKGROUND

Nucleic acids are important features of biological agents (e.g., infectious agents). The ability to rapidly detect nucleic acids with high specificity and sensitivity is important in monitoring these agents. Methods to detect nucleic acids often must balance two or more of speed, sensitivity, specificity, and simplicity. Additional considerations (e.g., in repeated testing settings) may include ease of implementation and prevention of cross-contamination. Coupling isothermal amplification with Cas12a can improve nucleic acid detection that is based on isothermal amplification only.

### SUMMARY

Provided herein are Cas12a nucleases (e.g., variant Cas12a nucleases) comprising an amino acid sequence selected from: an amino acid sequence that is at least 80% identical (e.g., ≥85%, ≥90%, ≥92%, ≥94%, ≥95%, ≥97%, ≥98% identical) to an amino acid sequence selected from: SEQ ID NOS: 1-16. Also provided are methods employing the described Cas12a nucleases in one-pot nucleic acid detection coupling isothermal amplification methods. The methods involve incubating a reaction mixture containing (i) a DNA polymerase; (ii) a target nucleic acid; (iii) dNTPs; (iv) one or more primers, (v) a Cas12a nuclease, (vi) a Cas12a guide RNA, and (vii) optionally, a trans nucleic acid substrate under conditions suitable both for polynucleotide extension of the target nucleic acid to produce copied DNA product and for Cas12a nuclease activation for trans nuclease cleavage of the trans substrate. Further provided are compositions containing such a Cas 12a nuclease, as well as kits containing such a Cas12a nuclease and one or more components for carrying out nucleic acid detection. In some embodiments, methods further involve incubating the reaction mixture above with one or more components such as a reverse transcriptase, a DNA binding protein, a pyrophosphatase, and a helicase.

Cas12a nucleases (e.g., variant Cas12a nucleases) described herein have enhanced functionality, such as thermal stability and high trans nuclease activity. Enhanced functionality includes support for use in a wide range of isothermal amplification methods including, for example, high temperature methods (e.g., performed at 50-70° C.) such as loop-mediate isothermal amplification (LAMP) and strand-displacement amplification (SDA) as well as moderate temperature applications (e.g., performed at 34-42° C.) such as recombinase polymerase amplification (RPA). In some embodiments, disclosed Cas12a nucleases (e.g., variant Cas12a nucleases) may be used in nucleic acid detection applications comprising, for example, SDA amplification and detection reactions performed at the same temperature or comprising, for example, RPA amplification and detection reactions performed at the same temperature.

The present disclosure relates, in some embodiments to variant Cas 12a enzymes (e.g., thermostable variant Cas12a enzymes) and compositions comprising such enzymes. For example, a variant Cas 12a having an amino acid sequence at least 80% identical (e.g., ≥85%, ≥90%, ≥92%, ≥94%, ≥95%, ≥97%, ≥98% identical) to one or more of SEQ ID NOS: 1-16, wherein the variant Cas12a has thermostable cis recognition activity and thermostable trans nuclease activity. A composition comprising a variant Cas 12a may further comprise a crRNA operable with the variant Cas 12a and/or optionally, a non-naturally occurring buffer. In some embodiments, the molar ratio of variant Cas12a: crRNA is 1:1 to 1:20. A thermostable variant Cas12a may retain ≥85% of its peak activity at or upon or after exposure to a temperature of ≥55° C. for ≥5 minutes. In some embodiments, a variant Cas12a enzyme and/or a composition comprising a variant Cas12a enzyme may be cell-free and//or may lack catalytic activity beyond the enzyme's cis recognition activity and thermostable trans nuclease activity. A composition may further comprise a trans nuclease substrate (e.g., a trans nuclease substrate having a fluorophore, a linker polynucleotide, and a quencher, wherein the fluorophore and the quencher are each operably linked to the linker polynucleotide and wherein the quencher is operable to quench the fluorophore). According to some embodiments, a trans nuclease substrate may comprise a linker polynucleotide disposed between a fluorophore and a quencher, wherein the polynucleotide (e.g., DNA or RNA) is susceptible to trans nuclease cleavage (e.g., accessible to the variant Cas12a). A variant Cas12a enzyme and/or a composition comprising a variant Cas12a enzyme, according to some embodiments, may have any desired form including, for example, a dried, freeze dried, lyophilized, crystalline, or aqueous form. In some embodiments, a variant Cas12a may be immobilized on, to or upon a support, for example, with or without a linker disposed between the enzyme and the support. Compositions, in some embodiments, may comprise one or more additional components including, for example, a DNA polymerase, a glycosylase (e.g., a uracil DNA glycosylase), a nicking enzyme, a ligase, a helicase, a recombinase, a crowding agent, a DNA binding protein, a dye, an additive, a ribonucleoprotein, and/or combinations thereof. A DNA polymerase may be selected in accordance with desired properties. Examples of DNA polymerases include a Bst DNA polymerase, Bsu DNA polymerase, phi29 DNA polymerase, phi29-XT DNA polymerase, and/or variants thereof. The present disclosure provides, in some embodiments, kits (e.g., cell-free kits) including a non-naturally occurring buffer and a variant Cas 12a having an amino acid sequence at least 80% identical (e.g., ≥85%, ≥90%, ≥92%, ≥94%, ≥95%, ≥97%, ≥98% identical) to one or more of SEQ ID NOS: 1-16, wherein the variant Cas12a has thermostable cis recognition activity and thermostable trans nuclease activity

The present disclosure relates, in some embodiments, to methods of detecting a nucleic acid of interest. For example, a one-pot method may comprise contacting (e.g., reacting) at a single temperature and in a single container: (a) a variant Cas12a having an amino acid sequence at least 80% identical (e.g., ≥85%, ≥90%, ≥92%, ≥94%, ≥95%, ≥97%, ≥98% identical) to one or more of SEQ ID NOS: 1-16, wherein the variant Cas12a has thermostable cis recognition activity and thermostable trans nuclease activity; (b) a crRNA operable with the variant Cas12a (e.g., wherein the molar ratio of variant Cas12a: crRNA is optionally 1:1 to 1:20); (c) a polynucleotide comprising a nucleic acid sequence of interest; (d) optionally, amplification primers complementary to at least a portion of the sequence of interest and operable to support amplification of the sequence of interest; (c) optionally, a reverse transcriptase; (f) a DNA polymerase; (g) a trans nuclease substrate cleavable by the thermostable trans nuclease activity of the variant Cas12a; and (h) optionally, a non-naturally occurring buffer. According to some embodiments, the variant Cas12a and the crRNA are contacted in a single container under conditions that permit loop mediated amplification of the sequence of interest and trans nuclease cleavage of the trans nuclease substrate to produce a detectable trans nuclease cleavage marker. A one-pot method may be performed at a single temperature in any desired range, for example, a range of 20-30° C., 20-37° C., 34-38° C., 34-42° C., 34-65° C., 37-45° C., 37-55° C., 20-65° C., 55-65° C., or 50-60° C., according to some embodiments. In some embodiments, the amplification primers and the polynucleotide comprising the nucleic acid sequence of interest hybridize to form an amplification substrate for the DNA polymerase. A method may include, according to some embodiments, amplifying, by the DNA polymerase, the amplification substrate to produce an amplification product. Amplifying the amplification substrate may comprise, genome exponential amplification reaction (GEAR), helicase-dependent amplification (HDA), loop-mediated isothermal amplification (LAMP), multiple displacement amplification (MDA), nicking enzyme amplification reaction (NEAR), nucleic acid sequence-based amplification (NASBA), ramification (RAM), recombinase polymerase amplification (RPA), rolling circle amplification (RCA), self-sustained sequence replication (3SR), single primer isothermal amplification (SPIA), strand displacement amplification (SDA), transcription mediated amplification (TMA), or combinations thereof. In some embodiments, the crRNA hybridizes to the polynucleotide comprising the nucleic acid sequence of interest, the amplification product, or combinations thereof to form a cleavage assembly comprising the crRNA, the polynucleotide, and the variant Cas12a. A method may comprise, according to some embodiments, cleaving, by the variant Cas 12a, the polynucleotide comprising the nucleic acid sequence of interest (e.g., cis cleavage) and optionally cleaving by the variant Cas 12a, the trans nuclease substrate to form a trans nuclease cleavage product. In some embodiments, a method may comprise detecting (e.g., optically detecting the trans nuclease cleavage product and/or electrochemically detecting) a trans nuclease cleavage product. Examples of detecting a trans nuclease cleavage product include detecting with a sensor device. In some embodiments, the molar ratio of variant Cas12a: crRNA is 1:1 to 1:20.

### BRIEF DESCRIPTION OF THE FIGURES

FIG. 1 shows an example workflow of one-pot nucleic acid detection by coupling an amplification reaction and a Cas12a reaction in a single mixture at a single temperature. In this example, a sample to be interrogated is added to a master mix comprising reagents required for both amplification and a Cas 12a detection reaction. The mixture is then incubated at a single temperature for both reactions. Without necessarily limiting any embodiment to any particular mode of action, if the nucleic acid sequence of interest is present (illustrated in the upper track), Cas12a-crRNA ribonuclear protein complex, which has complementary spacer on the crRNA to the target nucleic acid sequence, would recognize the cis target. Recognition of a cis target triggers the Cas12a nonspecific nuclease activity on the reporter in trans. The reporters carry a fluorophore and a quencher which is spatially disposed to repress the fluorescent signal in the intact reporter. Fluorescence is rescued upon the trans cleavage of the DNA portion of the reporter by Cas12a. The reporter fluorescence signal thus is indicative of the presence of the cis substrate. If the nucleic acid sequence of interest is absent (illustrated in the lower track), the reporter remains intact (with the quencher and fluorophore in spatial proximity sufficient to suppress fluorescence) such that nominal or no signal is produced. The components for amplification and Cas12a-based nuclease reactions are present in the same reaction mixture, and once the testing sample is added, the reaction mixture is incubated at the same temperature to allow for tandem amplification, Cas 12a cleavage, and signal detection without disturbance or opening of the reaction vessel. This workflow enables easy-to-perform, rapid, sensitive, and specific one-pot nucleic acid detection.

FIG. 2 shows example results of a thermostability test of Cas 12a by heat pre-treatment. Each Cas12a was loaded with crRNA1 of SARS-COV-2 E gene to form an RNA-protein complex (RNP), then incubated at 55° C. for 5 minutes before cooling down to 4° C. At 4° C., each reaction was then added cis substrate E gene DNA (E1-PCR, generated from PCR amplification product of a synthetic gBlock of E gene), and trans substrate NZ-GT reporter. Reactions were then incubated at 55° C. The trans nuclease activity was monitored by FAM signal from cleavage of the NZ-GT reporter, which has a FAM fluorophore on the 5′ end, an internal quencher and another quencher on the 3′ end. Reactions were carried out in 1× modified NEBuffer r2.1 supplemented with 50 mM NaCl. FAM signal was monitored every 15 s in the SYBR channel. The presence of fluorescence with Cas12a variants Cas12a-JP16 and Cas 12a-JP1 as compared to absence of fluorescence with wild type LbaCas12a after the heat pre-treatment indicated that Cas12a-JP16 and Cas12a-JP1 are thermostable.

FIG. 3 shows example results of a trans nuclease activity assay using Cas12a-JP16. Four Cas 12a guides were designed on SARS-COV-2 E gene, and each guide (crRNA1-crRNA4) was loaded with Cas12a-JP16 to form an RNA-protein complex (RNP). The Cas12a RNP would bind and cleave cis substrate E gene DNA (E1-PCR, generated from PCR amplification product of a synthetic gBlock of E gene), which triggers the trans nuclease activity of Cas12a. The trans nuclease activity of Cas12a-JP16 was monitored by FAM signal from cleavage of the NZ-GT reporter, which has a FAM fluorophore on the 5′ end, an internal quencher and another quencher on the 3′ end. Reactions were carried out in 1× modified NEBuffer r2.1 supplemented with 0.8 mM dNTPs and 50 mM NaCl, and incubated at 55° C. FAM signal was monitored every 15 s in the SYBR channel. The efficiency of trans nuclease activity, estimated by the rate of relative fluorescence unit (RFU) change in the initial linear phase, varies for different guides. Guide crRNA4 was the most efficient under the assay conditions.

FIGS. 4A and 4B show example results of a one-pot reaction coupling Cas12a-JP16 and RT-LAMP using E gene crRNA2 (FIG. 4A) or crRNA4 (FIG. 4B). Traces show FAM fluorescence that arises from Cas12a-JP16 trans cleavage of the NZ-GT reporter after activation by the RT-LAMP reaction product. The upper panels of each of FIGS. 4A and 4B show fluorescence signal from reactions with template (positive, 200 copies/μl synthetic SARS-COV-2 RNA per reaction). The lower panels of each of FIGS. 4A and 4B show reactions with no-template control (NTC). The RT-LAMP Cas12a-JP16 one-pot reaction comprises RT-LAMP and Cas12-JP16a reagents in a single reaction mixture, with Cas12a-JP16 and crRNA2 (FIG. 4A) or crRNA4 (FIG. 4B) targeting E gene of SARS-COV-2. Each cycle was approximately 52 s.

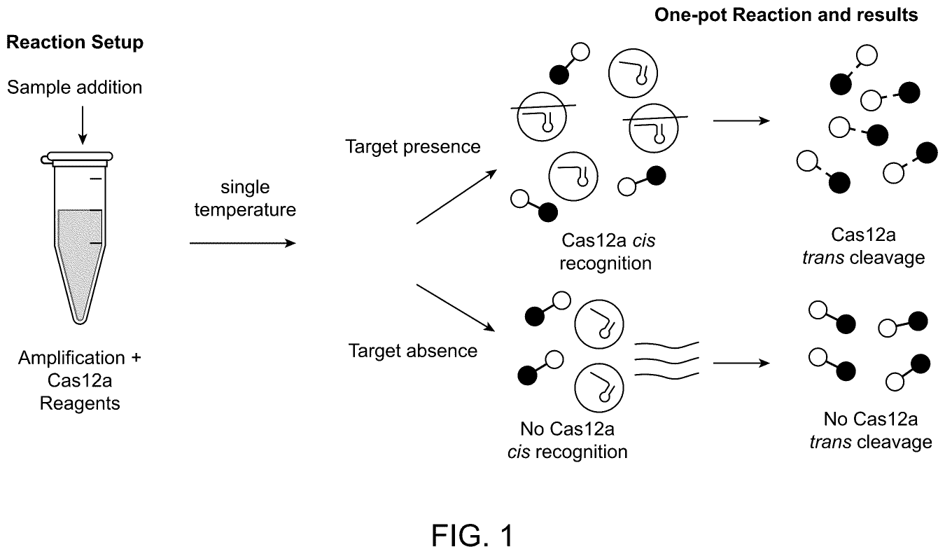

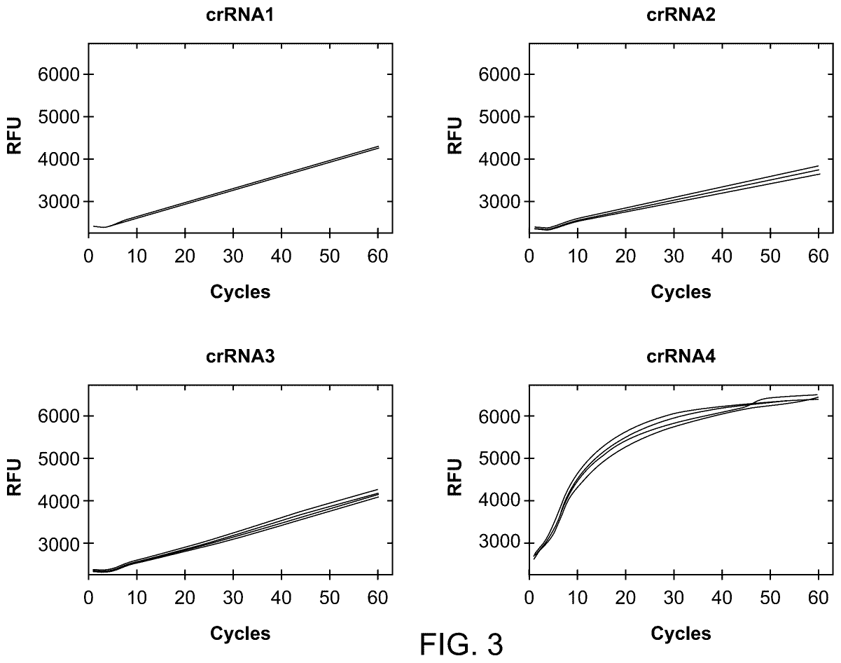

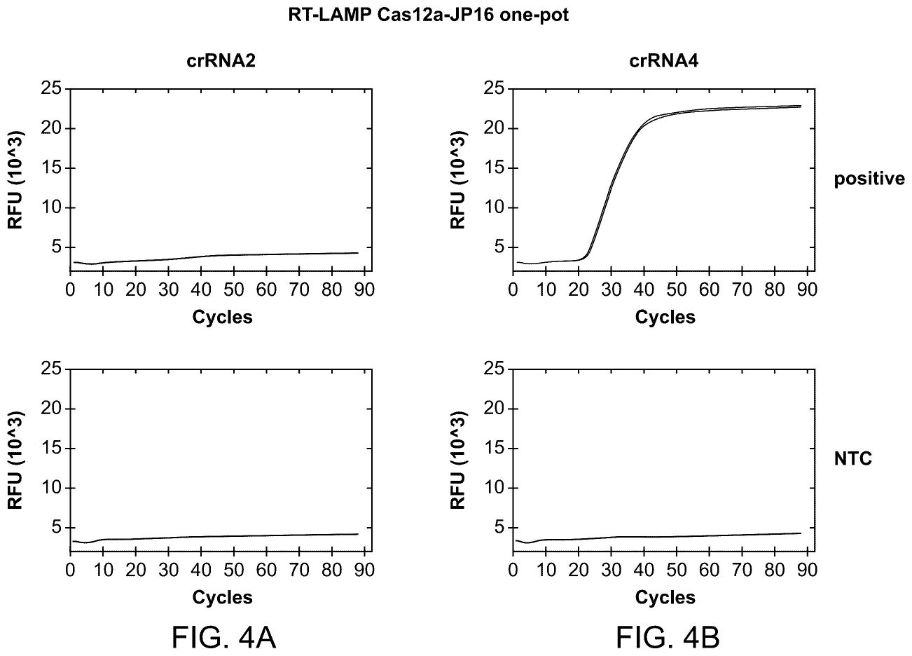

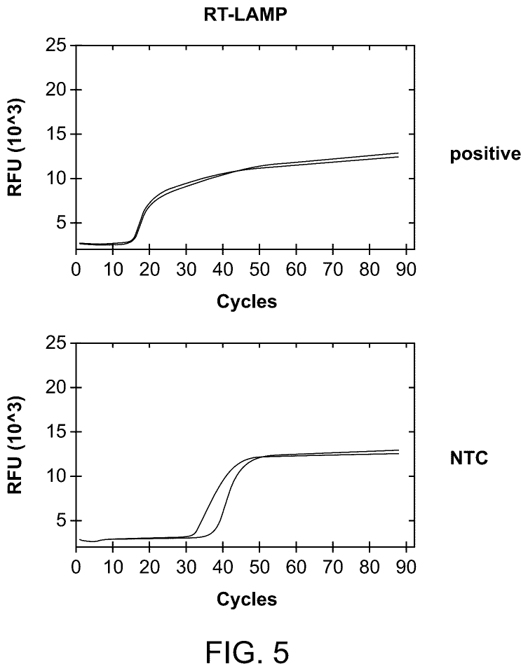

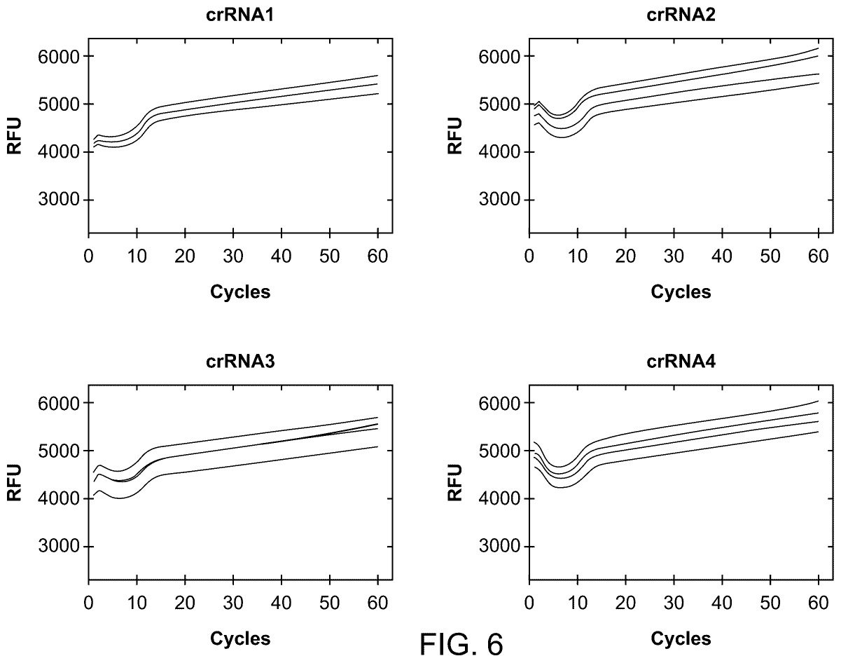

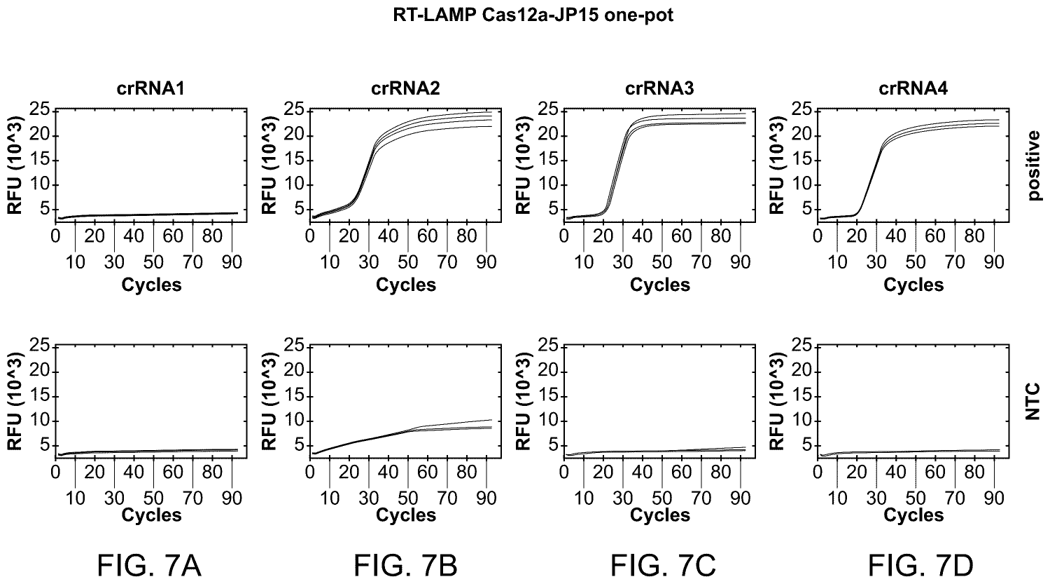

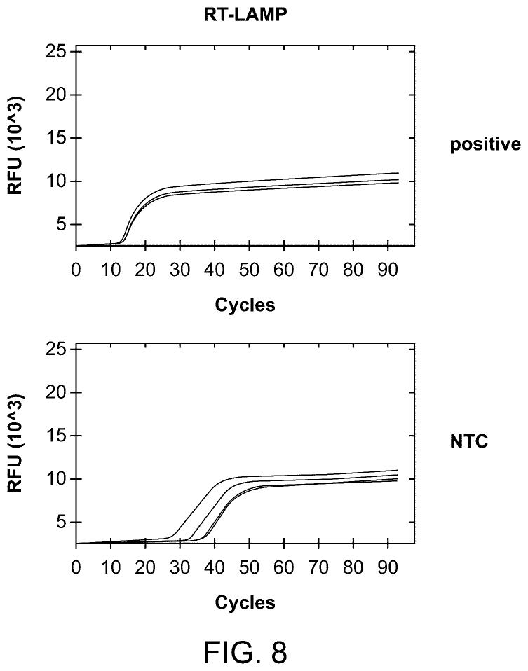

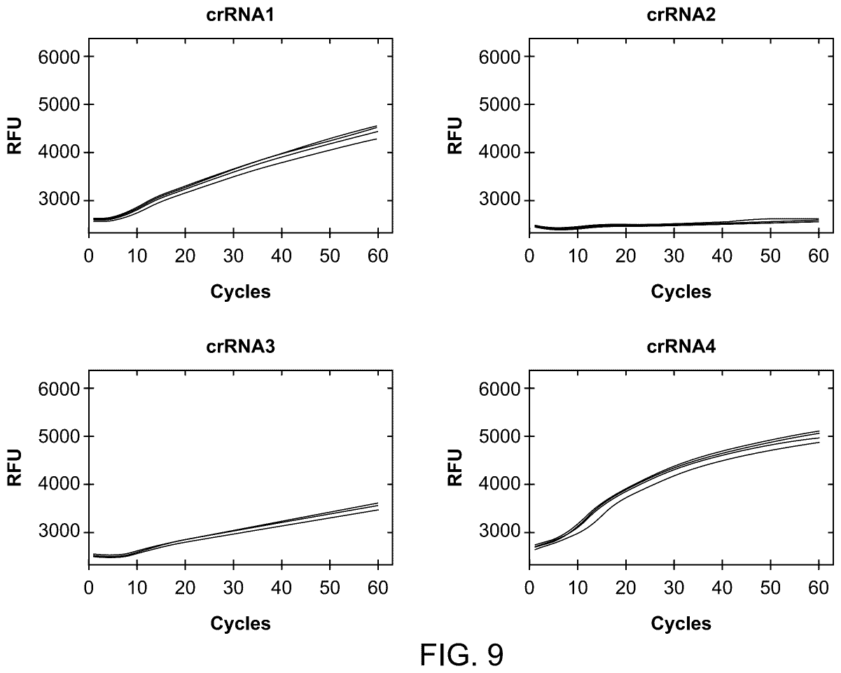

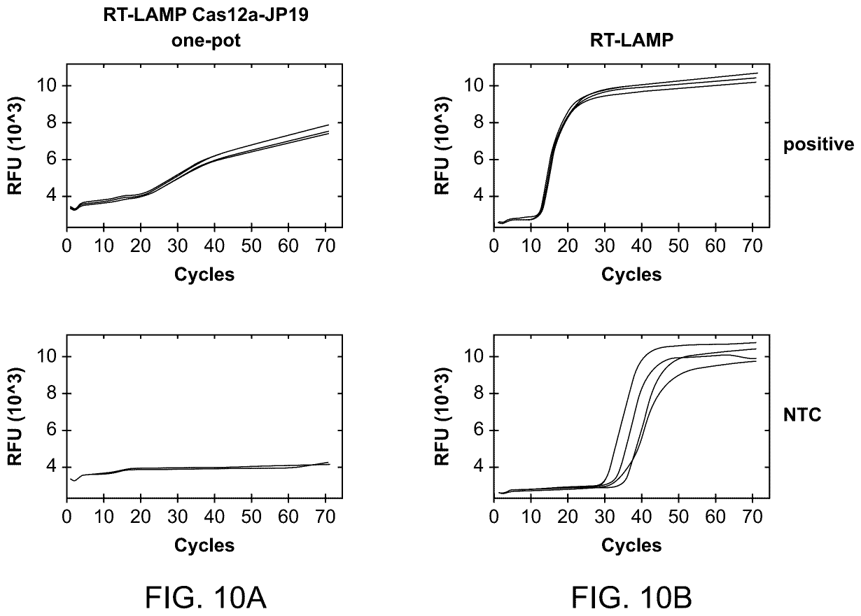

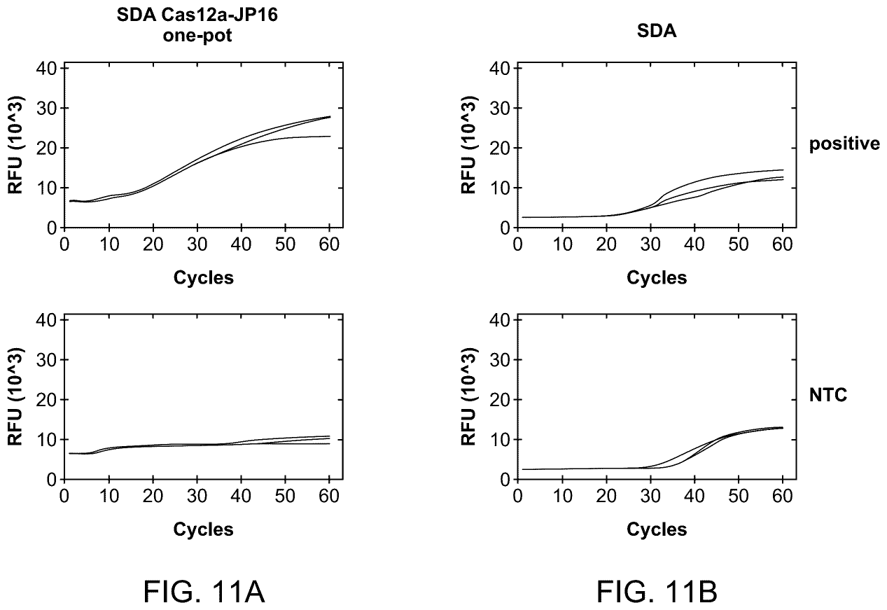

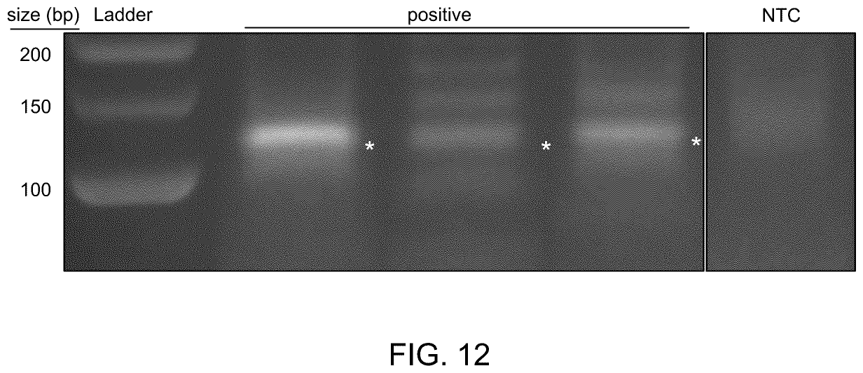

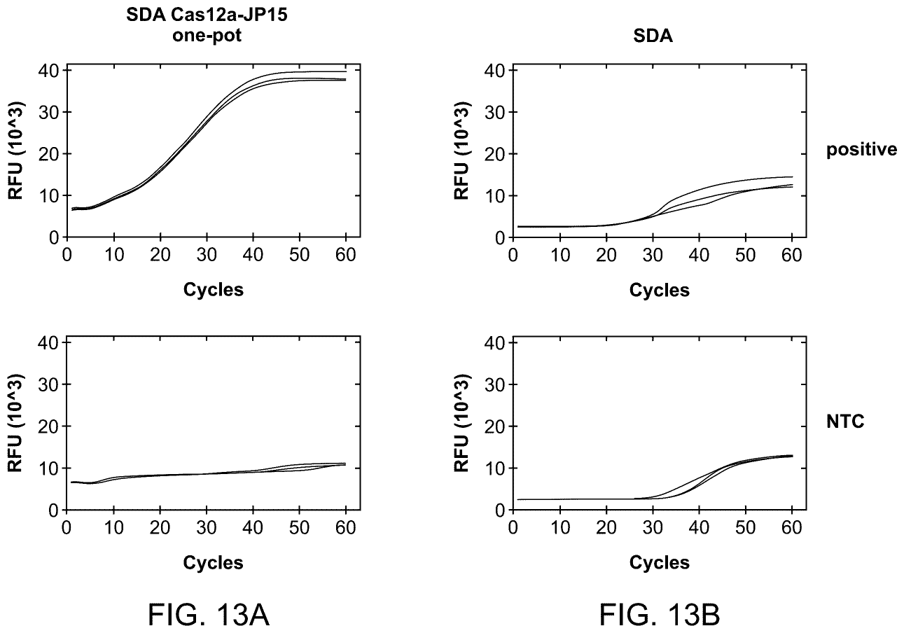

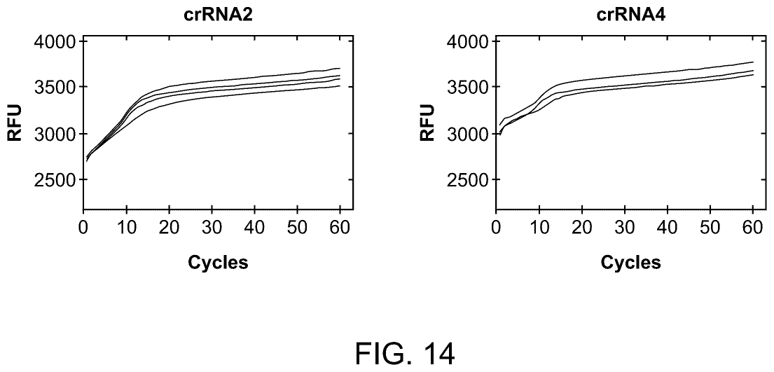

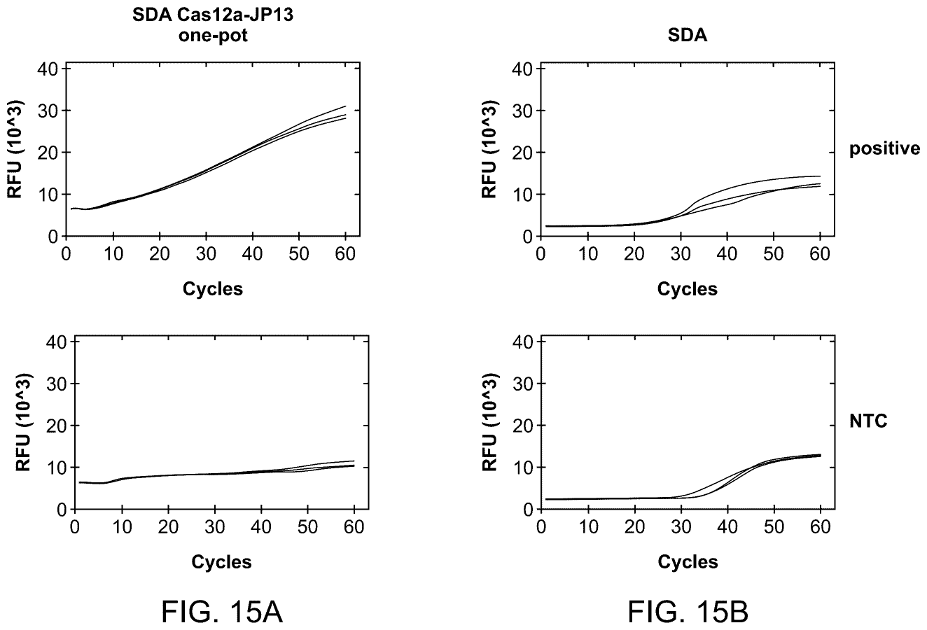

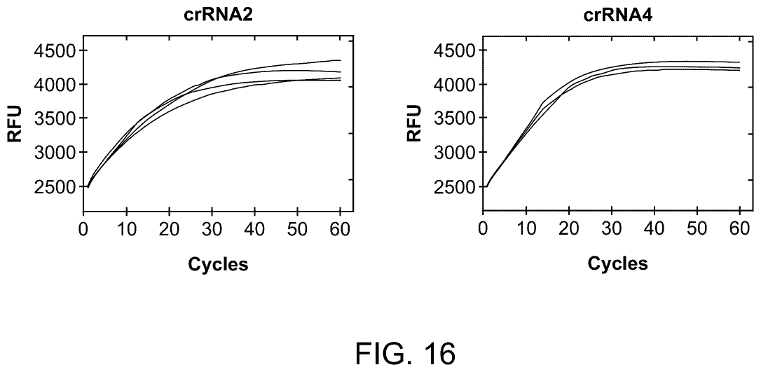

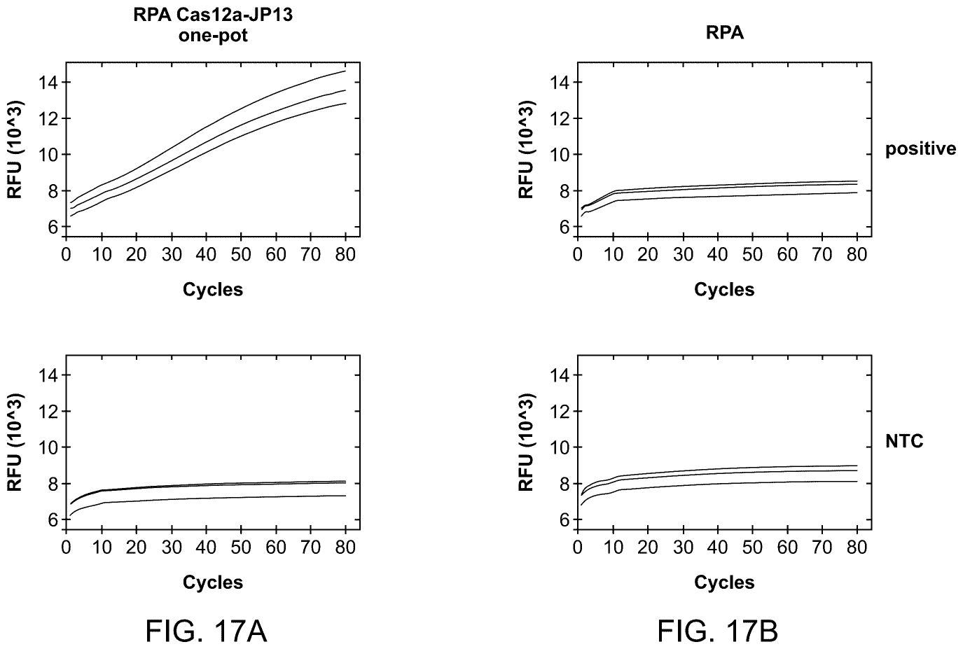

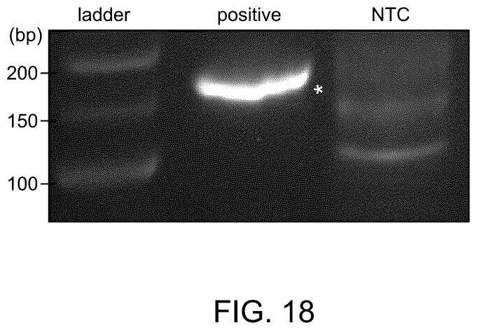

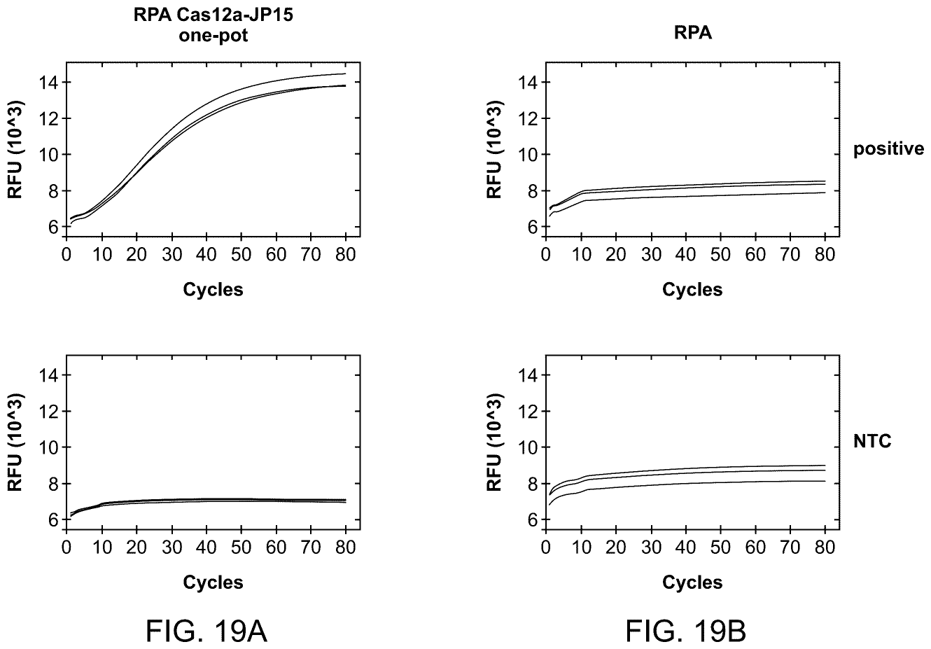

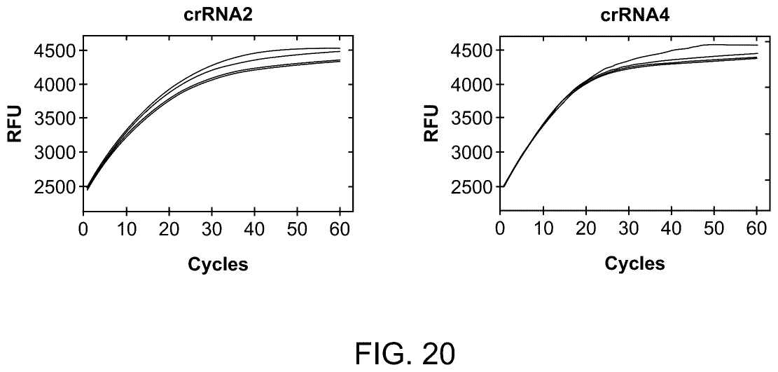

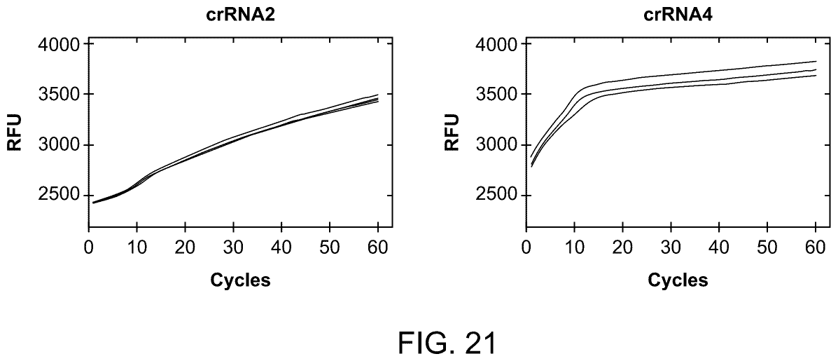

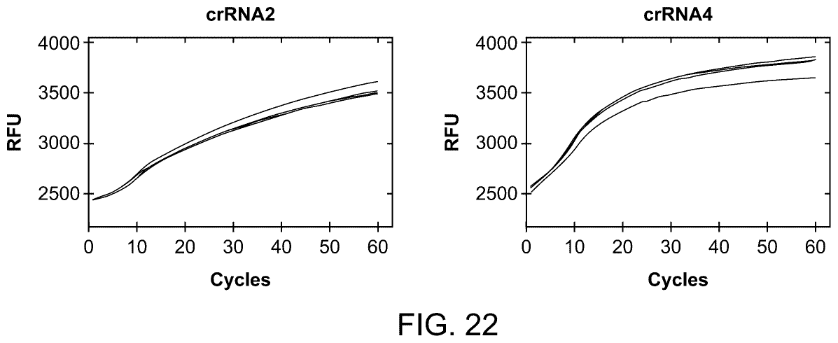

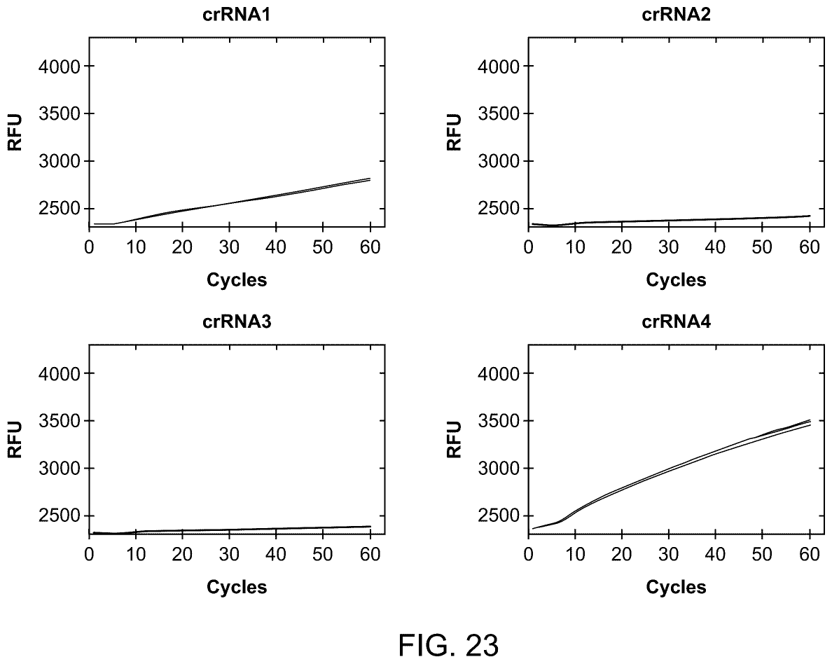

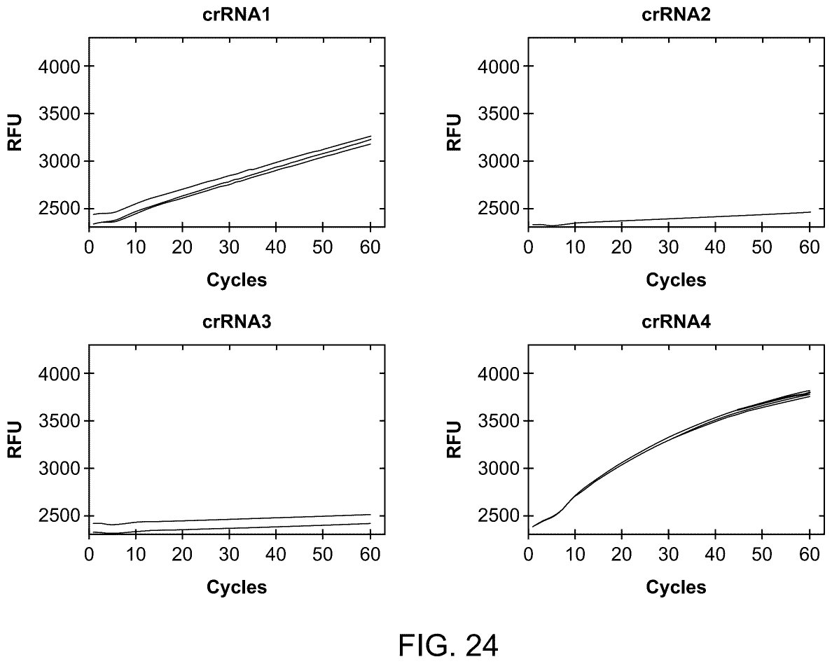

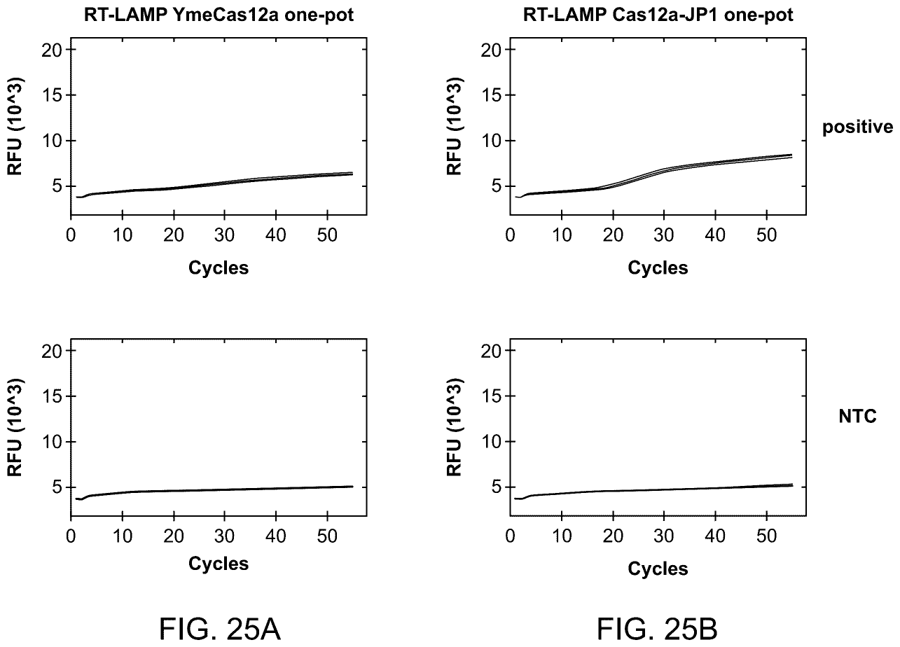

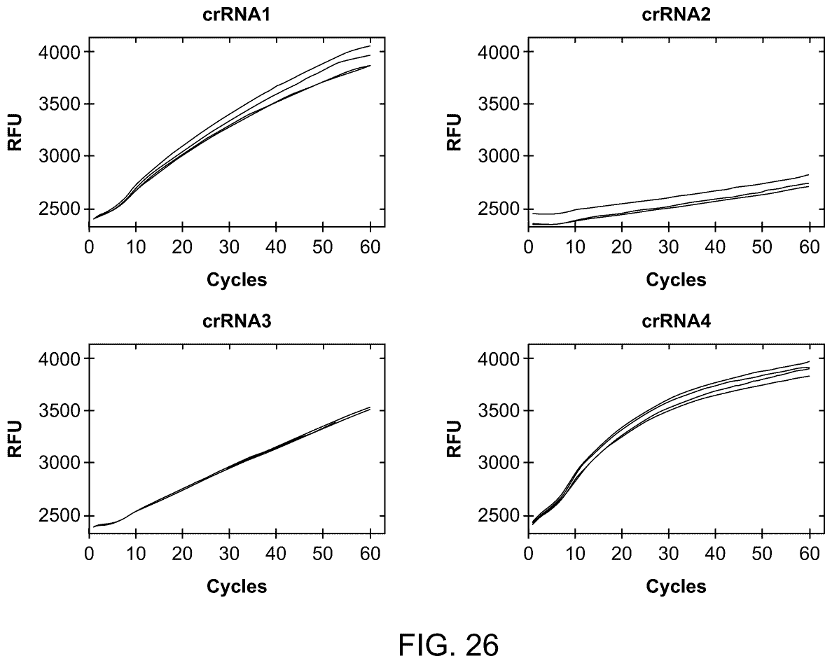

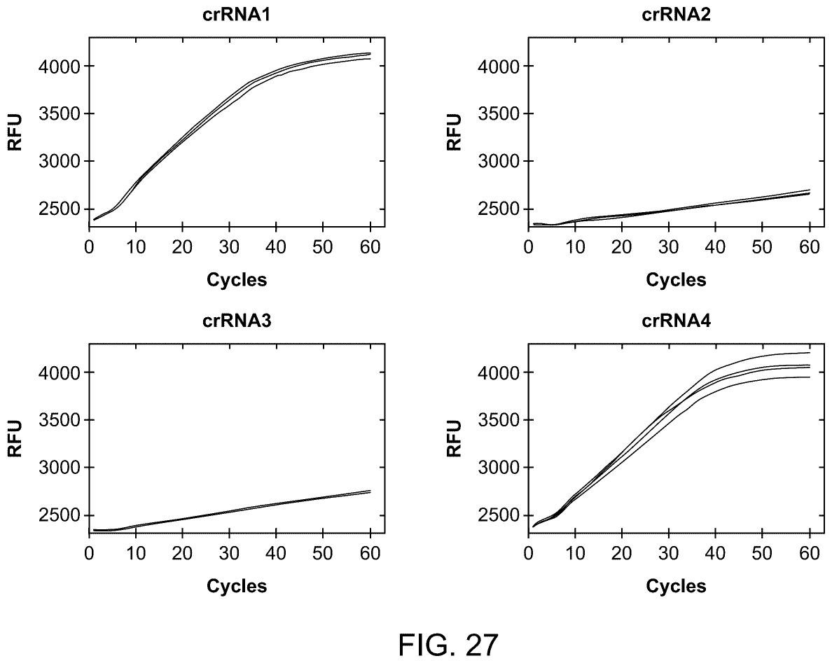

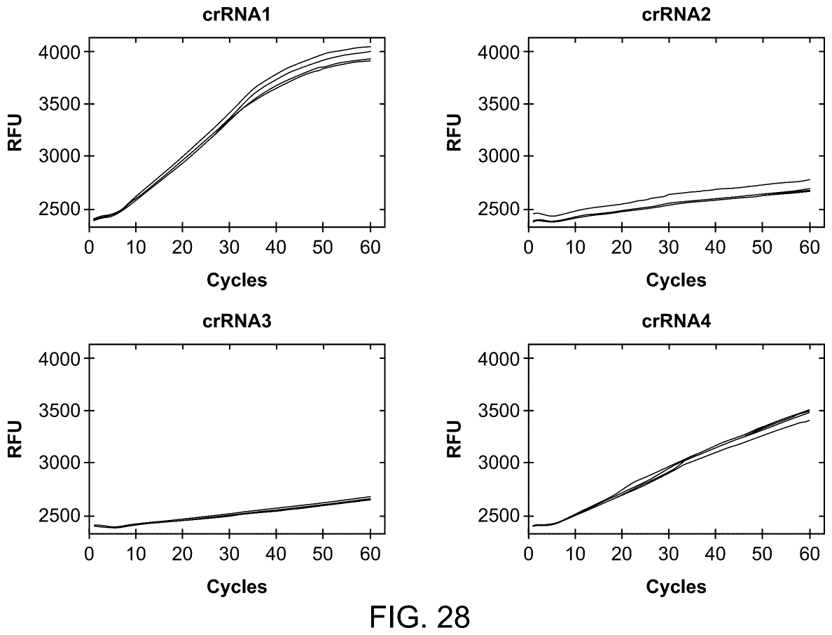

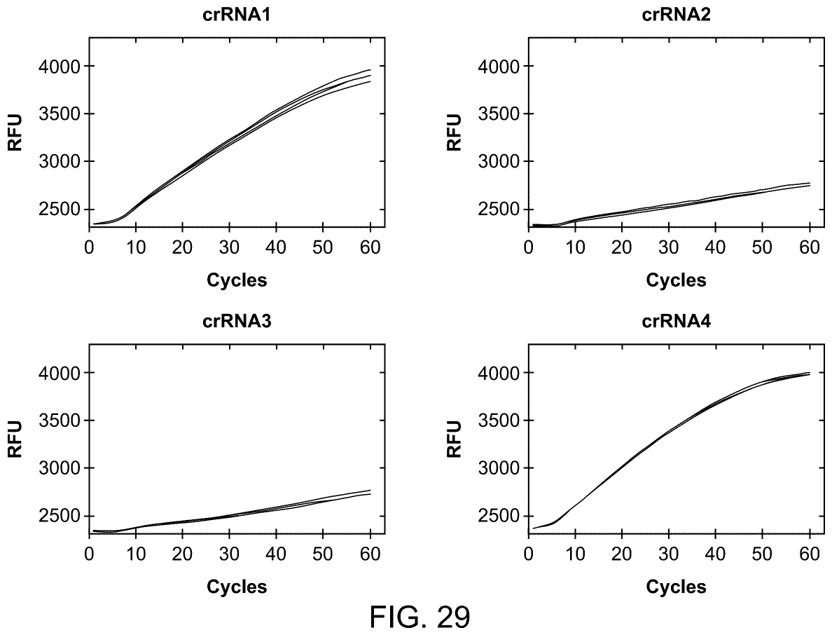

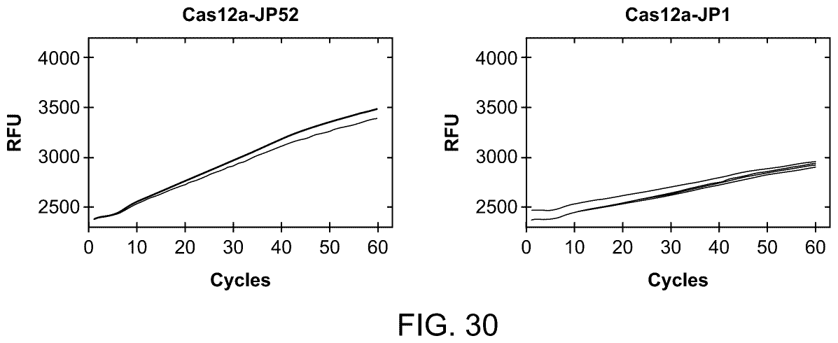

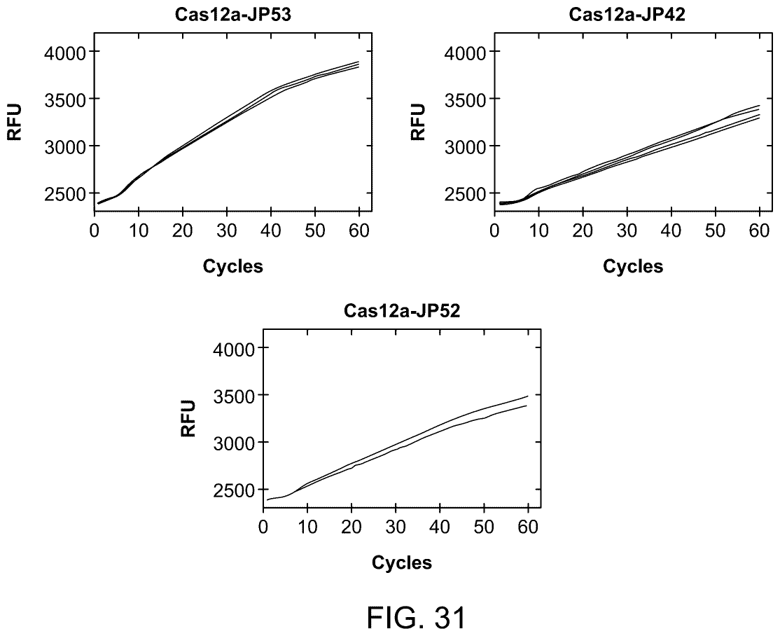

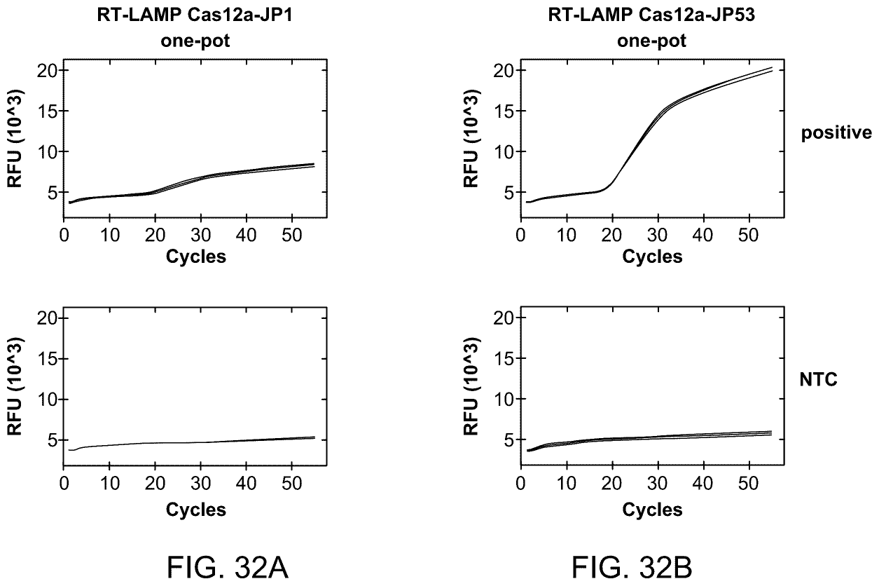

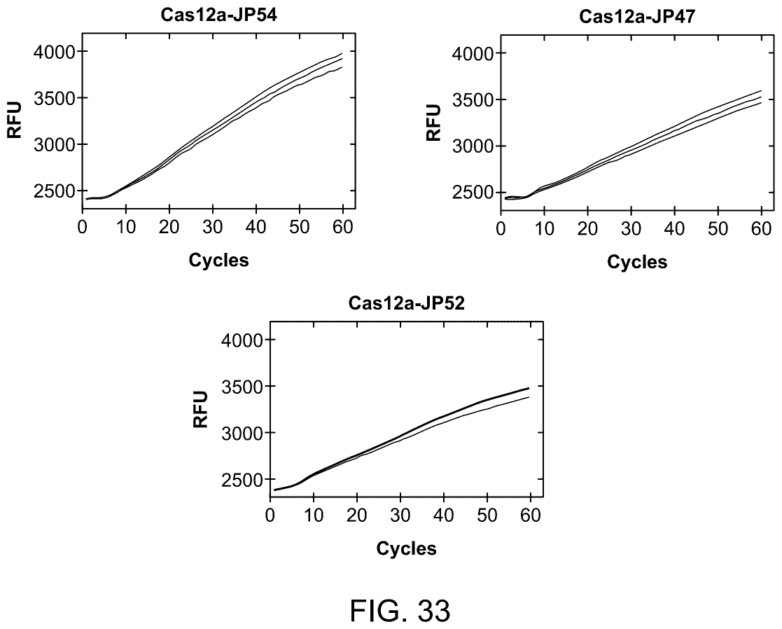

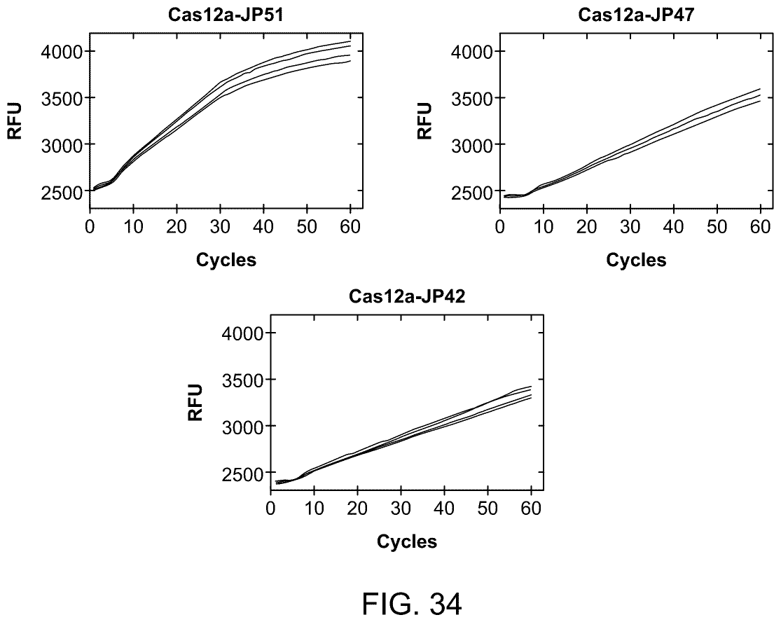

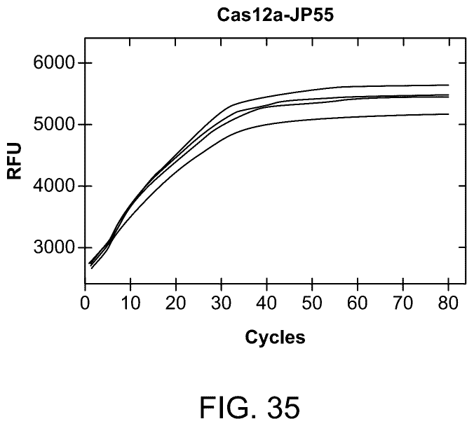

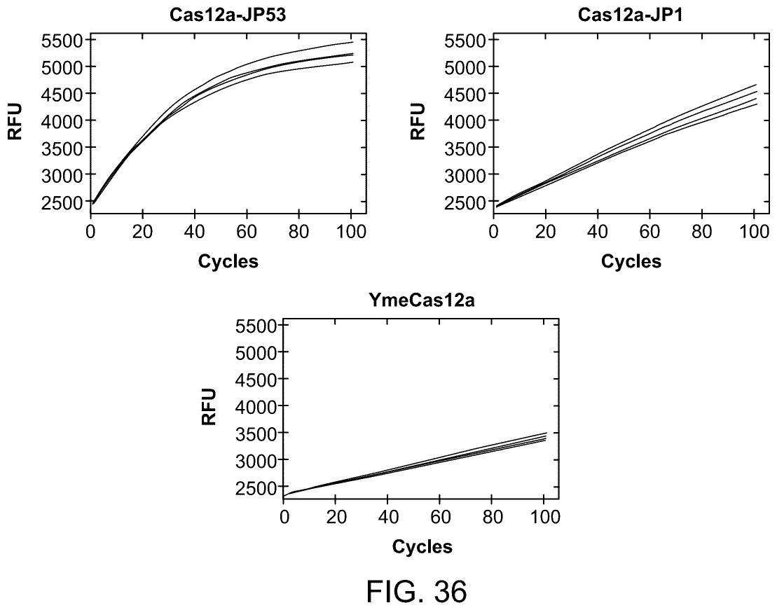

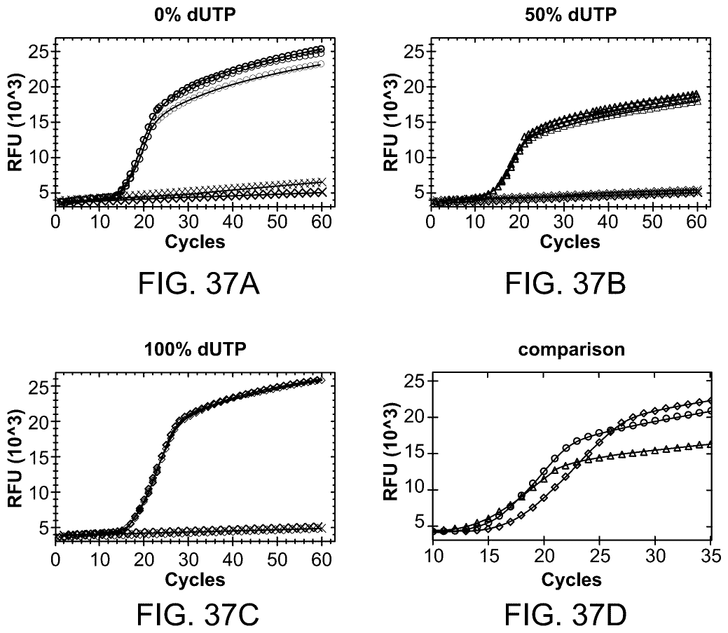

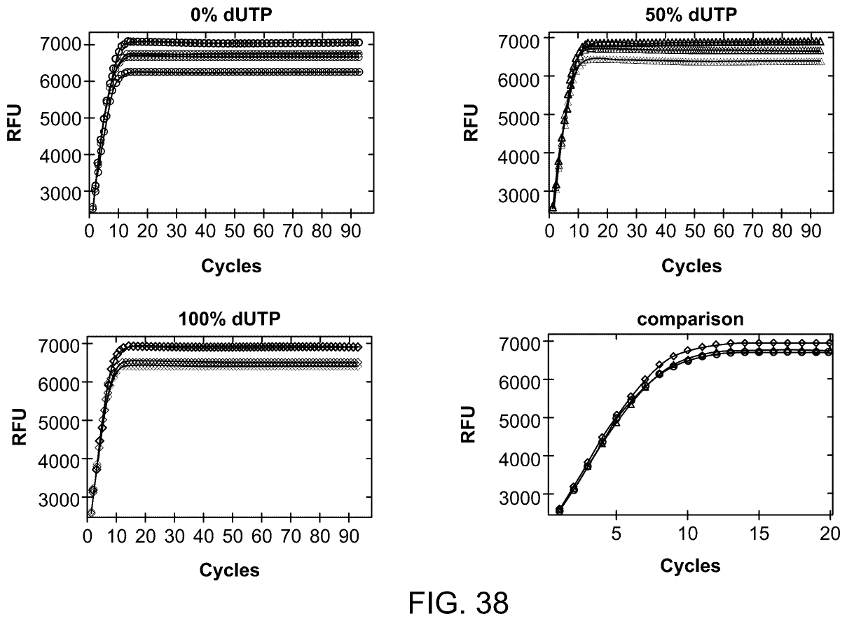

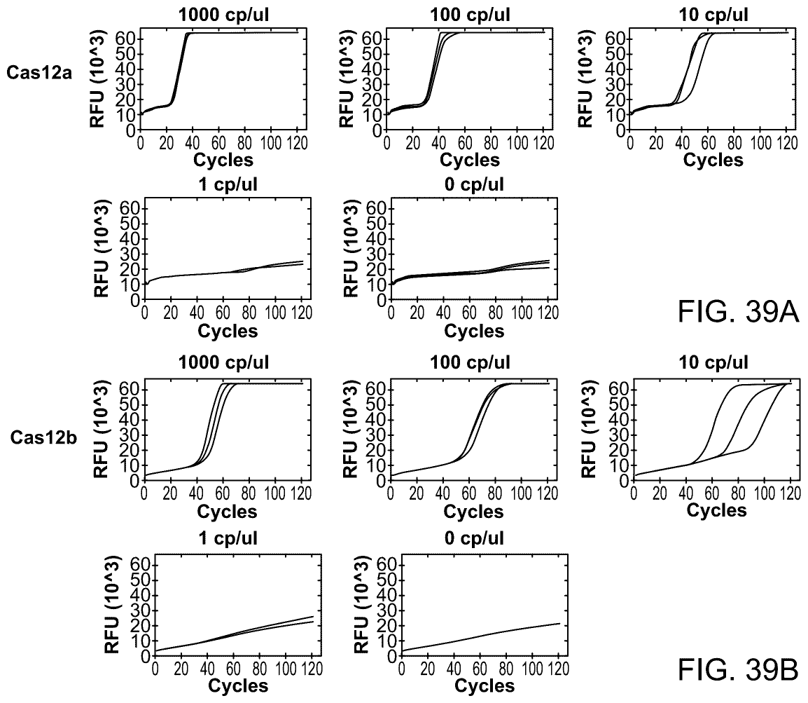

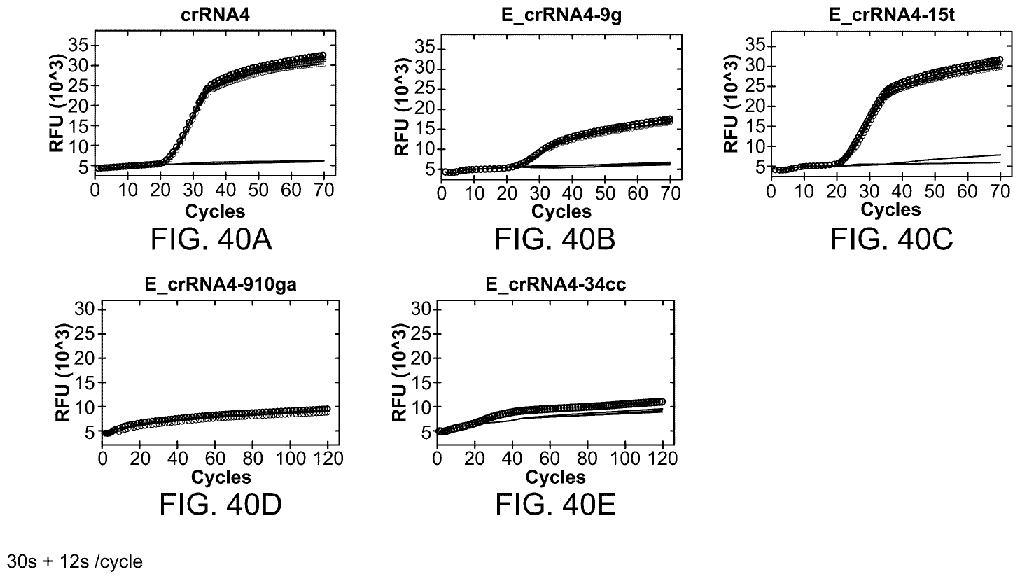

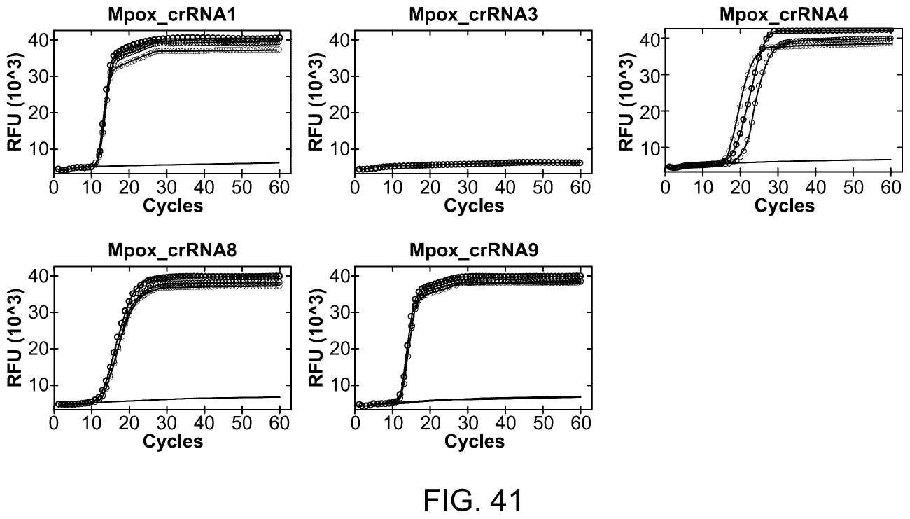

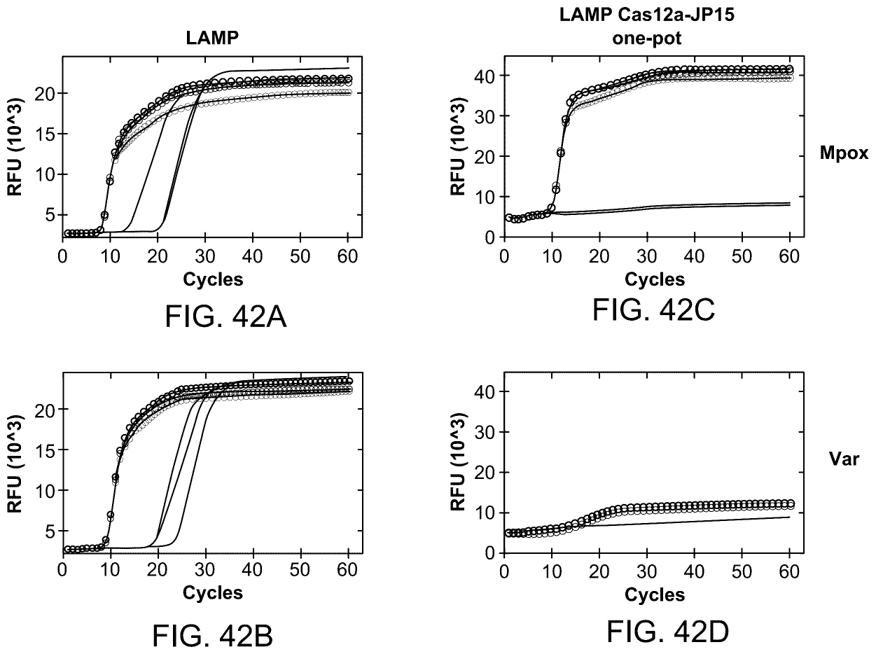

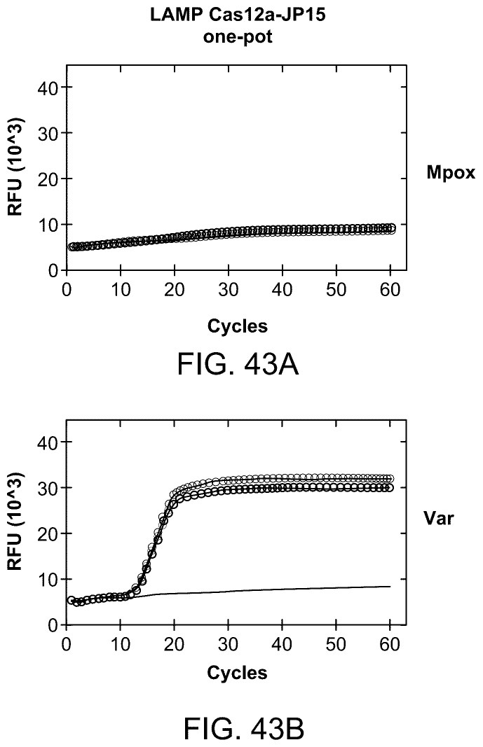

The positive tests with crRNA4 (FIG. 4B) showed prominent FAM fluorescence signal whereas NTC tests showed background signal. This clear presence and absence of signal for testing samples with and without target nucleic acids, respectively, makes the interpretation of one-pot test results simple and straightforward. Further, comparing FIGS. 4A and 4B, successful one-pot Cas12a-coupled RT-LAMP detection was enabled by crRNA4 but not crRNA2, indicating that the likelihood of successful one-pot Cas12a coupled nucleic acid detection is correlated with high crRNA trans nuclease efficiency (compare trans nuclease activity results with crRNA2 and crRNA4 in FIG. 3).

FIG. 5 shows example nucleic acid detection by standalone RT-LAMP (i.e., detection not employing a Cas 12a nuclease). This is to compare with detection coupling RT-LAMP with Cas12a (FIGS. 4A and 4B). Traces show HEX fluorescence that arises from intercalating dye SYTO™ 82 that binds to dsDNA product generated by the RT-LAMP reaction. Top panel shows fluorescence signal from reactions with template (positive, 200 copies/μl synthetic SARS-COV-2 RNA per reaction), and bottom panel shows fluorescence signal from reactions of the no-template control (NTC). Both NTC and positive reactions show fluorescence signal, except that the signal from NTC was delayed (i.e., it required more amplification cycles to appear). Reactions of FIG. 5 were set up from the same master mix for reactions of FIGS. 4A and 4B and monitored on the same 96-well plate, except that equivalent amount of water was substituted for the RNP.

FIG. 6 shows example results of a trans nuclease activity assay using Cas12a-JP15. Each crRNA of E gene (crRNA1-crRNA4) was loaded with Cas12a-JP15 to form an RNP complex, which was activated by cis substrate E gene DNA (E1-PCR). The trans nuclease activity of Cas12a-JP15 RNP-DNA complex was monitored by FAM signal from cleavage of the NZ-GT reporter. Reactions were carried out in 1× modified NEBuffer r2.1 supplemented with 1 mM dNTPs, 50 mM NaCl, and incubated at 55° C. FAM signal was monitored every 15 s in the SYBR channel.

FIGS. 7A-7D show example results of a one-pot reaction coupling Cas12a-JP15 and RT-LAMP reactions. FIG. 7A shows results with crRNA1, FIG. 7B shows results with crRNA2, FIG. 7C shows results with crRNA3, and FIG. 7D shows results with crRNA4. Traces show FAM fluorescence that arises from Cas12a-JP15 trans cleavage of the NZ-GT reporter after activation by the RT-LAMP reaction product. The upper panels of each of FIGS. 7A-7D show reactions with template (positive, 200 copies/μl synthetic SARS-CoV-2 RNA per reaction). The lower panels of each of FIGS. 7A-7D show reactions with no-template control (NTC). The RT-LAMP Cas12a-JP15 one-pot reactions compromise of RT-LAMP and Cas12a reagents in a single reaction mixture, with Cas12a-JP15 and crRNAs targeting E gene of SARS-COV-2. Each cycle was approximately 52 s. As shown, guides crRNA2, crRNA3 and crRNA4 resulted in rapid and specific one-pot detection of the target E gene.

FIG. 8 shows results of nucleic acid detection by standalone RT-LAMP, in comparison to results of FIGS. 7A-7D. Traces show HEX fluorescence that arises from intercalating SYTO™ 82 dye binding with dsDNA generated by RT-LAMP reaction. The upper panel shows reaction with template (positive, 200 copies/μl synthetic SARS-COV-2 RNA per reaction). The lower panel shows reaction with no-template control (NTC). Reactions of FIG. 8 were set up from the same master mix for reactions of FIGS. 7A-7D and monitored on the same 96-well plate, except that equivalent amount of water was substituted for the RNP. Both NTC and positive reactions show fluorescence signals, except that the signal from NTC was delayed, demonstrating that nucleic acid detection by RT-LAMP is not specific due to nonspecific amplifications.

FIG. 9 shows example results of a trans nuclease activity assay using Cas12a-JP19. Each crRNA of E gene (crRNA1-crRNA4) was loaded with Cas 12a-JP19 to form an RNP complex, which was activated by cis substrate E gene DNA (E1-PCR). The trans nuclease activity of Cas12a-JP19 RNP-DNA complex was monitored by FAM signal from cleavage of the NZ-GT reporter. Reactions were carried out in 1× modified NEBuffer r2.1 supplemented with 1 mM dNTPs, 50 mM NaCl, and incubated at 55° C. FAM signal was monitored every 15 s in the SYBR channel.

FIGS. 10A and 10B shows example results of nucleic acid detection by a one-pot reaction coupling Cas 12a-JP19 and RT-LAMP (FIG. 10A) as compared to standalone RT-LAMP (FIG. 10B). Traces in FIG. 10A show FAM fluorescence that arises from Cas12a-JP19 trans cleavage of the NZ-GT reporter after activation by the RT-LAMP reaction product; traces in FIG. 10B show HEX fluorescence that arises from intercalating SYTO™ 82 dye binding with dsDNA generated by RT-LAMP reaction. The upper panels of each of FIGS. 10A and 10B show reactions with template (positive, 200 copies/μl synthetic SARS-COV-2 RNA per reaction). The lower panels of each of FIGS. 10A and 10B show reactions with no-template control (NTC). Reactions were set up from a single master mix and monitored on the same plate under the same conditions. The one-pot reactions comprise RT-LAMP and Cas12a-JP19 reagents, wherein Cas12a-JP19 was loaded with crRNA4 for E gene of SARS-COV-2. The standalone RT-LAMP reactions comprise of RT-LAMP reagents only and used equivalent amount of water to substitute for RNP. Each cycle was approximately 52 s.

FIGS. 11A and 11B show example results of nucleic acid detection by a one-pot reaction coupling Cas 12a-JP16 and SDA (FIG. 11A) as compared to standalone SDA (FIG. 11B). Traces in FIG. 11A show FAM fluorescence that arises from Cas12a-JP16 trans cleavage of the NZ-GT reporter after activation by the SDA reaction product; traces in FIG. 11B show HEX fluorescence that arises from intercalating SYTO™ 82 dye binding with dsDNA generated by SDA reaction. The upper panels of each of FIGS. 11A and 11B show reactions with template (positive, 10 pM E1-PCR DNA). The lower panels of each of FIGS. 11A and 11B show control reactions with no template (NTC). Reactions were set up from a single master mix and monitored on the same plate under the same conditions. The one-pot reactions comprise SDA and Cas12a-JP16 reagents, wherein Cas12a-JP16 was loaded with crRNA4 for E gene of SARS-COV-2. The standalone SDA reactions comprise of SDA reagents only used equivalent amount of water to substitute for RNP. Reactions were carried out at 58° C. Each cycle was approximately 42 s.

In FIG. 11B, both positive and NTC reactions showed fluorescence signal, except that the signal from NTC was delayed, demonstrating that nucleic acid detection by standalone SDA is not specific due to nonspecific amplifications. In contrast, the SDA-Cas12a one-pot reactions (FIG. 11A) showed presence of signal only for positive reaction and not the NTC. Thus, one-pot reaction coupling SDA with Cas12a-JP16 showed improved specificity compared to SDA.

FIG. 12 shows analysis of results by agarose gel from the same reactions as shown in FIG. 11B. Products of SDA reactions (3 repeats) were loaded onto 4% agarose gel, stained with Gel green, and visualized under UV. The expected product band was labeled with an asterisk. Other bands on the gel were non-specific amplification products.

This conventional method to analyze SDA products (i.e., by gel electrophoresis) is time consuming and runs the risk of carry-over contamination of future tests due to possible aerosolization of SDA products when the reaction vessel was opened to extract reaction products for gel electrophoresis. Detection of nucleic acid by one-pot test coupling SDA with Cas12a, as shown in FIGS. 11A, requires no further treatment of reaction products once reactions were set up, thus is highly simple and rapid, and prevents carry-over contamination.

FIGS. 13A and 13B show example results of nucleic acid detection by a one-pot reaction coupling Cas 12a-JP15 and SDA (FIG. 13A) as compared to standalone SDA (FIG. 13B). Traces in FIG. 13A show FAM fluorescence that arises from Cas12a-JP15 trans cleavage of the NZ-GT reporter after activation by the SDA reaction product; traces in FIG. 13B show HEX fluorescence that arises from intercalating SYTO™ 82 dye binding with dsDNA generated by SDA reaction. The upper panels of each of FIGS. 13A and 13B show reactions with template (positive, 10 pM E1-PCR). The lower panels of each of FIGS. 13A and 13B show reactions with no-template control (NTC). Reactions were set up from a single master mix and monitored on the same plate under the same conditions. The one-pot reactions in FIG. 13A comprise SDA and Cas12a-JP15 reagents, where Cas12a-JP15 was loaded with crRNA4 for E gene of SARS-COV-2. The standalone SDA reactions in FIG. 13B used equivalent amount of water to substitute for RNP. Reactions were carried out at 58° C. Each cycle was approximately 42 s.

FIG. 14 shows example results of a trans nuclease activity assay using Cas12a-JP13 at 55° C. Cas12a-JP13 was loaded with crRNA2 or crRNA4 of E gene of SARS-COV-2 to form an RNP complex, which was activated by cis substrate E gene DNA (E1-PCR). The trans nuclease activity of Cas12a-JP13 was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

FIGS. 15A and 15B show example results of nucleic acid detection by a one-pot reaction coupling Cas 12a-JP13 and SDA (FIG. 15A) as compared to standalone SDA (FIG. 15B). Traces in FIG. 15A show FAM fluorescence that arises from Cas12a-JP13 trans cleavage of the NZ-GT reporter after activation by the SDA reaction product; traces in FIG. 15B show HEX fluorescence that arises from intercalating SYTO™ 82 dye binding with dsDNA generated by SDA reaction. The upper panels of each of FIGS. 15A and 15B show reactions with template (positive, 10 pM E1 PCR product). The lower panels of each of FIGS. 15A and 15B show reactions without template (NTC). Reactions were set up from a single master mix and monitored on the same plate under the same conditions. The one-pot reactions in FIG. 15A comprise SDA and Cas12a-JP13 reagents, where Cas12a-JP13 was loaded with crRNA4 for E gene of SARS-COV-2. The standalone SDA reactions in FIG. 15B used equivalent amount of water to substitute for RNP. Reactions were carried out at 58° C. Each cycle was approximately 42 s.

FIG. 16 shows example results of a trans nuclease activity assay of Cas12a-JP13 at 37° C. Cas12a-JP13 was loaded with crRNA2 or crRNA4 of E gene of SARS-COV-2 to form an RNP complex, which was activated by cis substrate E gene DNA (E1-PCR). The trans nuclease activity of Cas12a-JP13 was monitored by FAM signal from cleavage of NZ-GT reporter. Reactions were carried out in 1× modified NEBuffer r2.1, supplemented with 50 mM NaCl, 1 mM dNTPs and incubated at 37° C. FAM signal was monitored every 15 s.

FIGS. 17A and 17B show example results of nucleic acid detection by a one-pot reaction coupling Cas12a-JP13 and RPA (FIG. 17A) as compared to standalone RPA (FIG. 17B). Traces in FIG. 17A show FAM fluorescence that arises from Cas12a-JP13 trans cleavage of the T15 reporter after activation by the RPA reaction product. Traces in FIG. 17B show background FAM fluorescence from uncleaved T15 reporter. The upper panels of each of FIGS. 17A and 17B show reactions with template (positive, 4 pM E1-PCR). The lower panels of each of FIGS. 17A and 17B show reactions without template (NTC). Reactions were set up from a single master mix and monitored on the same plate under the same conditions. The one-pot reactions in FIG. 17A comprise RPA and Cas12a-JP13 reagents, where Cas 12a-JP13 was loaded with crRNA4 for E gene of SARS-CoV-2. Standalone RPA reactions (FIG. 17B) used equivalent amount of water to substitute for RNP. Reactions were carried out at 42° C. Each cycle was approximately 37 s.

The one-pot tests coupling RPA with Cas12a (FIG. 17A) showed a prominent FAM fluorescence signal in the presence of target and background signal for the NTC. This clear presence and absence of signal for samples with and without target nucleic acids, respectively, makes the interpretation of one-pot nucleic acid detection results simple and straightforward. Traces in FIG. 17B show background FAM fluorescence from uncleaved T15 reporter. Due to the non-specific amplifications cooccurring with target amplification, detection of standalone RPA product requires more involving methods, such as by gel electrophoresis (shown in FIG. 18).

FIG. 18 shows example results of an RPA product analysis by agarose gel. Reactions were the same as shown in FIG. 17B. Four microliter reaction products from one repeat were mixed with DNA loading dye and loaded onto 3% (w/v) agarose gel, stained with Gel green, and visualized under UV. The expected product band was labeled with an asterisk. Conventional analysis of RPA products (for instance, by gel electrophoresis) is time consuming and runs the risk of cross-contamination due to aerosolization of RPA products during the multiple operational steps that require opening of the reaction vessel.

FIGS. 19A and 19B show example results of nucleic acid detection by a one-pot reaction coupling Cas 12a-JP15 and RPA (FIG. 19A) as compared to standalone RPA (FIG. 19B). Traces in FIG. 19A show FAM fluorescence that arises from Cas12a-JP15 trans cleavage of the T15 reporter after activation by the RPA reaction product. Traces in FIG. 19B show background FAM fluorescence from uncleaved T15 reporter. The upper panels of each of FIGS. 19A and 19B show reactions with template (positive, 4 pM E1-PCR). The lower panels of each of FIGS. 19A and 19B show reactions without template (NTC). Reactions were set up from a single master mix and monitored on the same plate under the same conditions. The one-pot reactions in FIG. 19A comprise RPA and Cas12a-JP15 reagents, where Cas12a-JP15 was loaded with crRNA4 for E gene of SARS-CoV-2. Standalone RPA reactions (FIG. 19B) used equivalent amount of water to substitute for RNP. Reactions were carried out at 42° C. Each cycle was approximately 37 s.

FIG. 20 shows results example of a trans nuclease activity assay of Cas12a-JP31 at 37° C. Cas12a-JP31 was loaded with crRNA2 or crRNA4 of E gene of SARS-COV-2 to form an RNP complex, which was activated by cis substrate E gene DNA (E1-PCR). The trans nuclease activity of Cas12a-JP31 was monitored by FAM signal from cleavage of the NZ-GT reporter. Reactions were carried out in 1× modified NEBuffer r2.1 supplemented with 50 mM NaCl, 1 mM dNTPs and incubated at 37° C. FAM signal was monitored every 15 s in the SYBR channel.

FIG. 21 shows example results of a trans nuclease activity assay using Cas12a-JP31 at 55° C. Cas12a-JP31 was loaded with crRNA2 or crRNA4 of E gene of SARS-COV-2 to form an RNP complex, which was activated by cis substrate E gene DNA (E1-PCR). The trans nuclease activity of Cas12a-JP31 was monitored by FAM signal from cleavage of the NZ-GT reporter. Reactions were carried out in 1× modified NEBuffer r2.1 supplemented with 50 mM NaCl, 1 mM dNTPs and incubated at 55° C. FAM signal was monitored every 15 s in the SYBR channel.

FIG. 22 shows example results of a trans nuclease activity assay using Cas12a-JP29. Cas 12a-JP29 was loaded with crRNA2 or crRNA4 of E gene of SARS-COV-2 to form an RNP complex, which was activated by cis substrate E gene DNA (E1-PCR). The trans nuclease activity of Cas 12a-JP29 was monitored by FAM signal from cleavage of the NZ-GT reporter. Reactions were carried out in 1× modified NEBuffer r2.1 supplemented with 50 mM NaCl, 1 mM dNTPs and incubated at 55° C. FAM signal was monitored every 15 s in the SYBR channel.

FIG. 23 shows example results of a trans nuclease activity assay using a wildtype Cas 12a from Yellowstone metagenome (YmeCas12a). The YmeCas12a was loaded with one of the four crRNAs for E gene (crRNA1-crRNA4) of SARS-COV-2 to form an RNP complex, which was activated by cis substrate E gene DNA (E1-PCR). The trans nuclease activity of YmeCas12a was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

FIG. 24 shows example results of a trans nuclease activity assay using Cas12a-JP1, an engineered variant of YmeCas12a. The Cas12a-JP1 was generated by swapping the YmeCas12a amino acids 98-117 (LKLKSEIQKLKGEKKQKEAN; SEQ ID NO:17) with amino acids 82-88 (RKKTRTE; SEQ ID NO:74) of Cas 12a from Lachnospiraceae bacterium (LbaCas12a). Cas12a-JP1 was loaded with one of the four crRNAs for E gene (crRNA1-crRNA4) of SARS-COV-2 to form an RNP complex, which was activated by cis substrate E gene DNA (E1-PCR). The trans nuclease activity of Cas12a-JP1 was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was recorded every 15 s in the SYBR channel. Variant Cas12a-JP1 exhibited higher trans nuclease activity than YmeCas12a (FIG. 23) with all four guides under the conditions tested, as estimated by the rate of RFU increase in the initial linear phase.

FIGS. 25A and 25B show example results of nucleic acid detection by a one-pot reaction coupling RT-LAMP with wildtype YmeCas12a (FIG. 25A) or variant Cas12a-JP1 (FIG. 25B). Traces show FAM fluorescence that arises from Cas12a trans cleavage of the NZ-GT reporter. The top panel shows reactions with template (positive, 200 copies/μl synthetic SARS-COV-2 RNA per reaction). The bottom panel shows reactions with no-template control (NTC). All reactions were set up from a single master mix and differentiated by addition of the specific RNP and target. Both tests used crRNA4 for E gene of SARS-CoV-2. Reactions were incubated at 55° C. Each cycle was approximately 52 s.

The one-pot test with Cas12a-JP1 showed a more obvious FAM fluorescence signal in comparison to YmeCas12a when positive sample was present (compare top panels of FIGS. 25A and 25B). These results demonstrated that Cas12a-JP1 outperforms YmeCas12a in one-pot nucleic acid assay coupling Cas 12a with RT-LAMP.

FIG. 26 shows example results of a trans nuclease activity assay using variant Cas12a-JP42. The Cas12a-JP42 was generated by making G1019K mutation to Cas12a-JP1. Cas12a-JP42 was loaded with one of the four crRNAs for E gene (crRNA1-crRNA4) of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

Variant Cas12a-JP42 exhibited higher trans nuclease activity than Cas12a-JP1 (FIG. 24) with all four guides under the conditions tested, as estimated by the rate of RFU increase in the initial linear phase.

FIG. 27 shows example results of a trans nuclease activity assay using variant Cas12a-JP45. The Cas12a-JP45 was generated by fusing Cas12a-JP1 with the DNA-binding protein Sso7d at the C-terminal end. In the assay, Cas12a-JP45 was loaded with one of the four crRNAs for E gene (crRNA1-crRNA4) of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

Variant Cas12a-JP45 exhibited higher trans nuclease activity than Cas12a-JP1 (FIG. 24) with all four guides under the conditions tested, as estimated by the rate of RFU increase in the initial linear phase.

FIG. 28 shows example results of a trans nuclease activity assay using the variant Cas12a-JP47. Variant Cas12a-JP47 was generated by fusing Cas12a-JP1 with the DNA-binding protein ET-SSB at the N-terminal end. Cas12a-JP47 was loaded with one of the four crRNAs for E gene (crRNA1-crRNA4) of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

Variant Cas12a-JP47 exhibited higher trans nuclease activity than Cas12a-JP1 (FIG. 24) with all four guides except crRNA4under the conditions tested, as estimated by the rate of RFU increase in the initial linear phase.

FIG. 29 shows example results of a trans nuclease activity assay using the variant Cas12a-JP48. Variant Cas12a-JP48 was generated by fusing Cas12a-JP1 with the DNA-binding protein ET-SSB at the C-terminal end. Cas12a-JP48 was loaded with one of the four crRNAs for E gene (crRNA1-crRNA4) of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s in the SYBR channel.

Variant Cas12a-JP48 exhibited higher trans nuclease activity than Cas12a-JP1 (FIG. 24) with all four guides except crRNA4under the conditions tested, as estimated by the rate of RFU increase in the initial linear phase.

FIG. 30 shows example results of a trans nuclease activity assay using variant Cas12a-JP52 (left) in comparison to Cas12a-JP1 (right). The Cas12a-JP52 was generated by the 11155R substitution in Cas12a-JP1. Reactions were performed side by side for direct comparison. Both Cas12a variants were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 100 mM NaCl, 1 mM dNTPs and incubated at 55° C. FAM signal was monitored every 15 s in the SYBR channel. Variant Cas12a-JP52 exhibited higher trans nuclease activity than Cas12a-JP1 under the conditions tested, as estimated by the rate of RFU increase in the initial linear phase.

FIG. 31 shows example results of a trans nuclease activity assay using the variant Cas12a-JP53 (upper left) in comparison to Cas12a-JP42 (upper right) and Cas12a-JP52 (bottom). The Cas12a-JP53 was generated by combining the two amino acid substitutions derived from Cas12a-JP52 (11155R) and Cas12a-JP42 (G1019K) of Cas12a-JP1. Reactions were performed side by side for direct comparison. The Cas12a variants were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 100 mM NaCl, 1 mM dNTPs and incubated at 55° C. FAM signal was monitored every 15 s in the SYBR channel. Variant Cas12a-JP53 exhibited higher trans nuclease activity than Cas12a-JP42 or Cas12a-JP52 under the conditions tested, as estimated by the rate of RFU increase in the initial linear phase.

FIGS. 32A and 32B show example results of nucleic acid detection by a one-pot reaction coupling RT-LAMP with Cas12a-JP1 (FIG. 32A) or Cas12a-JP53 (FIG. 32B). Traces show FAM fluorescence that arises from Cas12a trans cleavage of the NZ-GT reporter. The top panel shows reactions with template (positive, 200 copies/μl synthetic SARS-COV-2 RNA per reaction) and the bottom panel shows reactions with NTC. All reactions were set up from a single master mix and differentiated by the specific RNP and template. The Cas 12a variants were loaded with crRNA4 for E gene of SARS-COV-2. Reactions were incubated at 55° C. Each cycle was approximately 52 s.

The one-pot test with Cas12a-JP53 showed a much higher and prominent FAM fluorescence signal in comparison to Cas 12a-JP1 in the presence of template (compare top panels of FIGS. 32A and 32B). These results demonstrated that Cas12a-JP53 showed improved performance than Cas12a-JP1 in one-pot nucleic acid detection coupling Cas12a with RT-LAMP.

FIG. 33 shows example results of a trans nuclease activity assay using the variant Cas12a-JP54 (upper left) in comparison to Cas12a-JP47 (upper right) and Cas12a-JP52 (bottom). The Cas12a-JP54 was generated from Cas12a-JP1 by combining the amino acid substitution 11155R (as in Cas12a-JP52) and N-terminal end fusion of the DNA-binding protein ET-SSB (as in Cas12a-JP47). Reactions were performed side by side for direct comparison. The Cas 12a variants were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 100 mM NaCl, 1 mM dNTPs and incubated at 55° C. FAM signal was monitored every 15 s in the SYBR channel. Variant Cas12a-JP54 exhibited higher trans nuclease activity than Cas12a-JP47 or Cas12a-JP52 under the conditions tested, as estimated by the rate of RFU increase in the initial linear phase.

FIG. 34 shows example results of a trans nuclease activity assay using the variant Cas12a-JP51 (upper left) in comparison to Cas12a-JP47 (upper right) and Cas12a-JP42 (bottom). The Cas12a-JP51 was generated by combining the amino acid substitution G1019K (as in Cas12a-JP42) and the N-terminal end fusion with the DNA-binding protein ET-SSB (as in Cas12a-JP47). Reactions were performed side by side for direct comparison. The Cas12a variants were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 100 mM NaCl, 1 mM dNTPs and incubated at 55° C. FAM signal was monitored every 15 s in the SYBR channel. Variant Cas12a-JP51 exhibited higher trans nuclease activity than Cas12a-JP47 or Cas 12a-JP42under the conditions tested, as estimated by the rate of RFU increase in the initial linear phase.

FIG. 35 shows example results of a trans nuclease activity assay using the variant Cas12a-JP55. The Cas12a-JP55 was generated from Cas12a-JP51 by additionally introducing the amino acid substitution 11155R (as in Cas12a-JP52). The Cas12a variants were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl, 1 mM dNTPs and incubated at 55° C. FAM signal was monitored every 15 s.

FIG. 36 shows example results of a trans nuclease activity assay using the variant Cas12a-JP53 (upper left) in comparison to Cas12a-JP1 (upper right) and wildtype YmeCas12a (bottom) at 37° C. Reactions were performed side by side for direct comparison. The Cas 12a enzymes were loaded with crRNA4 for E gene of SARS-COV-2 to form an RNP complex, which was activated by cis substrate E gene DNA (E1-PCR). The trans nuclease activity was monitored by FAM signal from cleavage of the NZ-GT reporter. Reactions were carried out in 1× modified NEBuffer r2.1, supplemented with 50 mM NaCl and incubated at 37° C. FAM signal was monitored every 10 s in the SYBR channel. Variant Cas12a-JP53 exhibited higher trans nuclease activity than Cas12a-JP1 or YmeCas12a with crRNA4 at 37° C. under the conditions tested, as estimated by the rate of RFU increase in the initial linear phase.

FIGS. 37A-37D shows example results of nucleic acid detection by a one-pot reaction coupling RT-LAMP with Cas12a-JP15 with different amounts of dUTP in the reaction. Traces show FAM fluorescence that arises from Cas 12a trans cleavage of the NZ-GT reporter. The reactions with 0% dUTP (FIG. 37A) were supplied with 1 mM of each dNTPs, i.e., dATP, dCTP, dGTP, and dTTP, and no dUTP. The reactions with 50% dUTP (FIG. 37B) were the same as in condition A except for the addition of 0.5 mM dUTP, i.e., 0.5 mM dUTP in addition to 1 mM of each of the dATP, dCTP, dGTP, and dTTP. The reactions with 100% dUTP (FIG. 37C) substituted dTTP with dUTP of the reactions with 0% dUTP, i.e., 1 mM of dATP, dCTP, dGTP, and dUTP each. The reactions with template (400 copies/μl synthetic SARS-COV-2 RNA per reaction) were depicted with circles (FIG. 37A), triangles (FIG. 37B), or diamonds (FIG. 37C). The reactions with NTC were shown with crosses. Comparison of the three different conditions was shown in FIG. 37D, with one representative trace from each condition for clarity. The Cas12a-JP15 was loaded with crRNA4 for E gene of SARS-COV-2 in the one-pot reactions. Reactions were incubated at 55° C. Each cycle is approximately 72 s.

The time of detection of E gene, as shown by FIG. 37D was indistinguishable for 0% and 50% dUTP, and slightly delayed for 100% dUTP. The condition of 50% dUTP, i.e. dNTPs with the addition of 0.5× molar ratio of dUTP, was usually applied in carry-over prevention procedures in LAMP in combination with uracil-DNA glycosylase (UDG). Thus, these results indicate that the one-pot nucleic acid detection method coupling LAMP with Cas12a would be compatible with carry-over prevention practice.

FIG. 38 shows example results of a trans nuclease activity assay using the variant Cas12a-JP15 activated by cis substrate DNA that was generated from dNTPs incorporated with 0% dUTP, 50% dUTP, or 100% dUTP. The definition of the dUTP conditions is the same as in FIGS. 37A-37D. Comparison of the trans nuclease activity used one representative trace from each condition during the 0-20 cycles for clarity.

To minimize the errors in quantification of the cis substrates after purification, the trans nuclease activity assay was performed with limiting RNP and excess cis substrate. This way the same amount of Cas 12a-crRNA-cis DNA product complex would react with excess reporter to test the effect of dU-incorporation in DNA on trans nuclease activity. Specifically, the Cas12a-JP15 enzymes (15 nM) was loaded with 1.5× crRNA4 for E gene of SARS-COV-2 to form an RNP complex, which was activated by 20 nM cis substrate E gene DNA (E1-PCR) generated from dNTPs with the three different dUTP profiles described in FIGS. 37-37D. The trans nuclease activity was monitored by FAM signal from cleavage of the NZ-GT reporter, which was included to a final concentration of 300 nM. Reactions were carried out in 1× NEBuffer r2.1, supplemented with 1 mM dNTPs, 1 mM TCEP, and incubated at 55° C. Fluorescence signals were recorded every 15 s in all channels.

The presence of dU in the cis substrate DNA does not affect the Cas12a-JP15 trans nuclease efficiency under the conditions tested, as estimated by the rate of RFU increase in the initial linear phase.

FIGS. 39A and 39B show example results comparing nucleic acid detection by one-pot reaction coupling RT-LAMP with Cas12a-JP15 (FIG. 39A) or AapCas12b (FIG. 39B). Traces show FAM fluorescence from Cas12a-JP15 or Alicyclobacillus acidiphilus Cas 12b (AapCas12b) trans cleavage of the NZ-GT reporter or T5 reporter, respectively. Conditions for detection with AapCas12b in one-pot followed protocol reported by Joung J, et al. N Engl J Med. 2020 Oct. 8; 383 (15): 1492-1494.doi: 10.1056/NEJMc2026172. Minor modifications (i.e. 2 uM T5 reporter instead of the reported 0.2 uM reporter) were used to get the best signal. Condition for detection with Cas12a-JP15 were following protocol described in Example 2, except that it included 1.2 mM dNTPs, 4 uM of the NZ-GT reporter, 200 nM RNP, and incubated at 60° C. Guide RNA Cas12b guide was used for AapCas12b and N2_RNAI was used for Cas12a-JP15 for the one-pot reactions. Synthetic SARS-COV-2 RNA was added at 0-1,000 copies/μl per reaction as indicated on top of the figures. Reactions were set up side by side and monitored on the same plate for comparison. Reactions were incubated at 60° C. Each cycle was approximately 42 s.

Both Cas12a-JP15 and AapCas12b enabled specific detection of gene N2 as low as 10 copies/μl per reaction, but the reactions with Cas12a-JP15 showed earlier signal detection (i.e., faster readout) and higher signal-to-noise ratio (i.e., lower background) than those with AapCas12b.

FIG. 40A-40E show example results of nucleic acid detection by a one-pot reaction coupling RT-LAMP with Cas12a-JP15 with one or two mismatches in the crRNA spacer to the target. Traces show FAM fluorescence from Cas12a-JP15 trans cleavage of the 10C reporter. Cas12a-JP15 was loaded with guide RNA whose spacer perfectly matches the targeting E gene of SARS-COV-2 (FIG. 40A, crRNA4), or with C to G change at position 9 (FIG. 40B, E_crRNA4-9g), or with A to U change at position 15 (FIG. 40C, E_crRNA4-15t), or with C to G change at position 9 and U to A change at position 10 (FIG. 40D, E_crRNA4-910ga), or with G to C change at position 3 and G to C change at position 4 (FIG. 40E, E_crRNA4-34cc). In each figure, reactions with template (200 copies/μl synthetic SARS-COV-2 RNA per reaction) were depicted with circles and the corresponding NTC were shown as gray lines. The one-pot reactions compromise of RT-LAMP and Cas12a reagents in a single reaction mixture. Reactions were incubated at 55° C. Each cycle was approximately 42 s.

The crRNAs with single and double mismatches mimic the conditions when the same crRNA was used but target nucleic acids harbor single nucleotide polymorphisms (SNPs). Reactions with crRNA that have perfectly matched spacer to the target, shown in FIG. 40A, showed prominent FAM signal, indicating the presence of the targeting nucleic acids. Reactions with crRNA that has a single mismatch in the spacer, shown in FIGS. 40B and 40C, showed reduced signal level (final RFU level, FIG. 40B) or no change from perfect match guide (FIG. 40C), indicating that single SNP may affect one-pot nucleic acid detection depending on its position. Reactions with crRNA that has consecutive double mismatches at the 9^{th} and 10^{th} positions reduced the Cas 12a signal to background level (FIG. 40D), whereas consecutive double mismatches at the 3^{rd} and 4^{th} positions showed a signal slightly higher than NTC (FIG. 40E), indicating that two consecutive mismatches, especially at 9^{th} and 10^{th} positions, significantly compromise Cas12a-JP15 activity in one-pot. These results provide guidelines in crRNA design to enable SNP detection in one-pot nucleic acid detections.

FIG. 41 shows example results of nucleic acid detection by a one-pot reaction coupling Cas12a-JP15 with LAMP to detect dsDNA virus Mpox (previously known as Monkeypox). Five guide RNAs (SEQ ID NO:26-30) were designed on the expected NIR gene LAMP amplicon, and each was loaded to Cas12a-JP15 to form an RNP complex in the one-pot assay. Traces show FAM fluorescence from Cas12a-JP15 trans cleavage of the 10C reporter. In each figure, reactions with template (2000 copies/μl synthetic monkey Mpox NIR gBlock gene fragment per reaction) were depicted with circles and the corresponding NTC were shown as gray lines. The LAMP Cas 12a-JP15 one-pot reactions compromise of LAMP and Cas 12a reagents in a single reaction mixture. Reactions were incubated at 55° C. Each cycle was approximately 60 s.

All five guides except for Mpox_crRNA3 resulted in rapid, efficient, and specific one-pot detection of the Mpox NIR gene.

FIGS. 42A-42D show example results of NIR gene detection by standalone LAMP (FIG. 42A and FIG. 42B) as compared to one-pot reaction coupling LAMP with Cas12a-JP15 (FIGS. 42C and 42D). Traces in FIG. 42A and FIG. 42B show HEX fluorescence from intercalating dye SYTO™ 82 binding to DNA from amplification reaction. Traces in FIG. 42C and FIG. 42D show FAM fluorescence from Cas12a-JP15 trans cleavage of the 10C reporter. Synthetic gBlock of Mpox NIR (Mpox, 42A and 42C) and variola virus NIR mimic (Var, 42B and 42D) as described in Li et al., were used as template at 2000 copies/μl in the reactions. Reactions with template were depicted with circles, and the corresponding NTC were shown as gray lines. The LAMP Cas12a-JP15 one-pot reactions compromise LAMP and Cas12a reagents in a single reaction mixture, whereas the standalone LAMP reactions comprise of LAMP reagents only. Reactions were incubated at 55° C. Each cycle was approximately 60 s.

With standalone LAMP, both Mpox and Var DNA may be detected, with slightly delayed signal in detection of Var (compare FIG. 42B with FIG. 42A) due to single nucleotide polymorphisms (SNPs) on the primers. Thus, it is not easy to differentiate Mpox and Var by LAMP. With one-pot reaction coupling LAMP with Cas12a-JP15, guide Mpox_crRNA1 was used. The spacer of Mpox_crRNA1 (UGUGCAAUAAUUGGACUUUG; e.g., nucleotides 21-40 of SEQ ID NO:26) matches the target region of Mpox (TGTGCAATAATTGGACTTTG; e.g., nucleotides 310-329 of SEQ ID NO: 47) perfectly but has a single mismatch on the 9^{th} position compared to target region of Var (TGTGCAATCATTGGACTTTG; e.g., nucleotides 310-329 of SEQ ID NO:48). The one-pot assay with Mpox showed prominent fluorescence signal and almost background level fluorescence with Var DNA, clearly differentiating the two. These results suggest that one-pot assay coupling LAMP with Cas12a can overcome limitation of LAMP to differentiation SNPs in the testing samples.

FIGS. 43A and 43B show example results of SNP detection by one-pot reaction coupling RT-LAMP with Cas12a-JP15 using synthetic gBlock NIR gene of Mpox (FIG. 43A) and Var (FIG. 43B). Reactions were performed the same as the reactions shown in FIGS. 42C and 42D except that guide RNA Mpox_crRNA1-9c10c was used instead of Mpox_crRNA1. The Mpox_crRNA1-9c10c spacer (UGUGCAAUCCUUGGACUUUG; e.g., nucleotides 21-40 of SEQ ID NO:31) has two mismatches to the target region of Mpox (TGTGCAATAATTGGACTTTG; e.g., nucleotides 310-329 of SEQ ID NO:47) on the 9^{th} and 10^{th} positions, and a single mismatch on the 10^{th} position compared to Var target region (TGTGCAATCATTGGACTTTG; e.g., nucleotides 310-329 of SEQ ID NO:48). In each figure, reactions with template (2000 copies/μl synthetic gBlock DNA per reaction) were depicted with circles and the corresponding NTC were shown as gray lines. The LAMP Cas12a-JP15 one-pot reactions compromise of LAMP and Cas 12a reagents in a single reaction mixture.

The one-pot assay with guide Mpox_crRNA1-9c10c showed background fluorescence with Mpox DNA and prominent fluorescence signal with Var DNA, giving a clear presence and absence of signal with the different templates. These results suggest that one-pot assay coupling LAMP with Cas12a can overcome limitation of LAMP to differentiation SNPs in the testing samples.

### BRIEF DESCRIPTION OF THE SEQUENCES

Some embodiments of this disclosure relate to the following provided sequences of example polynucleotides and/or example polypeptides.

SEQ ID NO: 1 is an example of a variant Cas12a, which may be referred to as Cas12a-
JP13.
MKKIDNFTNCYSLSKTLRFKAIPVGKTQENIDKKRLLEEDEKRAEDYKAVKKIIDRYHLSFINDVLNNVKLENLNEYAS
LENKSNRDDSENKELEKLEMNMRKEIAKAFKNNEEYKKLFKKEIIEEILPEFLEDEEEKEIVNSFKGFTTAFTGEHENR
ENMYSDEEKSTSIAYRCINENLPRFISNIKIFEKVKAILDEDEIEEINEEILNNDYSVEDFFTVDEFNEVLTQEGIDVY
NAIIGGIVTEDGTKIKGLNEYINLYNQKNKQRLPKLKPLYKQVLSERESMSFYAEGFTSDDEVLDALRNTLNKNSEIEN
AIEKLKKLFSNLDDYNLDGIYVKNGPAITTISNDVFGEWSVIRDKWNEEYDLIHMKKKAKDTEKYEEKRRKEYKKIESF
SIEELQELAGADLSIVEKIKEKISELIDEIKNAYSEAKNLFDADFTLEKKLKKDEKTVEIIKNLLDSVKDLEKYIKPEL
GTGKESNRDEVFYGEFTPAFDAISEIDNLYNKVRNYVTQKPYSKDKFKLYFQNPQFMGGWDRNKETDYRATILRKNGKY
YLAIMDKSNSKCLONIPESENDNYEKMNYKLIPGPSKMLPKVFFSKKYMDYYNPSEEILRIYKNGTFKKGDSENLNDCH
KLIDFYKDSISRHPDWSKSFDENESETEKYKDISGFYREVDEQGYKVSFEKVSKSEVDTLVEEGKLYLFQIYNKDESEK
SHGTPNLHTMYFKALFDENNHGNIRLCGGAEMEMRRASIKKEELVVHPANQPIKNKNPDNPKKTTTLPYDVYKDKRFSE
DQYELHIPISINKVPDNTFKINTEVRKLLRNDDNPYVIGIDRGERNLLYIVVVDGKGNIVEQYSLNEIINEYNGIKIRT
DYHSLLDKKEKERLEARONWKTIENIKELKEGYISQVVHKICELVEKYDAVIALEDLNSGFKNSRVKVEKQVYQKFEKM
LIDKLNYMVDKKTDPSASGGVLNGYQLTNKFESFKKMGTQNGIMFYIPAWLTSKIDPTTGFVNLLKTKYTSIAEAKKFI
SSFDSIRYDSDEDMFEFSIDYNNFPRTDADYRKKWEIYSYGDRIRIFRNPKKNNEFDYETVNLTEKFKELFDKYGINYS
SGDIREQLCAMSEKAFFEEFMGLLRLMLQMRNSITGRTDVDYLISPVKNSNGNFYDSRNYEKQESATLPKDADANGAYN
IARKVLWAIEQFKKAEEDKLDKVKIAISNKEWLEYAQTHCK*
SEQ ID NO: 2 is an example of a variant Cas12a, which may be referred to as Cas12a-
JP15.
MKKIDNFTNCYSVSKTLRFKAIPVGKTQENIDKKRLLEEDEKRAEDYKAVKKIIDRYHRSFIDKVLNNVKLDNLNEYAS
LFYKSNRDDSDNKKLEKLEAKMRKQIAKAFKNNEEYKKLFKKELIEEILPEFLEDEEEKEIVNSFKGFTTAFTGFHENR
ENMYSDEEKSTAIAYRCINENLPRFISNIKCFEKVKAILDEDEIEEINEEILNNDYSVEDFFTVDEFNEVLTQKGIDIY
NAIIGGIVTEDGTKIQGLNEYINLYNQQNKQRLPQLKPLYKQVLSERESMSFYAEGFTSDDEVLDALRDTLGKNSTIEN
AIEKLKKLFSNLDDYNLDGIYVKNGPAITTISNDVFGDWSVIRDKWNEEYDAVHSKKKAKDTEKYEEKRRKEYKKIESE
SIAELQELVDSDNSIVEKIKEKIKELIDEIKNAYSEAKNLFDSDFKQEKKLKKDEKTVELIKNLLDSVKDLEKYLKPFM
GTGKESNRDEVFYGEFTPCFDAISEIDNLYNKVRNYVTQKPYSTDKFKLYFQNPQFLGGWDRNKETDYRATILRKNGKY
YLAIMDKSNSKVFQNIPESDDDNYEKMNYKLIPGPSKMLPKVFFSKKNIDYFNPSEEILRIYKNGTFKKGDSENLDDCH
KLIDYFKDSISKHPDWSKSFDFKESETEKYKDISGFYREVDEQGYKVSFEKVSKSYVDTLVEEGKLYLFQIYNKDESEK
SHGTPNLHTMYFKALFDENNQGNIRLCGGAEMFMRRASIKKEELIVHPANQPIKNKNPLNPKKTTTLPYDVIKDKRFTE
DQYELHIPITINKVPDNAFKINHEVRKLLRNDDNPYVIGIDRGERNLLYIVVIDGKGNIVEQYSLNEIINEYNGIKIRT
DYHSLLDKKEKERLEARQNWKTIENIKELKEGYISQVVHKICELVEKYDAVIALEDLNSGFKNSRVKVEKQVYQKFEKM
LIDKLNYLVDKKTDPSENGGVLHGYQLTNKFESFKKMGTQNGIMFYIPAWLTSKIDPTTGFVDLLKPKYTSIAEAKKFI
SSFDSIRYNSDEDMFEFSIDYNKFPRTDADYRKKWTIYTHGDRIRTFRNPKKNNEWDNETVNLTEKFKKLFEKYGINYS
SGDLREQICAMSEKEFYKEFMGLLRLMLQMRNSITGRTDVDYLISPVKNSNGNFYDSRNYEKSATLPKDADANGAYNIA
RKVLWAIEQFKKAEDDKLDKVKIAISNKEWLEYAQTHCK*
SEQ ID NO: 3 is an example of a variant Cas12a, which may be referred to as Cas12a-
JP19.
MKKIDNFTNCYSLSKTLRFKLIPVGKTQENIDKKRLLEEDEKRAEDYKAVKKIIDRYHRSFIDDVLSSVKLENLNEYAE
LFYKSNKSDSDKKKMEKLESKMRKQIAKAFKSNEGYKKLFKKELIKEILPEFLEDEEEREIVESFKGFTTAFTGFYENR
KNMYSDEEKSTAIAYRCINENLPKFLDNVKCFEKVKAVLPKDEIEEINSDIQGLSGYSVEDVFSVDFFNFVLSQSGIDV
YNAIIGGYTTEDGTKIQGLNEYINLYNQQVKKSHRLPKLKPLYKQILSDRESVSFYPEKFESDDEVLEAIRDTYSNNSA
IYDTLEKLEKLFSNLDEYNTDGIYVKNGAAVTTISNSVFGDWSVIRDKWNEEYDSVHPKSKAKDTEKYEEKRKKAYKKI
ESFSIAELQKLADSSAKSIVEYFKSAIKELVDEIKEAYASAKDLLNSPYTEEKNLKKNDKAVELIKNLLDSVKELENFL
KQFMGTGKESNKDEVFYGDFTPCYDKLSQIDKLYDKVRNYVTQKPYSTDKFKLYFENPQFLGGWDRNKETDYRAVLLRK
DGQYYLAIMDKKHSRVFEKIPESDDEDCYEKMVYKLLPGPNKMLPKVFFSKKNIDTYNPPEEILKIYKKGTFKKGDSEN
LDDCHKFIDFFKDSIEKHPDWSYFDFKFSDTEEYKDISDFYREVEEQGYSISFEKVSESYIDELVEEGKLYLFQIYNKD
FSEHSKGTPNLHTMYFKMLFDENNLEDVVIKLNGGAEMEMRKASIKKDELIVHPANQPIKNKNPQNPKKQSTFEYDIIK
DKRFTEDQYSLHIPITINKKAENATNINDEVRKLLKDCDDNYVIGIDRGERNLLYICVIDGNGKIVEQYSLNEIINEYN
GIKYKTDYHKLLDKKEKERDEARKNWKTIENIKELKEGYISQVVHKICQLVEKYDAVIAMEDLNSGFKRSRVKVEKQVY
QKFEKMLIDKLNYLVDKKTDPDETGGLLHGYQLTNKFESFKKMGTQNGFIFYVPAWLTSKIDPTTGFVNLLKPKYTSVE
EAKEFISREDSIRYNADEDFFEFDIDYNKFSRTDADYRKKWTLCSYGDRIRTFRNPEKNNOWDNKTVTLTEEFKELFEK
YGIDYTSGDLKEQICSVSDADFYKKFMGLLRLTLQMRNSITGRTDVDYLISPVKNKNGTFYDSRNYDGQENATLPKDAD
ANGAYNIARKALWAIEQIKKAEDDELNKVKIAISNKEWLEYAQTSKK*
SEQ ID NO: 4 is an example of a variant Cas12a, which may be referred to as Cas12a-
JP16.
MSLNKFTNQYSLSKTLRFELKPIGKTLEHIQNKGLLSQDEQRAESYKKMKKTIDGFHKHFIELAMQNVKLTKLKEFADL
YNASAERKKDDEYKKELEKIQAELRKEIAEGFKTGAAKEIFSKLDKKELITELLENWIRTQEDEDIYFDESFKTFTTYF
GGFHENRKNMYTDKEQSTAIAYRLIHENLPKFLDNIRVEDKIKEIPELYEKLPLLYKEIKEYLNISSIDEAFSLDYENK
VLTQKQIDVYNLIIGGRTPEEGKKKIQGLNEYINLYNQQQKDKNNRIPKLKMLYKQILSDRESTSFLPEAFESSQEVLD
AINSYYHSNLISFQPEDKEEAENVLEKIKDLLTHLKDYDLNKIYLRNDTQLTHISQKLFGNYAVLGDALSFYYDQVLAP
SYQEDYQSANERKRKKLEKEKEKFLKQDYFSIAQLQNALDAYINSLDDTKDLKKNYTTNCIADYFHTHFKAEKKEDEDK
EFDLIANIEAKYSCVKGILENYPKDRKLHQDKKTIDDIKLFLDSLMELLHFVKPLILPSDSALEKDEAFYGQLEPWYDQ
LELLIPLYNKVRNYATQKPYSTEKFKLNFENSTLLNGWDVNKESDNTSVIFRKDGNYYLGIMDKKHNKIFKNVPKASTG
ESTYEKMVYKLLPGPNKMLPKVFFSDKNINYFAPSEEIQKIRKHGTHKKGEDENLNDCHKLIDFFKSSIEKHEDWKNFG
FQFSDTATYDDLSEFYREVEHOGYKITFTDIDENYINQLVDEGKLYLFQIYNKDFSPYSKGRPNLHTLYWKALFDPENL
KDVVYKLNGQAEVFYRKKSIKAENMVIHKAGEAIDNKNPLTTKKQSTFEYDLIKDKRYTVDKFQFHVPITLNFKASGKD
NINQEVLEYLKNNPDVNIIGIDRGERHLIYLTLIDQKGNILKQESLNTIVSERYNIETNYHELLAKREKERDKARKNWG
TIENIKELKEGYLSQVVHKIAKMMVEHNAIVVMEDLNFGFKRGRFKVEKQVYQKLEKMLIDKLNYLVFKDKSDNEPGGL
YKALQLTNKFESFKELGKQSGFLFYVPAWNTSKIDPTTGFVNLENTRYENIEKAREFFGKFESIRYNSDKGYFEFAFDY
NDFTTKAEGTRTDWTVCTHGERIKTFRNPEKNNOWDNKEIDLTEEFKDLFKKYGITYGDGKDIREQITSQTDKAFYERL
LHLFKLTLQMRNSKTGTDIDYLISPVMNAKGEFYDSRKADNTLPQDADANGAYHIAKKGLWLLQQINQAEDWKKLKLAI
SNKEWLRFVQGYKK*
SEQ ID NO: 5 is an example of a variant Cas12a, which may be referred to as Cas12a-
JP29.
MKSFDEFTNLYSLSKTLRFELKPVGKTLEHIEKNGIIEQDEQRAESYKKVKKIIDEYHREFIEEALSNVSLTKDNLEEY
AELYKKSKERKKDDKEKKELEEIQAELRKEIANCFKKNERFKNLFAKELITNELQSWVQDKEEINVIEKENNFTTYFTG
FHENRKNMYSDEDKSTAIAYRIIHENLPKFLDNIKVFQKIKEVYEKYELTLLEKELKSELGATSLDEIFSLDYFNKVLT
QKGIDRYNLIIGGRTTEDGEKIQGLNELINLYNQQQKDKNKRIPKLKPLYKQILSDRESTSFIPEAFENDNEVLEAIND
FYQEILIYRPEDKDKSENVLEKLKDLLQHLKDYDLDKIYIRNDKSITDISQKIFGDWSIIKSALKYYYDSKLATGKKKT
TQKQEKEKEKWLKQDYFSIAEIEDALSAYKNNAEDSKLSENPIADYFKKFMKNEKSEEEKEQDLFANIEAKYSSIKDLL
EKDYTKDKKLHQDKENVDKIKAFLDSIMELLHFVKPLILKSDSALEKDEAFYSEFEEFYEQLEQIIPLYNKVRNYMTQK
PYSTEKFKLNFENSTLLNGWDANKESDNTSVILRKDGKYYLGIMNKKHNKIFKNVPKAGSEAYYEKMVYKLLPGPNKML
PKVFFSKKNIDYFKPSQEILKIRNSGSHKKGDDENLEDCHKLIDFFKECIEKHPDWKNFDFQFSPTESYEDISEFYREV
EHOGYKITFTKIPENYIDQLVDEGKLYLFQIYNKDFSPHSKGKPNLHTLYWKALFDPENLKDVVYKLNGEAEVFYRKKS
IKKEEKIVHKAGKPIPNKNHHNSKKQSKFDYDIIKDRRYTEDKFLFHVPITLNFKAKGKNNINQEVNEFLKNNEDVHII
GIDRGERHLLYLSLINQKGNIIEQGSLNTITNEHYNYEVDYHEMLDKREKERDKARKNWKTIENIKELKEGYLSQVVHK
IAKMMVEHNAIVVMEDLNFGFKRGRFKVEKQVYQKFEKMLIDKLNYLVFKDKKPNEPGGVLKAYQLTSKFESFKKLGKQ
SGFLFYVPAAYTSKIDPTTGFVNLLHPKYENIEKAKEFFNKFESIRYNSNKDYFEFSFDYNKETTKAEGKKTDWTVCTH
GERFKTVRNNNGQWDSKEVDVTEELKNLFNEYGINYEDGKDIKEQITKTTNKKFFKRLLHLLKLTLQMRHSNTDSEEDY
ILSPVKNEKGEFFDSRKADDTLPMDADANGAYHIALKGLLLLQQIKQAEDLKKLNLWISNKEWLQFVONNKR*
SEQ ID NO: 6 is an example of a variant Cas12a, which may be referred to as Cas12a-
JP31.
MKTFDDFTNLYSLSKTLRFELIPVGKTLEHIEKKGLIEEDEKRAENYQKVKKIIDRYHKYFIEQALNNVKLDDLEEYQT
LYHKKKKDDNQKKEFEKIQEKLRKQIADAFKSNERFKKLFKKELIKELLPEFVQEEEERELVESFKNFTTYFTGFHENR
KNMYSDEEKSTAIAYRLIHENLPKFLDNMKIFEKIKAAPPKEKIEELYKDLEEYLNVTSIEEVFSLDYFNEVLTQKGID
VYNTIIGGRTAEEGKTKIQGLNEYINLYNQQQKKNKRLPKLKPLYKQILSDRESTSFIVEQFENDQEVLEAIEEFYQEL
IASYEGKGETVNVLETLKELLSNLSEYDLDKIYLRNDKSLTDISQKIFGDWSVIQNALSEYYDKVIPGKKKKDTEKYEE
KRKKKFKKQDYFSIAELQTALDTYEKEKYSTNSIVDYFATLGNESEKEFNLVEKIENAYSSVKDLLNTPYPEDKNLHQD
KESVEKIKNFLDSIMDLLHELKPLMATEETLEKDQTFYGEFEPLFEELSQIIPLYNKVRNYVTQKPYSTEKFKLNFENP
TLLDGWDKNKETDNTGVLFRKDGQYYLGIMDKKHNRVFENIPEPNDDDCYEKMEYKLLPGPSKMLPKVFFSKSNIDYEN
PSEEILRIYNHGTHKKGENENLEDCHQLIDFFKESINKHPDWKNFGFKESPTKQYESISEFYREVEEQGYKISFTKISE
SYIDQLVEEGKLYLFQIYNKDFSPHSKGKPNLHTLYWKALFDEENLKDVVYKLNGQAEVFYRKASIKKENKIVHPANQA
IANKNPLNKKKQSVFEYDIIKDKRFTVDKFQFHVPITLNFKATGSDNINQEVNEYLRONPDVHIIGIDRGERHLIYLTL
IDQKGNIIEQESLNTITNEHHTIRTPYHELLDKKEKERDEARKSWKTIENIKELKEGYLSQVVHKIAKLMVKYNAIVVM
EDLNFGFKRGRFKVEKQVYQKLEKMLIDKLNYLVFKDVEPDEPGGLLHALQLTNKFESFKKMGKQSGFLFYVPAWNTSK
IDPVTGFVNLLHPKYENVEKAKEFFSKFDSIRYNPEKDYFEFAFDYNNFTTKAEGTRTKWTICTYGERIKTFRNPEKNN
QWDNRTVNLTEEFKKLFEEYGIDYSNGGDLKEQICEQNDKDFFKKFMNLLKLTLQMRNSITGTEVDYLISPVANTQGEF
FDSRNADESLPQDADANGAYHIALKGLWVIEQIKQADDLKKIKLAISNKEWLQFVONRNK*
SEQ ID NO: 7 is an example of a variant Cas12a, which may be referred to as Cas12a-
JP1, wherein amino acids shown in bold (aa98-104, which correspond to 82-88 from SEQ ID
NO: 74) substituted amino acids 98-117 (underlined in SEQ ID NO: 17) of SEQ ID NO: 17.
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFRKKTRTENKEKEINNLRKAYYKEIKSLLDKKAEEWKEIYKKKEIQFNETDLKQKGTDELMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLFKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKENEEGIKIFWGYTEDLEKQAENLNSESRKKEEIILVKNYCDATLRIYQMMKYFALEAKKQDDI
PLAADCSPEFYNRFDEYYKDFKFIRYYDAIRNFVTKKPSNEDKIKLNFESGSLLTGFDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLFSDKNIQKPVFRLGANAEVFYRPASVRKEMDKERSKGGKEIIKYKRYTEDKMEL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGGIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRVIKENNNSTGYWEYKPIDLNEEFEDLFKKYGINEKSSDILSEIKE
LIKNNEGKLTRKQEFDGKNKNFYERFVYLFNLLLETRNTMSLRVKLDKRGNEIKLDEIDYGVDFFASPVKPFFTTAGVR
FVGRQIEGGKIQKEKKEEFTIKNFSDFERLFKNCPSDGFDSDGVGAYNIARKGIMILERIKONPKNPDLYISKDDWDSF
VLRNLS
SEQ ID NO: 8 is an example of a variant Cas12a, which may be referred to as Cas12a-
JP47 (N_ET-SSB_linker_Cas12a), wherein an ET-SSB domain is shown in bold (aa1-aa148)
and a linker sequence is underlined (aa149-aa180).
MEEKVGNLKPNMESVNVTVRVLEASEARQIQTKNGVRTISEAIVGDETGRVKLTLWGKHAGSIKEGQVVKIENAWTTAF
KGQVQLNAGSKTKIAEASEDGFPESSQIPENTPTAPQQMRGGGRGFRGGGRRYGRRGGRRQENEEGEEESGGSSGGSSG
SETPGTSESATPESSGGSSGGSKVNNGFSSLCVWSDFTRKFSLSKTLRFELKPVGRTEDFLKQNKVFEKDKTIDDSYNQ
AKFYFDKLHQKFINESLSPSSDSSNLKNIDLEYFAKQFrkktrteNKEKEINNLRKAYYKEIKSLLDKKAEEWKEIYKK
KEIQFNETDLKQKGTDFLMKSGILGILKYEFPKEKEEELKSKDWPSLFVEDKANPGDKVYIFDSFDDFATYLIKEQETR
KNLYKDDGTSTAVATRVISNFEKFLNNKKIFKDKYINYWKQIGITEGEKQIFEIDYYYNCFIQSGIDNYNDLIGKINQK
SKQYRDKNKIEKSKLPLFKVLDKQILGEVIKERELIIKTETETEEEVFINRFKEFIDONKQRILRAQNLMNDLINEEFE
NEYSGIYLKNSAINTIANRWFKNTTEFLLKLPQASKSKENKESPKVEPFVSLLDIKNALDDFEKEKDSLGTIFKDKYYK
TKESDEAPLNSDSQESYWKQFLKIWGYEFNQLFEDKENEEGIKIFWGYTEDLEKQAENLNSESRKKEEIILVKNYCDAT
LRIYQMMKYFALEAKKQDDIPLAADCSPEFYNRFDEYYKDFKFIRYYDAIRNFVTKKPSNEDKIKLNFESGSLLTGFDK
NKESEKLGIILKNKNNNKYFLGIINKKHNKIFESKNENEFIGNPAKDLYEKMELKLFPDPKKMIPKIAFADKNKKDFGW
TQEIQKIKEDFGNFQENKKDSKDLKFDKNKLSKLIEYYQNCLEKGNYKKEFDFEWKKPEEYQSMSEFNQDIEKKNYKIK
FIPIKASYIDDKVKNGELYLFEISNKDFIWPNQKKNIHTLYFLNLESDKNIQKPVERLGANAEVFYRPASVRKEMDKER
SKGGKEIIKYKRYTEDKMFLHLPIEINYGCPKAPNKNQYNKKIIEFLNKNKDEINIIGIDRGEKNLLYYTVINQKGEIL
DHGSLNEINGVNYFEKLIEREKERQINRQSWEPVVKIKDLKKGYLSYIVRKIADLVEKYNAIIVLEDLNMRFKQVRGGI
ERSIYQQFEKQLIDKLGYLVFKDDRGPESPGGVLNGYQLLAPFTTEKDLGKQTGIIFYTNAEYTSKTDPITGYRKNIYI
SNSASQKKIKENLINKLKEIGWDEKENSYFFTYNQKDFGSPISKEWTLYSKVPRVIKENNNSTGYWEYKPIDLNEEFED
LFKKYGINEKSSDILSEIKELIKNNEGKLTRKQEFDGKNKNFYERFVYLENLLLETRNTMSLRVKLDKRGNEIKLDEID
YGVDFFASPVKPFFTTAGVRFVGRQIEGGKIQKEKKEEFTIKNFSDFERLFKNCPSDGFDSDGVGAYNIARKGIMILER
IKQNPKNPDLYISKDDWDSFVLRNLS
SEQ ID NO: 9 is an example of a variant Cas12a, which may be referred to as Cas12a-
JP48 (Cas12a_linker_ET_SSB), wherein a linker sequence is underlined (aa1349-aa1380)
and an ET-SSB domain is shown in bold (aa1381-aa1527).
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFrkktrteNKEKEINNLRKAYYKEIKSLLDKKAEEWKEIYKKKEIQFNETDLKQKGTDFLMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLFKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKENEEGIKIFWGYTEDLEKQAENLNSFSRKKEEIILVKNYCDATLRIYQMMKYFALEAKKQDDI
PLAADCSPEFYNRFDEYYKDFKFIRYYDAIRNFVTKKPSNEDKIKLNFESGSLLTGFDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLFSDKNIQKPVERLGANAEVFYRPASVRKEMDKERSKGGKEIIKYKRYTEDKMEL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGGIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRVIKENNNSTGYWEYKPIDLNEEFEDLFKKYGINEKSSDILSEIKE
LIKNNEGKLTRKQEFDGKNKNFYERFVYLENLLLETRNTMSLRVKLDKRGNEIKLDEIDYGVDFFASPVKPFFTTAGVR
FVGRQIEGGKIQKEKKEEFTIKNFSDFERLFKNCPSDGFDSDGVGAYNIARKGIMILERIKONPKNPDLYISKDDWDSF
VLRNLSGGSSGGSSGSETPGTSESATPESSGGSSGGSEEKVGNLKPNMESVNVTVRVLEASEARQIQTKNGVRTISEAI
VGDETGRVKLTLWGKHAGSIKEGQVVKIENAWTTAFKGQVOLNAGSKTKIAEASEDGFPESSQIPENTPTAPQQMRGGG
RGFRGGGRRYGRRGGRRQENEEGEEE
SEQ ID NO: 10 is an example of a variant Cas12a, which may be referred to as
Cas12a-JP45 (Cas12a_linker_Sso7d), wherein a linker sequence is underlined (aa1349-
aa1380) and an Sso7d domain is shown in bold (aa1381-aa1443).
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFrkktrteNKEKEINNLRKAYYKEIKSLLDKKAEEWKEIYKKKEIQFNETDLKQKGTDELMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLFKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKFNEEGIKIFWGYTEDLEKQAENLNSFSRKKEEIILVKNYCDATLRIYQMMKYFALEAKKQDDI
PLAADCSPEFYNRFDEYYKDFKFIRYYDAIRNFVTKKPSNEDKIKLNFESGSLLTGEDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLESDKNIQKPVERLGANAEVFYRPASVRKEMDKERSKGGKEIIKYKRYTEDKMEL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGGIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRVIKENNNSTGYWEYKPIDLNEEFEDLFKKYGINEKSSDILSEIKE
LIKNNEGKLTRKQEFDGKNKNFYERFVYLENLLLETRNTMSLRVKLDKRGNEIKLDEIDYGVDFFASPVKPFFTTAGVR
FVGRQIEGGKIQKEKKEEFTIKNFSDFERLFKNCPSDGFDSDGVGAYNIARKGIMILERIKONPKNPDLYISKDDWDSF
VLRNLSGGSSGGSSGSETPGTSESATPESSGGSSGGSATVKFKYKGEEKEVDISKIKKVWRVGKMISFTYDEGGGKTGR
GAVSEKDAPKELLOMLEKOKK
SEQ ID NO: 11 is an example of a variant Cas12a, which may be referred to as
Cas12a-JP42 (G1019K), wherein the variant Cas12a comprises a G1019K substitution
(relative to SEQ ID NO: 17).
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFrkktrteNKEKEINNLRKAYYKEIKSLLDKKAEEWKEIYKKKEIQFNETDLKQKGTDELMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLFKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKENEEGIKIFWGYTEDLEKQAENLNSESRKKEEIILVKNYCDATLRIYQMMKYFALEAKKQDDI
PLAADCSPEFYNRFDEYYKDFKFIRYYDAIRNFVTKKPSNEDKIKLNFESGSLLTGFDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLFSDKNIQKPVFRLGANAEVFYRPASVRKEMDKERSKGGKEIIKYKRYTEDKMEL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGKIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRVIKENNNSTGYWEYKPIDLNEEFEDLFKKYGINEKSSDILSEIKE
LIKNNEGKLTRKQEFDGKNKNFYERFVYLFNLLLETRNTMSLRVKLDKRGNEIKLDEIDYGVDFFASPVKPFFTTAGVR
FVGRQIEGGKIQKEKKEEFTIKNFSDFERLFKNCPSDGFDSDGVGAYNIARKGIMILERIKONPKNPDLYISKDDWDSF
VLRNLS
SEQ ID NO: 12 is an example of a variant Cas12a, which may be referred to as
Cas12a-JP52 (I1155R), wherein the variant Cas12a comprises an I1155R substitution
(relative to SEQ ID NO: 17).
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFrkktrteNKEKEINNLRKAYYKEIKSLLDKKAEEWKEIYKKKEIQFNETDLKQKGTDFLMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLEKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKENEEGIKIFWGYTEDLEKQAENLNSFSRKKEEIILVKNYCDATLRIYQMMKYFALEAKKODDI
PLAADCSPEFYNRFDEYYKDFKFIRYYDAIRNFVTKKPSNEDKIKLNFESGSLLTGFDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLESDKNIQKPVERLGANAEVFYRPASVRKEMDKERSKGGKEIIKYKRYTEDKMEL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGGIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRV&KENNNSTGYWEYKPIDLNEEFEDLFKKYGINEKSSDILSEIKE
LIKNNEGKLTRKQEFDGKNKNFYERFVYLENLLLETRNTMSLRVKLDKRGNEIKLDEIDYGVDFFASPVKPFFTTAGVR
FVGRQIEGGKIQKEKKEEFTIKNFSDFERLFKNCPSDGEDSDGVGAYNIARKGIMILERIKONPKNPDLYISKDDWDSF
VLRNLS
SEQ ID NO: 13 is an example of a variant Cas12a, which may be referred to as
Cas12a-JP53 (G1019KI1155R), wherein the variant Cas12a comprises a G1019K
substitution (relative to SEQ ID NO: 17) and an I1155R substitution (relative to SEQ ID
NO: 17).
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFrkktrteNKEKEINNLRKAYYKEIKSLLDKKAEEWKEIYKKKEIQFNETDLKQKGTDFLMK
SGILGILKYEFPKEKEEELKSKDWPSLFVEDKANPGDKVYIFDSFDDFATYLIKFQETRKNLYKDDGTSTAVATRVISN
FEKFLNNKKIFKDKYINYWKQIGITEGEKQIFEIDYYYNCFIQSGIDNYNDLIGKINQKSKQYRDKNKIEKSKLPLEKV
LDKQILGEVIKERELIIKTETETEEEVFINRFKEFIDONKORILRAQNLMNDLINEEFENEYSGIYLKNSAINTIANRW
FKNTTEFLLKLPQASKSKENKESPKVEPFVSLLDIKNALDDFEKEKDSLGTIFKDKYYKTKESDEAPLNSDSQESYWKQ
FLKIWGYEFNQLFEDKENEEGIKIFWGYTEDLEKQAENLNSESRKKEEIILVKNYCDATLRIYQMMKYFALEAKKQDDI
PLAADCSPEFYNRFDEYYKDFKFIRYYDAIRNFVTKKPSNEDKIKLNFESGSLLTGFDKNKESEKLGIILKNKNNNKYF
LGIINKKHNKIFESKNENEFIGNPAKDLYEKMELKLFPDPKKMIPKIAFADKNKKDFGWTQEIQKIKEDEGNFQENKKD
SKDLKFDKNKLSKLIEYYQNCLEKGNYKKEFDFEWKKPEEYQSMSEFNQDIEKKNYKIKFIPIKASYIDDKVKNGELYL
FEISNKDFIWPNQKKNIHTLYFLNLFSDKNIQKPVFRLGANAEVFYRPASVRKEMDKERSKGGKEIIKYKRYTEDKMFL
HLPIEINYGCPKAPNKNQYNKKIIEFLNKNKDEINIIGIDRGEKNLLYYTVINQKGEILDHGSLNEINGVNYFEKLIER
EKERQINRQSWEPVVKIKDLKKGYLSYIVRKIADLVEKYNAIIVLEDLNMRFKQVRGEIERSIYQQFEKQLIDKLGYLV
FKDDRGPESPGGVLNGYQLLAPFTTFKDLGKQTGIIFYTNAEYTSKTDPITGYRKNIYISNSASQKKIKENLINKLKEI
GWDEKENSYFFTYNQKDFGSPISKEWTLYSKVPRVRKENNNSTGYWEYKPIDLNEEFEDLFKKYGINEKSSDILSEIKE
LIKNNEGKLTRKQEFDGKNKNFYERFVYLENLLLETRNTMSLRVKLDKRGNEIKLDEIDYGVDFFASPVKPFFTTAGVR
FVGRQIEGGKIQKEKKEEFTIKNFSDFERLFKNCPSDGEDSDGVGAYNIARKGIMILERIKONPKNPDLYISKDDWDSF
VLRNLS
SEQ ID NO: 14 is an example of a variant Cas12a, which may be referred to as
Cas12a-JP54 (N_ET-SSB_linker_Cas12a with I1155R), wherein an ET-SSB domain is
shown in bold (aa1-aa148) and a linker sequence is underlined (aa149-aa180) and wherein
the variant Cas12a comprises an I1155R substitution (relative to SEQ ID NO: 17).
MEEKVGNLKPNMESVNVTVRVLEASEARQIQTKNGVRTISEAIVGDETGRVKLTLWGKHAGSIKEGQVVKIENAWTTAF
KGQVQLNAGSKTKIAEASEDGFPESSQIPENTPTAPQQMRGGGRGFRGGGRRYGRRGGRRQENEEGEEESGGSSGGSSG
SETPGTSESATPESSGGSSGGSKVNNGFSSLCVWSDFTRKFSLSKTLRFELKPVGRTEDFLKQNKVFEKDKTIDDSYNQ
AKFYFDKLHQKFINESLSPSSDSSNLKNIDLEYFAKQFrkktrteNKEKEINNLRKAYYKEIKSLLDKKAEEWKEIYKK
KEIQFNETDLKQKGTDFLMKSGILGILKYEFPKEKEEELKSKDWPSLFVEDKANPGDKVYIFDSFDDFATYLIKEQETR
KNLYKDDGTSTAVATRVISNFEKFLNNKKIFKDKYINYWKQIGITEGEKQIFEIDYYYNCFIQSGIDNYNDLIGKINQK
SKQYRDKNKIEKSKLPLFKVLDKQILGEVIKERELIIKTETETEEEVFINRFKEFIDONKORILRAQNLMNDLINEEFE
NEYSGIYLKNSAINTIANRWFKNTTEFLLKLPQASKSKENKESPKVEPFVSLLDIKNALDDFEKEKDSLGTIFKDKYYK
TKESDEAPLNSDSQESYWKQFLKIWGYEFNQLFEDKENEEGIKIFWGYTEDLEKQAENLNSESRKKEEIILVKNYCDAT
LRIYQMMKYFALEAKKQDDIPLAADCSPEFYNRFDEYYKDFKFIRYYDAIRNFVTKKPSNEDKIKLNFESGSLLTGEDK
NKESEKLGIILKNKNNNKYFLGIINKKHNKIFESKNENEFIGNPAKDLYEKMELKLFPDPKKMIPKIAFADKNKKDFGW
TQEIQKIKEDFGNFQENKKDSKDLKFDKNKLSKLIEYYQNCLEKGNYKKEFDFEWKKPEEYQSMSEFNQDIEKKNYKIK
FIPIKASYIDDKVKNGELYLFEISNKDFIWPNQKKNIHTLYFLNLFSDKNIQKPVERLGANAEVFYRPASVRKEMDKER
SKGGKEIIKYKRYTEDKMFLHLPIEINYGCPKAPNKNQYNKKIIEFLNKNKDEINIIGIDRGEKNLLYYTVINQKGEIL
DHGSLNEINGVNYFEKLIEREKERQINRQSWEPVVKIKDLKKGYLSYIVRKIADLVEKYNAIIVLEDLNMRFKQVRGGI
ERSIYQQFEKQLIDKLGYLVFKDDRGPESPGGVLNGYQLLAPFTTFKDLGKQTGIIFYTNAEYTSKTDPITGYRKNIYI
SNSASQKKIKENLINKLKEIGWDEKENSYFFTYNQKDFGSPISKEWTLYSKVPRV&KENNNSTGYWEYKPIDLNEEFED
LFKKYGINEKSSDILSEIKELIKNNEGKLTRKQEFDGKNKNFYERFVYLFNLLLETRNTMSLRVKLDKRGNEIKLDEID
YGVDFFASPVKPFFTTAGVRFVGRQIEGGKIQKEKKEEFTIKNFSDFERLFKNCPSDGFDSDGVGAYNIARKGIMILER
IKQNPKNPDLYISKDDWDSFVLRNLS
SEQ ID NO: 15 is an example of a variant Cas12a, which may be referred to as
Cas12a-JP51 (N_ET-SSB_linker_Cas12a with G1019K), wherein an ET-SSB domain is
shown in bold (aa1-aa148) and a linker sequence is underlined (aa149-aa180) and wherein
the variant Cas12a comprises a G1019K substitution (relative to SEQ ID NO: 17).
MEEKVGNLKPNMESVNVTVRVLEASEARQIQTKNGVRTISEAIVGDETGRVKLTLWGKHAGSIKEGQVVKIENAWTTAF
KGQVOLNAGSKTKIAEASEDGFPESSQIPENTPTAPQQMRGGGRGFRGGGRRYGRRGGRRQENEEGEEESGGSSGGSSG
SETPGTSESATPESSGGSSGGSKVNNGFSSLCVWSDFTRKFSLSKTLRFELKPVGRTEDFLKQNKVFEKDKTIDDSYNQ
AKFYFDKLHQKFINESLSPSSDSSNLKNIDLEYFAKQFrkktrteNKEKEINNLRKAYYKEIKSLLDKKAEEWKEIYKK
KEIQFNETDLKQKGTDFLMKSGILGILKYEFPKEKEEELKSKDWPSLFVEDKANPGDKVYIFDSFDDFATYLIKEQETR
KNLYKDDGTSTAVATRVISNFEKFLNNKKIFKDKYINYWKQIGITEGEKQIFEIDYYYNCFIQSGIDNYNDLIGKINQK
SKQYRDKNKIEKSKLPLFKVLDKQILGEVIKERELIIKTETETEEEVFINRFKEFIDONKORILRAQNLMNDLINEEFE
NEYSGIYLKNSAINTIANRWFKNTTEFLLKLPQASKSKENKESPKVEPFVSLLDIKNALDDFEKEKDSLGTIFKDKYYK
TKESDEAPLNSDSQESYWKQFLKIWGYEFNQLFEDKENEEGIKIFWGYTEDLEKQAENLNSESRKKEEIILVKNYCDAT
LRIYQMMKYFALEAKKQDDIPLAADCSPEFYNRFDEYYKDFKFIRYYDAIRNFVTKKPSNEDKIKLNFESGSLLTGEDK
NKESEKLGIILKNKNNNKYFLGIINKKHNKIFESKNENEFIGNPAKDLYEKMELKLFPDPKKMIPKIAFADKNKKDFGW
TQEIQKIKEDFGNFQENKKDSKDLKFDKNKLSKLIEYYQNCLEKGNYKKEFDFEWKKPEEYQSMSEFNQDIEKKNYKIK
FIPIKASYIDDKVKNGELYLFEISNKDFIWPNQKKNIHTLYFLNLFSDKNIQKPVERLGANAEVFYRPASVRKEMDKER
SKGGKEIIKYKRYTEDKMFLHLPIEINYGCPKAPNKNQYNKKIIEFLNKNKDEINIIGIDRGEKNLLYYTVINQKGEIL
DHGSLNEINGVNYFEKLIEREKERQINRQSWEPVVKIKDLKKGYLSYIVRKIADLVEKYNAIIVLEDLNMRFKQVRGKI
ERSIYQQFEKQLIDKLGYLVFKDDRGPESPGGVLNGYQLLAPFTTFKDLGKQTGIIFYTNAEYTSKTDPITGYRKNIYI
SNSASQKKIKENLINKLKEIGWDEKENSYFFTYNQKDFGSPISKEWTLYSKVPRVIKENNNSTGYWEYKPIDLNEEFED
LFKKYGINEKSSDILSEIKELIKNNEGKLTRKQEFDGKNKNFYERFVYLFNLLLETRNTMSLRVKLDKRGNEIKLDEID
YGVDFFASPVKPFFTTAGVRFVGRQIEGGKIQKEKKEEFTIKNFSDFERLFKNCPSDGFDSDGVGAYNIARKGIMILER
IKQNPKNPDLYISKDDWDSFVLRNLS
SEQ ID NO: 16 is an example of a variant Cas12a, which may be referred to as
Cas12a-JP55 (N_ET-SSB_linker_Cas12a with G1019KI1155R), wherein an ET-SSB
domain is shown in bold (aa1-aa148) and a linker sequence is underlined (aa149-aa180) and
wherein the variant Cas12a comprises a G1019K substitution (relative to SEQ ID NO: 17)
and an I1155R substitution (relative to SEQ ID NO: 17).
MEEKVGNLKPNMESVNVTVRVLEASEARQIQTKNGVRTISEAIVGDETGRVKLTLWGKHAGSIKEGQVVKIENAWTTAF
KGQVOLNAGSKTKIAEASEDGFPESSQIPENTPTAPQQMRGGGRGFRGGGRRYGRRGGRRQENEEGEEESGGSSGGSSG
SETPGTSESATPESSGGSSGGSKVNNGFSSLCVWSDFTRKFSLSKTLRFELKPVGRTEDFLKQNKVFEKDKTIDDSYNQ
AKFYFDKLHQKFINESLSPSSDSSNLKNIDLEYFAKQFrkktrteNKEKEINNLRKAYYKEIKSLLDKKAEEWKEIYKK
KEIQFNETDLKQKGTDFLMKSGILGILKYEFPKEKEEELKSKDWPSLFVEDKANPGDKVYIFDSFDDFATYLIKEQETR
KNLYKDDGTSTAVATRVISNFEKFLNNKKIFKDKYINYWKQIGITEGEKQIFEIDYYYNCFIQSGIDNYNDLIGKINQK
SKQYRDKNKIEKSKLPLFKVLDKQILGEVIKERELIIKTETETEEEVFINRFKEFIDONKORILRAQNLMNDLINEEFE
NEYSGIYLKNSAINTIANRWFKNTTEFLLKLPQASKSKENKESPKVEPFVSLLDIKNALDDFEKEKDSLGTIFKDKYYK
TKESDEAPLNSDSQESYWKQFLKIWGYEFNQLFEDKENEEGIKIFWGYTEDLEKQAENLNSESRKKEEIILVKNYCDAT
LRIYQMMKYFALEAKKQDDIPLAADCSPEFYNRFDEYYKDFKFIRYYDAIRNFVTKKPSNEDKIKLNFESGSLLTGEDK
NKESEKLGIILKNKNNNKYFLGIINKKHNKIFESKNENEFIGNPAKDLYEKMELKLFPDPKKMIPKIAFADKNKKDFGW
TQEIQKIKEDFGNFQENKKDSKDLKFDKNKLSKLIEYYQNCLEKGNYKKEFDFEWKKPEEYQSMSEFNQDIEKKNYKIK
FIPIKASYIDDKVKNGELYLFEISNKDFIWPNQKKNIHTLYFLNLFSDKNIQKPVERLGANAEVFYRPASVRKEMDKER
SKGGKEIIKYKRYTEDKMFLHLPIEINYGCPKAPNKNQYNKKIIEFLNKNKDEINIIGIDRGEKNLLYYTVINQKGEIL
DHGSLNEINGVNYFEKLIEREKERQINRQSWEPVVKIKDLKKGYLSYIVRKIADLVEKYNAIIVLEDLNMRFKQVRGEI
ERSIYQQFEKQLIDKLGYLVFKDDRGPESPGGVLNGYQLLAPFTTFKDLGKQTGIIFYTNAEYTSKTDPITGYRKNIYI
SNSASQKKIKENLINKLKEIGWDEKENSYFFTYNQKDFGSPISKEWTLYSKVPRVKENNNSTGYWEYKPIDLNEEFED
LFKKYGINEKSSDILSEIKELIKNNEGKLTRKQEFDGKNKNFYERFVYLENLLLETRNTMSLRVKLDKRGNEIKLDEID
YGVDFFASPVKPFFTTAGVRFVGRQIEGGKIQKEKKEEFTIKNFSDFERLFKNCPSDGFDSDGVGAYNIARKGIMILER
IKQNPKNPDLYISKDDWDSFVLRNLS
SEQ ID NO: 17 is an example of a wild type Cas12a, which may be referred to as
YmeCas12a. The underlined sequence (aa98-aa117) was changed to amino acids 98-104
(bold in SEQ ID NO: 7) of SEQ ID NO: 7.
MSKVNNGFSSLCVWSDFTRKFSLSKTLRFELKPVGRTEDFLKQNKVFEKDKTIDDSYNQAKFYFDKLHQKFINESLSPS
SDSSNLKNIDLEYFAKQFLKLKSEIQKLKGEKKOKEANNKEKEINNLRKAYYKEIKSLLDKKAEEWKEIYKKKEIQFNE
TDLKQKGTDFLMKSGILGILKYEFPKEKEEELKSKDWPSLFVEDKANPGDKVYIFDSFDDFATYLIKFQETRKNLYKDD
GTSTAVATRVISNFEKFLNNKKIFKDKYINYWKQIGITEGEKQIFEIDYYYNCFIQSGIDNYNDLIGKINQKSKQYRDK
NKIEKSKLPLFKVLDKQILGEVIKERELIIKTETETEEEVFINRFKEFIDONKORILRAQNLMNDLINEEFENEYSGIY
LKNSAINTIANRWFKNTTEFLLKLPQASKSKENKESPKVEPFVSLLDIKNALDDFEKEKDSLGTIFKDKYYKTKESDEA
PLNSDSQESYWKQFLKIWGYEFNQLFEDKFNEEGIKIFWGYTEDLEKQAENLNSESRKKEEIILVKNYCDATLRIYQMM
KYFALEAKKQDDIPLAADCSPEFYNRFDEYYKDFKFIRYYDAIRNFVTKKPSNEDKIKLNFESGSLLTGEDKNKESEKL
GIILKNKNNNKYFLGIINKKHNKIFESKNENEFIGNPAKDLYEKMELKLFPDPKKMIPKIAFADKNKKDFGWTQEIQKI
KEDFGNFQENKKDSKDLKFDKNKLSKLIEYYQNCLEKGNYKKEFDFEWKKPEEYQSMSEFNQDIEKKNYKIKFIPIKAS
YIDDKVKNGELYLFEISNKDFIWPNQKKNIHTLYFLNLFSDKNIQKPVERLGANAEVFYRPASVRKEMDKERSKGGKEI
IKYKRYTEDKMFLHLPIEINYGCPKAPNKNQYNKKIIEFLNKNKDEINIIGIDRGEKNLLYYTVINQKGEILDHGSLNE
INGVNYFEKLIEREKERQINRQSWEPVVKIKDLKKGYLSYIVRKIADLVEKYNAIIVLEDLNMRFKQVRGGIERSIYQQ
FEKQLIDKLGYLVFKDDRGPESPGGVLNGYQLLAPFTTFKDLGKQTGIIFYTNAEYTSKTDPITGYRKNIYISNSASQK
KIKENLINKLKEIGWDEKENSYFFTYNQKDFGSPISKEWTLYSKVPRVIKENNNSTGYWEYKPIDLNEEFEDLEKKYGI
NEKSSDILSEIKELIKNNEGKLTRKQEFDGKNKNFYERFVYLFNLLLETRNTMSLRVKLDKRGNEIKLDEIDYGVDFFA
SPVKPFFTTAGVRFVGRQIEGGKIQKEKKEEFTIKNFSDFERLFKNCPSDGFDSDGVGAYNIARKGIMILERIKONPKN
PDLYISKDDWDSFVLRNLS
SEQ ID NO: 18 is an example of a guide RNA targeting the E gene of SARS-COV-2,
which may be referred to as crRNA1.
CAAUUUCUACUUUUGUAGAUGGAAGAGACAGGUACGUUAA
SEQ ID NO: 19 is an example of a guide RNA targeting the E gene of SARS-COV-2,
which may be referred to as crRNA2.
CAAUUUCUACUUUUGUAGAUUUGCUUUCGUGGUAUUCUUG
SEQ ID NO: 20 is an example of a guide RNA targeting the E gene of SARS-COV-2,
which may be referred to as crRNA3.
CAAUUUCUACUUUUGUAGAUCAAGACUCACGUUAACAAUA
SEQ ID NO: 21 is an example of a guide RNA targeting the E gene of SARS-COV-2,
which may be referred to as crRNA4.
CAAUUUCUACUUUUGUAGAUGUGGUAUUCUUGCUAGUUAC
SEQ ID NO: 22 is an example of a guide RNA targeting the E gene of SARS-COV-2,
which may be referred to as E_crRNA4-9g.
CAAUUUCUACUUUUGUAGAUGUGGUAUUgUUGCUAGUUAC
SEQ ID NO: 23 is an example of a guide RNA targeting the E gene of SARS-COV-2,
which may be referred to as E_crRNA4-15t.
CAAUUUCUACUUUUGUAGAUGUGGUAUUCUUGCUuGUUAC
SEQ ID NO: 24 is an example of a guide RNA targeting the E gene of SARS-COV-2,
which may be referred to as E_crRNA4_910ga.
CAAUUUCUACUUUUGUAGAUGUGGUAUUgaUGCUAGUUAC
SEQ ID NO: 25 is an example of a guide RNA targeting the E gene of SARS-COV-2,
which may be referred to as E_crRNA4_34cc.
CAAUUUCUACUUUUGUAGAUGUccUAUUCUUGCUuGUUAC
SEQ ID NO: 26 is an example of a guide RNA targeting the N1R gene of
Orthopoxviruses, which may be referred to as Mpox_crRNA1.
CAAUUUCUACUUUUGUAGAUUGUGCAAUAAUUGGACUUUG
SEQ ID NO: 27 is an example of a guide RNA targeting the N1R gene of
Orthopoxviruses, which may be referred to as Mpox_crRNA3.
CAAUUUCUACUUUUGUAGAUAUCAGAAAUGACUCCAUGAA
SEQ ID NO: 28 is an example of a guide RNA targeting the N1R gene of
Orthopoxviruses, which may be referred to as Mpox_crRNA4.
CAAUUUCUACUUUUGUAGAUUACUCAAUCAGCUAUUGUCA
SEQ ID NO: 29 is an example of a guide RNA targeting the N1R gene of
Orthopoxviruses, which may be referred to as Mpox_crRNA8.
CAAUUUCUACUUUUGUAGAUAAUGAUCUCCACGCAAUUGU
SEQ ID NO: 30 is an example of a guide RNA targeting the N1R gene of
Orthopoxviruses, which may be referred to as Mpox_crRNA9.
CAAUUUCUACUUUUGUAGAUAAGACUCUUCCAGUGACAAU
SEQ ID NO: 31 is an example of a guide RNA targeting the N1R gene of
Orthopoxviruses, which may be referred to as Mpox_crRNA1-9c10c.
CAAUUUCUACUUUUGUAGAUUGUGCAAUccUUGGACUUUG
SEQ ID NO: 32 is an example of a Cas12b guide RNA targeting the N gene of SARS-
CoV-2, which may be referred to as Cas12b guide.
GUCUAGAGGACAGAAUUUUUCAACGGGUGUGCCAAUGGCCACUUUCCAGGUGGCAAAGCCCGUUGAGCUUCUCAAAUCU
GAGAAGUGGCACCGAAGAACGCUGAAGCGCUG
SEQ ID NO: 33 is an example of a guide RNA targeting the N gene of SARS-COV-2,
which may be referred to as N2_RNA1.
CAAUUUCUACUUUUGUAGAUCCCCCAGCGCUUCAGCGUUC
SEQ ID NO: 34 is an example of a partial sequence of the E gene of SARS-COV-2,
which may be referred to as E1-PCR.
TTCGTTTCGGAAGAGACAGGTACGTTAATAGTTAATAGCGTACTTCTTTTTCTTGCTTTCGTGGTATTCTTGCTAGTTA
CACTAGCCATCCTTACTGCGCTTCGATTGTGTGCGTACTGCTGCAATATTGTTAACGTGAGTCTTGTAAAACCT
SEQ ID NO: 35 is an example of a PCRprimer to generate E1-PCR, which may be
referred to as E1-PCR-fwd.
TTCGTTTCGGAAGAGACAG
SEQ ID NO: 36 is an example of a partial sequence of the E gene of SARS-COV-2,
which may be referred to as E1-PCR-rev.
AGGTTTTACAAGACTCACGT
SEQ ID NO: 37 is an example of a trans substrate of Cas12a having a 5′ FAM label, a
first quencher (ZEN), and a second quencher (Iowa Black FQ) 3′ of the first quencher, which
may be referred to as NZ-GT reporter.
/FAM/ATGTCGTCAGTGTGTGTGTGTGACG/Zen/TG/IABKFQ/
SEQ ID NO: 38 is an example of a trans substrate of Cas12a having a 5′ FAM label
and a 3′ quencher (Iowa Black FQ), which may be referred to as T15 reporter.
/FAM/TTTTTTTTTTTTTTT/IABKFQ/
SEQ ID NO: 39 is an example of a trans substrate of Cas12a having a 5′ FAM label
and a 3′ quencher (Iowa Black FQ), which may be referred to as T5 reporter.
/FAM/TTTTT/IABKFQ/
SEQ ID NO: 40 is an example of a trans substrate of Cas12a having a 5′ FAM label
and a 3′ quencher (Iowa Black FQ), which may be referred to as 10C reporter.
/FAM/CCCCCCCCCC/IABKFQ/
SEQ ID NO: 41 is an example of a LAMP primer for the N1R gene of
Orthopoxviruses, which may be referred to as N1R_F3.
GAATTGATGCAATGGAGCTA
SEQ ID NO: 42 is an example of a LAMP primer for the N1R gene of
Orthopoxviruses, which may be referred to as N1R_B3.
GCAGCATAAGTAGTATGTCG
SEQ ID NO: 43 is an example of a LAMP primer for the N1R gene of
Orthopoxviruses, which may be referred to as N1R_FIP.
TCTCCACGCAATTGTCGATATTGGTAGCGAGTTGAAGGAGTT
SEQ ID NO: 44 is an example of a LAMP primer for the N1R N gene of
Orthopoxviruses, which may be referred to as N1R_BIP.
ACTCCATGAAAACCGCCAAAGAAGACTCTTCCAGTGACA
SEQ ID NO: 45 is an example of a LAMP primer for the N1R gene of
Orthopoxviruses, which may be referred to as N1R_LF.
CCACGGAAGTGAATTCGAG
SEQ ID NO: 46 is an example of a LAMP primer for the N1R gene of
Orthopoxviruses, which may be referred to as N1R_LB.
TGGACTTTGTACTCAATCAGCT
SEQ ID NO: 47 is and example of the gBlock for the N1R gene of Mpox, which may
be referred to as Mpox.
ATGGCCTCTCCTTGTGCCCAGTTCAGTCCCTGTCATTGCCACGCTACTAAGGACTCCCTGAATACCGTGACTGACGTCA
GACATTGTCTGACTGAATACATCCTGTGGGTTTCTCATAGATGGACCCATAGAGAAAGCGCAGGGCCTCTCTACAGGCT
TCTCATCTCTTTCAGAATTGATGCAATGGAGCTATTTGGTAGCGAGTTGAAGGAGTTCTCGAATTCACTTCCGTGGGAC
AATATCGACAATTGCGTGGAGATCATTAAATGTTTCATCAGAAATGACTCCATGAAAACCGCCAAAGAACTTTGTGCAA
TAATTGGACTTTGTACTCAATCAGCTATTGTCACTGGAAGAGTCTTCAATGATAAGTATATCGACATACTACTTATGCT
GCGAAAGATTCTGAACGAGAACGACTATCTCACCCTCTTGGATCATATCCTCACT
SEQ ID NO: 48 is and example of the gBlock for a N1R gene mimic of variola, which
may be referred to as Var.
ATGGCCTTTCCTTGTGCCCAGTTCAGTCCCTGTCATTGCCACGCTACTAAGGACTCCCTGAATACCGTGACTGACGTCA
GACATTGTCTGACTGAATACATCCTGTGGGTTTCTCATAGATGGACCCATAGAGAAAGCGCAGGGCCTCTCTACAGGCT
TCTCATCTCTTTCAGAACTGATGCAATGGAGCTCTTTGGTAGCGAGTTGAAGGAGTTCTCGGATTCACTTCCGTGGGAC
AATATCGACAATTGCGTGGAGATCATTAAATGTTTCATCAGAAATGACTCCATGAAAACCGCCAAAGAACTTTGTGCAA
TCATTGGACTTTGTACTCAATTAGCTATTGTCTCTGGAAGAGTCTTCAATGATAAGTATATCGACATACTACTTATGCT
GCGAAAGATTCTGAATGAGAACGACTATCTCACCCTCTTGGATCATATCCTCACT
SEQ ID NO: 49 is an example of a LAMP primer for the N gene of SARS-COV-2,
which may be referred to as F3.
GCTGCTGAGGCTTCTAAG
SEQ ID NO: 50 is an example of a LAMP primer for the N gene of SARS-COV-2,
which may be referred to as B3.
GCGTCAATATGCTTATTCAGC
SEQ ID NO: 51 is an example of a LAMP primer for the N gene of SARS-COV-2,
which may be referred to as FIP.
GCGGCCAATGTTTGTAATCAGTAGACGTGGTCCAGAACAA
SEQ ID NO: 52 is an example of a LAMP primer for the N gene of SARS-COV-2,
which may be referred to as BIP.
TCAGCGTTCTTCGGAATGTCGCTGTGTAGGTCAACCACG
SEQ ID NO: 53 is an example of a LAMP primer for the N gene of SARS-COV-2,
which may be referred to as LoopF.
CCTTGTCTGATTAGTTCCTGGT
SEQ ID NO: 54 is an example of a LAMP primer for the N gene of SARS-COV-2,
which may be referred to as LoopB.
TGGCATGGAAGTCACACC
SEQ ID NO: 55 is an example of a LAMP primer for the N gene of SARS-COV-2,
which may be referred to as N2-F3.
ACCAGGAACTAATCAGACAAG
SEQ ID NO: 56 is an example of a LAMP primer for the N gene of SARS-COV-2,
which may be referred to as N2-B3.
GACTTGATCTTTGAAATTTGGATCT
SEQ ID NO: 57 is an example of a LAMP primer for the N gene of SARS-COV-2,
which may be referred to as N2-FIP.
TTCCGAAGAACGCTGAAGCGGAACTGATTACAAACATTGGCC
SEQ ID NO: 58 is an example of a LAMP primer for the N gene of SARS-COV-2,
which may be referred to as N2-BIP.
CGCATTGGCATGGAAGTCACAATTTGATGGCACCTGTGTA
SEQ ID NO: 59 is an example of a LAMP primer for the N gene of SARS-COV-2,
which may be referred to as N2-LF.
GGGGGCAAATTGTGCAATTTG
SEQ ID NO: 60 is an example of a LAMP primer for the N gene of SARS-COV-2,
which may be referred to as N2-LB.
CTTCGGGAACGTGGTTGACC
SEQ ID NO: 61 is an example of a LAMP primer for the E gene of SARS-COV-2,
which may be referred to as E-F3.
TGAGTACGAACTTATGTACTCAT
SEQ ID NO: 62 is an example of a LAMP primer for the E gene of SARS-COV-2,
which may be referred to as E-B3.
TTCAGATTTTTAACACGAGAGT
SEQ ID NO: 63 is an example of a LAMP primer for the E gene of SARS-COV-2,
which may be referred to as E-FIP.
ACCACGAAAGCAAGAAAAAGAAGTTCGTTTCGGAAGAGACAG
SEQ ID NO: 64 is an example of a LAMP primer for the E gene of SARS-COV-2,
which may be referred to as E-BIP.
TTGCTAGTTACACTAGCCATCCTTAGGTTTTACAAGACTCACGT
SEQ ID NO: 65 is an example of a LAMP primer for the E gene of SARS-COV-2,
which may be referred to as E-LF.
CGCTATTAACTATTAACG
SEQ ID NO: 66 is an example of a LAMP primer for the E gene of SARS-COV-2,
which may be referred to as E-LB.
GCGCTTCGATTGTGTGCGT
SEQ ID NO: 67 is an example of a SDA primer and/or RPA primer for the E gene of
SARS-COV-2, which may be referred to as SDA_F1_in.
ACCGCATCGAATGCATGTGAGTCAAAATTTCGGAAGAGACAGGTAC
SEQ ID NO: 68 is an example of a SDA primer for the E gene of SARS-COV-2, which
may be referred to as SDA_bumpF1.
TGAGTACGAACTTATGTACTCAT
SEQ ID NO: 69 is an example of a SDA primer for the E gene of SARS-COV-2, which
may be referred to as SDA_bumpR2.
TAACAATATTGCAGCAGTACG
SEQ ID NO: 70 is an example of a SDA primer and/or RPA primer for the E gene of
SARS-COV-2, which may be referred to as SDA_R2_in.
GGATTCCGCTCCAGACTTGAGTCAAAACAATCGAAGCGCAGTAAG
SEQ ID NO: 71 is an example of a Sso7d DNA binding domain.
ATVKFKYKGEEKEVDISKIKKVWRVGKMISFTYDEGGGKTGRGAVSEKDAPKELLQMLEKQKK
SEQ ID NO: 72 is an example of an ET-SSB single stranded DNA binding domain.
MEEKVGNLKPNMESVNVTVRVLEASEARQIQTKNGVRTISEAIVGDETGRVKLTLWGKHAGSIKEGQVVKIENAWTTAF
KGQVQLNAGSKTKIAEASEDGFPESSQIPENTPTAPQQMRGGGRGERGGGRRYGRRGGRRQENEEGEEE
SEQ ID NO: 73 is an example of a linker that may be positioned between a variant
Cas12a and DNA binding domain.
SGGSSGGSSGSETPGTSESATPESSGGSSGGS
SEQ ID NO: 74 is an example of a wild type Lachnospiraceae bacterium Cas12a
(LbaCas12a).
MSKLEKFTNCYSLSKTLRFKAIPVGKTQENIDNKRLLVEDEKRAEDYKGVKKLLDRYYLSFINDVLHSIKLKNLNNYIS
LFRKKTRTEKENKELENLEINLRKEIAKAFKGNEGYKSLFKKDIIETILPEFLDDKDEIALVNSFNGFTTAFTGFFDNR
ENMFSEEAKSTSIAFRCINENLTRYISNMDIFEKVDAIFDKHEVQEIKEKILNSDYDVEDFFEGEFENFVLTQEGIDVY
NAIIGGFVTESGEKIKGLNEYINLYNQKTKQKLPKFKPLYKQVLSDRESLSFYGEGYTSDEEVLEVERNTLNKNSEIFS
SIKKLEKLFKNFDEYSSAGIFVKNGPAISTISKDIFGEWNVIRDKWNAEYDDIHLKKKAVVTEKYEDDRRKSFKKIGSF
SLEQLQEYADADLSVVEKLKEIIIQKVDEIYKVYGSSEKLFDADFVLEKSLKKNDAVVAIMKDLLDSVKSFENYIKAFF
GEGKETNRDESFYGDFVLAYDILLKVDHIYDAIRNYVTQKPYSKDKFKLYFQNPQEMGGWDKDKETDYRATILRYGSKY
YLAIMDKKYAKCLQKIDKDDVNGNYEKINYKLLPGPNKMLPKVFFSKKWMAYYNPSEDIQKIYKNGTFKKGDMENLNDC
HKLIDFFKDSISRYPKWSNAYDENESETEKYKDIAGFYREVEEQGYKVSFESASKKEVDKLVEEGKLYMFQIYNKDESD
KSHGTPNLHTMYFKLLFDENNHGQIRLSGGAELFMRRASLKKEELVVHPANSPIANKNPDNPKKTTTLSYDVYKDKRFS
EDQYELHIPIAINKCPKNIFKINTEVRVLLKHDDNPYVIGIDRGERNLLYIVVVDGKGNIVEQYSLNEIINNENGIRIK
TDYHSLLDKKEKERFEARQNWTSIENIKELKAGYISQVVHKICELVEKYDAVIALEDLNSGFKNSRVKVEKOVYQKFEK
MLIDKLNYMVDKKSNPCATGGALKGYQITNKFESFKSMSTQNGFIFYIPAWLTSKIDPSTGFVNLLKTKYTSIADSKKE
ISSFDRIMYVPEEDLFEFALDYKNFSRTDADYIKKWKLYSYGNRIRIFRNPKKNNVEDWEEVCLTSAYKELENKYGINY
QQGDIRALLCEQSDKAFYSSFMALMSLMLQMRNSITGRTDVDFLISPVKNSDGIFYDSRNYEAQENAILPKNADANGAY
NIARKVLWAIGQFKKAEDEKLDKVKIAISNKEWLEYAQTSVKH

### DETAILED DESCRIPTION

Tests employing isothermal amplification are simpler and low-cost compared to methods based on PCR because they do not require a thermal cycler or other expensive equipment. Isothermal amplification techniques continue to gain popularity following their widespread adoption for COVID detection in point-of-care settings, and there is a need for continued broadening of constant temperature test capabilities. Limitations of isothermal amplification techniques, such as undesired nonspecific amplifications which could lead to false-positive testing results and inability to differentiate single nucleotide polymorphisms, could be overcome by coupling the amplification with CRISPR-Cas enzymes in molecular diagnostics. Activation of the promiscuous trans nuclease activity of type V CRISPR/Cas, after recognition of specific target DNA by the CRISPR guide RNA, has been demonstrated to increase the detection sensitivity and specificity. However, amplification-CRISPR coupled detection methods suffer from potential cross-contamination issues when applied in two reaction steps in two vessels, i.e., two-pot detection. Accordingly, one-pot isothermal detection, in which components for isothermal amplification and detection by Type V CRISPR-Cas (e.g., Cas12b nucleases) are present in the same reaction mixture and incubated at the same temperature to allow for nucleic acid detection without further disruption to reaction once test sample is added is desirable. One-pot molecular detection coupling loop-mediated isothermal amplification (LAMP) and Cas12b has been reported. However, Cas12b guide RNAs are relatively long at 111 nt, adding expense and complexity to the development of diagnostic assays. Cas12a nucleases also have prominent trans nuclease activity but use much shorter guide RNA (˜41 nt), which makes them more economical than diagnostics based on Cas12b. However, very few thermostable Cas12a has been reported to be used in one-pot molecular diagnostics coupling isothermal amplifications, especially mid- to high-temperature reactions such as LAMP which is usually incubated at 55-65° C. Thus, it is desirable to develop one-pot isothermal detection methods coupling isothermal amplification with Cas12a.

Cas12a enzymes may bind a crRNA to form a ribonuclear protein complex (RNP), which may specifically recognize (by hybridization of the crRNA and) a target DNA (cis substrate). Target DNA specificity may be determined by its complementarity to the crRNA spacer sequence and the protospacer adjacent motif (PAM) required by Cas enzyme. When a target DNA is present, the RNP forms an R-loop structure and initiates cis substrate DNA cleavage by the RuvC nuclease domain. After PAM-distal cleavage product release, the post-cis cleavage product complex may remain stable and active to nonspecifically cleave DNA or RNA in trans (cleavage of trans substrate). Specific cis-substrate recognition is the basis for specificity, and nonspecific multi-turnover trans nuclease activity is the basis for signal generation and amplification in nucleic acid detection methods using Cas12a.

The present disclosure relates to solutions for conducting coupled isothermal amplification and Cas (e.g., Cas 12a) for nucleic acid detection in the same reaction mixture at the same temperature. For example, where loop-mediated isothermal amplification (LAMP) reactions occur at a relatively high temperature (e.g., around 55-65° C.), Cas enzymes (e.g., Cas 12a) may be sought that are stable and active at similar temperature range to be compatible for the coupled reaction. The present disclosure relates, in some embodiments, to Cas enzymes that are stable and active at elevated temperatures.

The highly programmable CRISPR/Cas system relies on guide RNAs of approximately 20 nt to recognize complementary nucleic acids with high specificity. The enzymes of the type V and VI families Cas12 and Cas 13 further activate non-specific trans nuclease activity upon specific target recognition by the guides, are thus widely used in molecular diagnostics. The sensitivity of CRISPR/Cas based nucleic detection itself is usually not high enough for direct detection. Thus, many CRISPR/Cas based molecular diagnostics have been coupled with isothermal nucleic acid amplification methods to leverage the advantages of both systems.

Available methods include two-pot nucleic acid detection comprising a first discrete step of isothermal amplification of target DNA followed by a second discrete step of Cas12 trans activity activation for signal amplification. However, due to the large quantity of DNA molecules generated during isothermal amplification, the opening of amplification vessels can easily result in carryover contamination in subsequent tests. Thus, one-pot diagnostics combining CRISPR/Cas systems and isothermal amplification is vital in large scale and repeated tests.

Molecular diagnostics coupling thermostable Cas 12b with RT-LAMP in one pot has been reported in the STOPCovid method (Joung J, et al. N Engl J Med. 2020 Oct. 8; 383 (15): 1492-1494. doi: 10.1056/NEJMc2026172). However, Cas12b guide RNAs are relatively long at 111 nt, adding expense and complexity to the development of diagnostic assays. Cas12a nucleases also have prominent trans nuclease activity but use much shorter guide RNA (˜41 nt), which could make them more economical than diagnostics based on Cas12b. For example, shorter guides may be advantageous (over long guides) as easier to make using standard oligonucleotide chemical synthesis techniques and/or more readily obtainable through commercial suppliers. However, existing Cas12a proteins lack helpful properties of Cas12b including thermostability and high trans nuclease activity. Determinants of Cas12b thermostability and high trans activity are not known or readily transferrable to Cas12a.

Molecular diagnostics coupling Cas12a with low temperature isothermal amplification in one pot (i.e. RPA at 37° C.) has been reported, but many diagnostics widely in use are high temperature isothermal amplification methods, such as LAMP (at 55-65° C.). Molecular diagnostics coupling Cas12a with a low temperature variation of LAMP in one pot (i.e. 52° C.) has been reported to bypass the requirement for thermostable Cas12a in high temperature one-pot nucleic acid detection. However, to balance low temperature variation of LAMP and Cas12a reactions for efficient one-pot detection, extensive reaction condition optimization, such as Mg²⁺ concentration and additives such as pyrophosphatase needs to be performed. Furthermore inner primers need to be phosphorothioated. These procedures and requirements are costly both in method development time and reagents procurement.

A single tube reaction of Cas12a coupled with high temperature LAMP (i.e. 62° C.) has been reported, however, to avoid heat deactivation of mesophilic Cas12a, the reagents for the amplification and Cas12a detection had to be physically separated, i.e. Cas12a reagents stay on the cap of the tube and separated from LAMP reaction which occurs at 62° C. on the bottom of the tube. Thus, robust and true one-pot Cas12a coupled molecular diagnostic method would be beneficial for many diagnostics that currently use isothermal amplification methods only. A thermostable and highly efficient Cas12a that could broadly enable one-pot nucleic acid detection coupling with wide range of isothermal amplification methods ranging from low (e.g., 25-37° C.) to high temperatures (e.g., 55-65° C.) is desirable to meet the requirements. Variants of Cas12a nucleases described herein are thermostable and highly active at a large temperature range. Thus, they may be used in one-pot nucleic acid detection reactions coupled with various isothermal amplification methods, i.e. in a single-reaction mixture at the same temperature.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and case of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., ≥90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10). Concentration percentages are w/v percentages unless otherwise indicated.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “catalytically active”, with respect to an enzyme, refers to any ability of the enzyme to detectibly convert substrate(s) to product(s). Catalytically active Cas12a may bind a gRNA to form an RNP, which recognizes, binds, and (optionally) cleaves DNA substrates that are complementary to the guide RNA (cis substrates) by the RuvC nuclease domain. After cis substrate binding and (optionally) cleavage, catalytically active Cas12a may further bind and cleave trans substrates. For clarity, cis recognition activity (capacity to specifically recognize and bind a DNA target sequence) and cis cleavage activity (capacity to cleave at such DNA target sequence), together, constitute cis activity. A catalytically active Cas12 has trans activity and cis recognition activity; optionally, it may further have cis cleavage activity. An example activity assay for Cas12a trans nuclease activity is described in Example 1. Conversion of substrate to product can be observed, for example, as a band on a gel, a substrate for a second enzyme or reaction, a fluorescent or colorimetric signal, and formation of precipitation. While an enzyme that displays any detectable activity in the assay of Example 1 may be regarded to be catalytically active, it may be desirable to select a higher threshold of activity for specific applications and embodiments.

In the context of the present disclosure, “contacting” refers to any act of bringing into contact one item (e.g., a molecule or group of molecules) with one or more other items (e.g., a second molecule or group of molecules, like or unlike the first molecule(s)). Contacting, for example, an enzyme and a substrate or a binding protein and its corresponding target, may include providing suitable conditions (e.g., concentration, pH, solvent, buffer, space (volume), temperature, time) and other parameters for the two materials to associate (e.g., for an enzyme to operatively interact with its substrate or a binding protein to bind its target). Contacting may be achieved by any method that brings two (or more) materials into operative association with one another including mixing (e.g., in solution), pouring, pipetting, flowing, injecting, vortexing, transferring, incubating, emulsifying, agitating, spraying, adhering, or coating one material with, in to, or on to another. Contacting includes, for example, adding, amalgamating, blending, combining, connecting, emulsifying, joining, mixing, precipitating, reacting, stirring, and/or touching one item with one or more other items.

In the context of the present disclosure, “contact” refers to any physical, chemical, electrical, magnetic or other association between two or more like or unlike materials (e.g., between two molecules).

In the context of the present disclosure, “container” refers to a human-made container. A container may comprise one or more walls (e.g., defining an interior volume) and optionally one or more openings. Containers comprising one or more openings may further comprise one or more closures (e.g., removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned (e.g., by the BLAST algorithm). An amino acid position in a functional or structural motif in one polymerase may correspond to a position within a functionally equivalent functional or structural motif in another polymerase.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag (e.g., a 6×His or 8×His tag), and expression tag, a polynucleotide binding protein, an enzyme, a conjugation tag (e.g., a SNAP® tag), and a peptide linker (e.g., a flexible linker, an inflexible linker, a cleavable linker). Unless otherwise disclosed, the protein of interest may be nearer to the N-terminal end or nearer to the C-terminal end than the second polypeptide to which it is joined. A fusion may comprise a non-naturally occurring combined polypeptide chain comprising two proteins or two protein domains joined directly to each other by a peptide bond or joined through a peptide linker. In some embodiments, a fusion may comprise a variant Cas12a domain covalently joined to a second polypeptide. In some embodiments, a variant Cas12a may include a fusion of a variant Cas12a domain to one or more activation domains (e.g., VPR). In some embodiments, a fusion may comprise a variant Cas12a linked to a DNA binding domain. Examples include DNA binding domain-Sso7d and ET-SSB. Other examples are listed in TABLE 1.

TABLE 1
DNA binding domains

| | Abbre- | |
|---|---|---|
| Name | viation | Accession |
| DNA-binding protein Tfx | BD-51 | gi\|499321160 |
| AbrB/MazE/MraZ-like | BD-52 | gi\|499321199 |
| “Winged helix” DNA-binding domain | BD-54 | gi\|499322061 |
| lambda repressor-like DNA-binding domains | BD-63 | gi\|499322443 |
| Resolvase-like | BD-67 | gi\|499322676 |
| “Winged helix” DNA-binding domain | BD-71 | gi\|499322676 |
| “Winged helix” DNA-binding domain | BD-74 | gi\|499322255 |
| “Winged helix” DNA-binding domain | BD-75 | gi\|499322388 |
| “Winged helix” DNA-binding domain | BD-81 | gi\|499322131 |
| “Winged helix” DNA-binding domain | BD-82 | gi\|499321342 |
| “Winged helix” DNA-binding domain | BD-85 | gi\|499321130 |
| “Winged helix” DNA-binding domain | BD-86 | gi\|499322705 |
| “Winged helix” DNA-binding domain | BD-88 | gi\|499320855 |
| “Winged helix” DNA-binding domain | BD-89 | gi\|499322250 |
| “Winged helix” DNA-binding domain | BD-91 | gi\|499321633 |
| “Winged helix” DNA-binding domain | BD-92 | gi\|490170077 |
| “Winged helix” DNA-binding domain | BD-94 | gi\|499320919 |
| “Winged helix” DNA-binding domain | BD-97 | gi\|499320853 |
| “Winged helix” DNA-binding domain | BD-98 | gi\|499321734 |
| “Winged helix” DNA-binding domain | BD-100 | gi\|499322439 |
| “Winged helix” DNA-binding domain | BD-102 | gi\|499322707 |
| HCP-like | BD-02 | gi\|351675391 |
| Helix-turn-helix domain, rpiR family | BD-03 | gi\|500479591 |
| Helix-turn-helix domain, rpiR family | BD-04 | gi\|15643984 |
| Bacterial regulatory proteins, lacI family | BD-08 | gi\|15643974 |
| Bacterial regulatory proteins, lacI family | BD-11 | gi\|500480095 |
| “Winged helix” DNA-binding domain | BD-14 | gi\|15644350 |
| “Winged helix” DNA-binding domain | BD-16 | gi\|24159093 |
| “Winged helix” DNA-binding domain | BD-18 | gi\|15643139 |
| “Winged helix” DNA-binding domain | BD-24 | gi\|15643159 |
| “Winged helix” DNA-binding domain | BD-30 | gi\|15643333 |
| “Winged helix” DNA-binding domain | BD-32 | gi\|15643055 |
| “Winged helix” DNA-binding domain | BD-37 | gi\|15643827 |
| “Winged helix” DNA-binding domain | BD-43 | gi\|15643699 |
| Homeodomain-like | BD-45 | gi\|15643788 |

In the context of the present disclosure, “≥75% identical”, with reference to amino acid or nucleic acid sequences, refers to and includes ≥75%, ≥80%, ≥85%, ≥90%, ≥92%, ≥94%, ≥95%, ≥96%, ≥98%, and ≥99% identity.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin:biotin, chitin:CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, isothermal DNA amplification approaches rely on the strand displacement activity of the DNA polymerase. The term “isothermal” as used herein means a constant temperature, as opposed to cycling between temperatures. Such isothermal amplification methods include strand displacement amplification (SDA) (see, e.g., Milla et al, Biotechniques 1998; 24:392-6), linear target isothermal multimerization and amplification (LIMA) (see, e.g., Hafner et al., Biotechniques 2001; 30:852-6), loop-mediated isothermal amplification (LAMP) (see, e.g., Notomi et al., E63 Nucleic Acids Res 2000; 28), nicking enzyme amplification reaction (NEAR) (see, e.g., US20090081670A1); recombinase polymerase amplification (RPA) (see, e.g., Piepenburg et al., PLOS Biol. 2006; 4 (7): c204); recombinase-assisted amplification (RAA) (see, e.g., Chen et al., Analyst 2020; 145:440-4); whole genome amplification, Multiple-strand Displacement Amplification (MDA) (e.g., extending DNA isolated from tissue (fresh, frozen, or preserved); see, e.g., Aviel-Ronen S, ct al. BMC Genomics. 2006 Dec. 12; 7:312), and HDA (helicase dependent amplification) (see, e.g., Vincent et al., EMBO J 2004; 5 (8): 795-800) and library prep, including whole genome amplification.

In some embodiments, the isothermal reaction is a LAMP reaction. LAMP reactions use several primers (generally, from four to six primers) that bind to locations on the target nucleic acid (“LAMP primers”). Guidance for selecting LAMP primers, including use of online software such as NEB LAMP primer design tool, PrimerExplorer, LAMP Designer Optigene, and Premier Biosoft, is well known in the art (see, for example, Parida et al., Rev. Med. Virol, 2008, 18:407-421 and Nagamine et al. Mol. Cell Probes 2002, 16, 223-229). Variations of LAMP reactions include reverse transcription loop-mediated isothermal amplification (RT-LAMP), multiplex loop-mediated amplification (M-LAMP). RT-LAMP reactions use reverse transcriptase activity combined with DNA polymerase activity. In some embodiments, a one-pot method comprising amplifying a polynucleotide of interest may include an RT-LAMP reaction to amplify RNA and enable detection of RNA sequences in a sample. For example, amplifying a polynucleotide of interest may comprise contacting a subject RNA and a reverse transcriptase to produce a DNA of interest and contacting the DNA of interest, a DNA polymerase, and desired or appropriate primers (e.g., in the presence of dNTPs) to produce a DNA amplification product. Similarly, a reverse transcriptase enzyme can be included in other isothermal amplification procedures, such as SDA and RPA, to enable one-pot assays that couple amplification and Cas12a in specific detection of RNA sequences in a sample.

In the context of the present disclosure, “one-pot reaction” refers to a reaction in which two or more reaction steps occur in a single reaction mixture and in a single reaction container (e.g., a tube, a well, a capillary, a surface). Sequential reaction steps in a one-pot reaction may begin and/or continue without changes to reaction conditions (e.g., without addition or removal of reagents, pH, volume, or washing, without opening a closed reaction container, without redistributing the contents of a closed reaction container) beyond those that arise or follow from the reactions themselves. For example, a one-pot reaction may include a reaction in which a nuclease (e.g., a thermostable Cas12a nuclease) is contacted with a polymerase (e.g., an isothermal polymerase) in a single reaction container (e.g., to form a single reaction mixture) and both cleavage (e.g, trans nuclease cleavage) and synthesis (e.g., polymerase-mediated DNA synthesis) reactions proceed in tandem in the same mixture (e.g., without an intervening change in temperature and without a purification step or with a temperature change but without a purification step). For clarity, one-pot reactions include reactions in which microenvironments may exist (e.g., in and/or on the surface of the reaction mixture and/or reaction container) in an otherwise contiguous fluid system (e.g., a single reaction mixture). For further clarity, a one-pot reaction may include, after reaction components are combined in a single mixture in a single reaction container at a first temperature (e.g., a temperature at which the included enzymes are catalytic activity inactive or at most have nominal activity), changing (e.g., increasing) the temperature to a single temperature at which included enzymes are catalytically active (e.g., optimally or near optimally active), thereby commencing the one-pot reaction. In such cases, the temperature change necessarily includes all reaction components since all reaction components are present in the single mixture. For example, all components may be combined in the single mixture in the container at 0° C. (e.g., in a thermocycler or on ice; conditions under which the included enzymes are catalytic activity inactive or at most have nominal activity) and then the temperature may be increased to 55° C. to facilitate both polymerase and nuclease activities. As set forth herein, a one-pot reaction would not include reactions having reaction components (e.g., a polymerase and a nuclease) in a single container, but spatially separated from one another (e.g., a polymerase in the bottom of the tube and the nuclease in the cap), so that one reaction can proceed (e.g., amplification in the bottom of the tube) in a reaction volume that excludes or substantially excludes one or more components needed for the second reaction (e.g., nuclease for cleavage). Steps of one-pot reactions may benefit from or require compatibility of reaction conditions including, for example, buffers, substrates, products, enzymes, and/or other reaction mixture components. For example, a one-pot reaction comprising amplification of a template (e.g., by LAMP) and nucleolytic cleavage (e.g., by Cas12a) of an amplification product and/or a detector probe may employ enzymes for each reaction that are catalytically active at a desired reaction temperature (e.g., 55° C.), at a desired concentration of one or more dNTPs, and/or in the presence of a desired salt concentration.

In some embodiments, the presence of reagents for amplification and detection in one reaction mixture (one pot), as compared to two containers, may reduce or eliminate the false-positive problem associated with amplicon contamination due to accidental dispersal of amplicons when the amplification container is opened to initiate the next reaction (or carryover contamination). A one-pot reaction comprising amplification (e.g., LAMP) and nucleolytic cleavage (Cas12a) may include homogeneous real time detection while, by contrast, separated reaction components (e.g., in separate tubes or in one tube where components are spatially separated) constitute heterogenous detection. One-pot reactions may be advantageously easier to perform, more sensitive, and/or more specific than heterogenous detection reactions.

In the context of the present disclosure, “single temperature one-pot reaction” refers to one-pot reaction that occurs at a single temperature. For clarity, a reaction may be regarded as being performed at a single temperature even if (notwithstanding the exercise of reasonable care), a reaction temperature undergoes minor fluctuation, for example, due to variations in the performance of equipment used. A single temperature one-pot reaction may also be referred to as an isothermal one-pot reaction.

With reference to an amino acid, “position” refers to the place such amino acid occupies in the primary sequence of a peptide or polypeptide numbered from its amino terminus (N-terminus) to its carboxy terminus (C-terminus).

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid, a polar amino acid (e.g., N, Q, S, T, and Y) may substitute for another polar amino acid (e.g., C, D, E, H, K, N, P, Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid).

In the context of the present disclosure, “thermostable” refers to a property of an enzyme wherein such enzyme retains a desired fraction of a desired catalytic activity (e.g., trans nuclease activity) at or upon or after exposure to an elevated temperature. A thermostable enzyme may retain, for example, ≥5%, ≥10%, ≥15%, ≥20%, ≥25%, ≥30%, ≥35%, ≥40%, ≥45%, ≥50%, ≥55%, ≥60%, ≥65%, ≥70%, ≥75%, ≥80%, ≥85%, ≥90% of its catalytic activity at or upon or after exposure to a temperature of ≥45° C., ≥50° C., ≥55° C., ≥60° C., ≥65° C. for ≥2 minutes, ≥3 minutes, ≥4 minutes, ≥5 minutes, ≥6 minutes, ≥8 minutes, ≥10 minutes, ≥20 minutes, ≥30 minutes when compared to its activity in a control reaction without exposure to the elevated temperature. For example, a thermostable variant Cas12a exposed to a temperature of ≥45° C., ≥50° C., ≥55° C., ≥60° C., ≥65° C. for ≥2 minutes, ≥3 minutes, ≥4 minutes, ≥5 minutes, ≥6 minutes, ≥8 minutes, ≥10 minutes, ≥20 minutes, ≥30 minutes may retain ≥10%, ≥25%, ≥50%, ≥75%, ≥80%, ≥85%, ≥90% of its cis recognition and/or cis cleavage activity and/or retain its trans cleavage activity as compared to the same enzyme assayed under the same conditions without the exposure to the elevated temperature. Examples of thermostable variant Cas12a include enzymes having an amino acid sequence that is at least 80% identical (e.g., ≥85%, ≥90%, ≥92%, ≥94%, ≥95%, ≥97%, ≥98% identical) to an amino acid sequence selected from: SEQ ID NOS: 1-16. Examples of desirable thermostable variant Cas12a enzyme performance are shown in TABLE 2, wherein retained activity is calculated as a percentage of the enzyme's peak activity (its the highest observed activity in the same reaction mixture under any time/temperature conditions). Thermostable Cas12a enzymes may be desirable versus non-thermostable enzymes for one or more reasons, including allowing and/or performing better in one-pot and/or isothermal detection workflows. For example, it may be beneficial to do things in one step because it saves time and simplifies the workflow of the reaction; if not thermostable the user has to both conduct a second step increasing time and complexity by adding a second temperature. A second step also requires opening the reaction vessel which introduces risk of workspace or laboratory contamination and may make some workflows (e.g., automated workflows) impractical or impossible. In devices or instruments doing these reactions, 2 temperature spaces are required, and reaction mixture must be moved around and/or a second reaction mixture be added, resulting in and more complex and expensive design.

TABLE 2

| Example | Test Reaction | | Retained |
|---|---|---|---|
| Enzyme | Time (min) | Temperature | Activity |
| A | 5 | ≥50° C. | ≥85% |
| B | 10 | ≥50° C. | ≥85% |
| C | 15 | ≥50° C. | ≥85% |
| D | 30 | ≥50° C. | ≥85% |
| E | 60 | ≥50° C. | ≥85% |
| F | 5 | ≥55° C. | ≥85% |
| G | 10 | ≥55° C. | ≥85% |
| H | 15 | ≥55° C. | ≥85% |
| I | 30 | ≥55° C. | ≥85% |
| J | 60 | ≥55° C. | ≥85% |
| K | 5 | ≥60° C. | ≥75% |
| L | 10 | ≥60° C. | ≥75% |
| M | 15 | ≥60° C. | ≥75% |
| N | 30 | ≥60° C. | ≥75% |
| O | 60 | ≥60° C. | ≥75% |
| P | 5 | ≥65° C. | ≥60% |
| Q | 10 | ≥65° C. | ≥60% |
| R | 15 | ≥65° C. | ≥60% |
| S | 30 | ≥65° C. | ≥60% |
| T | 60 | ≥65° C. | ≥60% |
| U | 5 | ≥55° C. | ≥75% |
| V | 10 | ≥55° C. | ≥60% |
| W | 15 | ≥55° C. | ≥50% |

In the context of the present disclosure, “variant Cas12a” refers to a non-naturally occurring nuclease having both cis binding and trans nuclease activity at a range of temperatures, for example, 1° C.-65° C., 10° C.-60° C., 15° C.-55° C., 50° C.-60° C., 55° C.-65° C., and/or 50° C.-70° C. A variant Cas12a may have a bilobed structure comprising a REC lobe and a Nuc lobe. A variant Cas12a may comprise, in its REC lobe, a REC1 domain and a REC2 domain and may have, in its Nuc lobe, a Ruv-C like nuclease domain (e.g., comprising RuvC I-III), a PAM-interacting domain, a WED domain, and a bridge helix. For example, a variant Cas12a may comprise, in an N-terminal to C-terminal direction, a REC lobe and a NUC lobe. 10 A variant Cas12a may comprise, in an N-terminal to C-terminal direction, a WED-I domain, a REC1 domain, a REC2 domain, a WED-II domain, a PAM interacting domain, a WED-III domain, a RevC-I domain, a bridge helix domain, a RevC-II domain, a NUC domain, and a RevC-III domain, wherein the WED-I, REC1, and REC2 domains may form a REC lobe and wherein the WED-II domain, the PAM interacting domain, the WED-III domain, the RevC-I domain, the bridge helix domain, the RevC-II domain, the NUC domain, and the RevC-III domain may form a NUC lobe. A variant Cas12a may comprise a DNA binding domain (e.g., as provided in TABLE 1). According to some embodiments, a variant Cas12a may include an arginine-rich region and/or a zinc finger. A catalytically active variant Cas12a binds a crRNA to form a ribonucleoprotein (RNP) to recognize, bind, and cleave a DNA substrate that is complementary to the crRNA spacer and meets the PAM requirement of the Cas12a. After cis substrate recognition, Cas12a variants may further show non-specific nuclease activity on one or more trans substrates, which can be dsDNA, ssDNA, or RNA. The variant Cas12a may be generated by ancestral sequence reconstruction, or by introducing one or more changes to the amino acids of the wildtype YmeCas12a, or additionally by fusing a DNA binding domain to the N or C terminal ends of the protein.

A variant Cas12a may comprise one or more amino acids in addition to a wild type Cas12a. For example, a variant Cas12a may comprise (e.g., at its amino terminal end or carboxy terminal end) 1-25 amino acids more than a reference (e.g., wild type) sequence. Such additional amino acids may enable, facilitate and/or enhance translation, expression, cellular sorting, inactivation (e.g., by including a protease recognition and/or cleavage site), and/or purification. Such additional amino acids may constitute a linker, for example, to a support (e.g., a magnetic bead) or another protein.

A variant Cas12a as disclosed herein may differ from one or more wildtype Cas12a and/or one or more other Cas12a molecules. For example, a variant Cas12a may have more and/or different electrostatic interactions, hydrophilic contacts, hydrogen bonds, and/or metal binding sites. In some embodiments, a variant Cas12a may differ from one or more wildtype Cas12a and/or one or more other Cas12a molecules in the number (e.g., the variant having fewer) and/or length (e.g., the variant having shorter) loops than a reference wildtype or other compared Cas12a.

A variant Cas12a may have an amino acid sequence sharing any desired degree of sequence identity with a wild type Cas12a up to (but excluding) 100% identity. For example, a variant Cas12a may have an amino sequence having at least 75%, at least 80%, at least 85%, at least 90%, at least 92%, at least 94%, at least 95%, at least 98%, or at least 99% identity to SEQ ID NO:17, optionally including a substitution (e.g., a non-conservative substitution) at one or more of its positions that correspond to position 1019 and/or 1155 of SEQ ID NO: 17 (or position 1006 and/or 1142 of SEQ ID NO:7). Example substitutions include G1019K and/or 11155R of SEQ ID NO:17, or G1006K and/or 11142R of SEQ ID NO:7. A variant Cas12a may also have a domain substitution, for example, a helix-loop-helix region corresponding to amino acids 82-88 of LbaCas12a (RKKTRTE; SEQ ID NO:74) instead of amino acids 98-117 of YmeCas12a (SEQ ID NO:17). Examples of variant Cas12a include catalytically active proteins having an amino acid sequence ≥75%, ≥80%, ≥85%, ≥90%, ≥92%, ≥94%, ≥95%, ≥96%, ≥98%, or ≥99% identical to one or more of SEQ ID NOS: 1-16.

As used herein, the term “crRNA” or “guide”, “guide RNA” or “single guide RNA” refers to a polynucleotide comprising any polynucleotide sequence having sufficient complementarity with a target nucleic acid sequence to hybridize with the target nucleic acid sequence and to direct sequence-specific binding of a DNA-targeting complex comprising the crRNA and a CRISPR effector protein to the target nucleic acid sequence. In general, a crRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence. As used herein the term “capable of forming a complex with the CRISPR effector protein” refers to the crRNA having a structure that allows specific binding by the CRISPR effector protein to the crRNA such that a complex is formed that is capable of binding to a target DNA in a sequence specific manner and that can exert a function on said target DNA. Structural components of the crRNA may include direct repeats and a guide sequence (or spacer). The sequence specific binding to the target DNA is mediated by a part of the crRNA, the “guide sequence”, or “spacer”, being complementary to the target DNA.

In embodiments of the invention the term guide RNA, i.e. RNA capable of guiding Cas to a target locus, is used as in foregoing cited documents such as WO 2014/093622 (PCT/US2013/074667). As used herein the term “wherein the guide sequence is capable of hybridizing” refers to a subsection of the crRNA having sufficient complementarity to the target sequence to hybridize thereto and to mediate binding of a CRISPR complex to the target DNA. In general, a guide sequence is any polynucleotide sequence having sufficient complementarity with a target polynucleotide sequence to hybridize with the target sequence and direct sequence-specific binding of a CRISPR complex to the target sequence.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Enzymes

The present disclosure relates, in some embodiments, to CRISPR-associated nucleases (e.g., variant Cas12a enzymes) having one or more desirable properties including, for example, thermostable cis recognition activity and/or trans catalytic activity. For example, a variant Cas12a may have both thermostable cis recognition activity and thermostable trans activity. A CRISPR-associated nuclease additionally or alternatively may have one or more desirable properties including, for example, a variant Cas12a may exhibit higher trans nuclease activity compared to wild type Cas12a Yme when compared under the same conditions. A variant Cas12a may exhibit higher trans nuclease activity (compared to wild type Cas12a Yme when compared under the same conditions) in the presence of inhibitors, including, for example, dNTPs (e.g., at concentrations ≤0.2 mM, ≤0.4 mM, ≤0.6 mM, ≤0.8 mM, ≤1 mM, ≤1.2 mM, ≤1.4 mM, ≤2 mM)) and NaCl (e.g., at concentrations ≤50 mM, ≤100 mM, ≤150 mM, ≤200 mM)). A variant Cas12a may exhibit less target-dependent activity variation than is observed in many Cas nucleases. For example, given the same set of guides and compatible targets, a variant Cas12a may show prominent trans nuclease activity and enable one-pot nucleic acid detection when coupled with isothermal amplification with 80% of the guides, whereas a wild type Cas12a may be successful with 10% of the guides. A variant Cas12a may exhibit less sequence and/or size bias for the trans substrate. A variant Cas12a may exhibit high trans nuclease activity at a larger temperature range (e.g., ≥33° C., ≥35° C., ≥37° C., ≥39° C., ≥40° C., ≥42° C., ≥44° C., ≥46° C. or any range between such temperatures). A variant Cas12a may exhibit broader PAM compatibility, such as “YYN” wherein Y represents T or C, and N represents A, G, T, or C, whereas a wild type Cas12a may be compatible with “TTN” PAM.

In some embodiments, a CRISPR-associated nuclease (e.g., a variant Cas12a) may be encoded by a nucleic acid sequence that, when transcribed, translated, and/or processed, results in an amino acid sequence having at least 80%, at least 85%, at least 90%, at least 93%, at least 96%, at least 97%, at least 98% or at least 99% identity to any of SEQ ID NO: 1-16. A nucleic acid encoding a CRISPR-associated nuclease may be included in an expression cassette, expression vector, or other expressible form suitable for in vitro or in vivo expression (e.g., in E. coli or other bacteria or P. pastoris or other yeast). A nucleic acid encoding a CRISPR-associated nuclease may be modified or optimized (e.g., codon optimized) for expression in a desired organism or cell-free expression system.

A CRISPR-associated nuclease (e.g., a variant Cas12a), in some embodiments, may be catalytically active on trans substrates and optionally cis substrates (e.g., in liquid aqueous media) at temperatures ≥1° C., ≤20° C., ≤25° C., ≤30° C., ≤35° C., ≤40° C., ≤45° C., ≤50° C., ≤55° C., ≤60° C., and/or ≤65° C. In some embodiments, a variant Cas12a may be catalytically active on trans substrates and optionally cis substrates at temperatures ≥35° C., ≤40° C., ≤45° C., ≤50° C., ≤55° C., ≤60° C., and/or ≤65° C. In some embodiments, a variant Cas12a may be catalytically active on trans substrates and optionally cis substrates at temperatures ≥45° C., ≤50° C., ≤55° C., ≤60° C., and/or ≤65° C.

The present disclosure relates, in some embodiments, to an immobilized enzyme comprising a support and an enzyme immobilized thereto. For example, an immobilized enzyme (e.g., a variant Cas12a or a polymerase) may comprise the enzyme, a glycine-serine linker attached to the enzyme by a peptide bond, a protein tag (e.g., a SNAP-tag) attached to the linker by a peptide bond, O⁶-benzyleguanine bound to the protein tag (e.g., SNAP-tag®); and magnetic beads having a surface modification comprising the O⁶-benzyleguanine. In some embodiments, a support of an immobilized enzyme may comprise a magnetic bead. A magnetic bead may comprise, for example, one or more surface modifications. Surface modifications may include, for example, 06-benzyleguanine and/or PEG750. In some embodiments, an immobilized enzyme may comprise a ligand (e.g., 06-benzyleguanine) and a receptor or tag (e.g., a SNAP-tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., a Cas12 or a polymerase), optionally, a first linker (e.g., a peptide linker) attached to the enzyme, a polypeptide tag (e.g., a SNAP-tag®) attached to the first linker, if present, or the enzyme, a ligand corresponding to the polypeptide tag (e.g., O⁶-benzyleguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated as: ENZYME-[LINKER-|TAG-LIGAND-[LINKER-|SUPPORT, wherein dashes represent bonds (covalent or non-covalent) and brackets represent optional elements.

In some embodiments, CRISPR-associated nucleases (e.g., a variant Cas12a) and compositions comprising one or more CRISPR-associated nucleases (e.g., a variant Cas12a) may have any desirable form including, for example, a liquid, a gel, a film, a powder, a cake, and/or any dried or lyophilized form. A CRISPR-associated nuclease composition may comprise a CRISPR-associated nuclease (e.g., a variant Cas12a) and a support or matrix, for example, a film, gel, fabric, or bead comprising, for example, a magnetic material, agarose, polystyrene, polyacrylamide, and/or chitin. A CRISPR-associated nuclease and compositions comprising a CRISPR-associated nuclease may be thermostable. For example, a CRISPR-associated nuclease or a CRISPR-associated nuclease composition may display CRISPR-associated nuclease activity at 25° C.-65° C.

### Compositions

The present disclosure further relates to compositions comprising a means for selectively cleaving a DNA strand. Means for selectively cleaving a DNA strand may include, for example, a CRISPR-associated nucleases (e.g., a variant Cas12a). A composition may comprise, for example, a means for selectively cleaving a DNA strand, wherein the selective cleavage comprises cleaving ≥90% (or optionally ≥92%, ≥94%, ≥95%, ≥96%, ≥98%, or ≥99% (each, a molar percent)) of the DNA strands and one or more additional components. Means for cleaving the DNA strand include all of the enzymes having the physical and chemical properties disclosed herein (e.g., sequences, motifs, bonds, binding properties, and other structures and features). Example means for cleaving a DNA strand include CRISPR-associated nucleases (e.g., variant Cas12a enzymes).

A CRISPR-associated nuclease may comprise, for example, a CRISPR-associated nuclease variant (e.g., having an amino acid sequence at least 75% identical to one or more of SEQ ID NO:1-16). A CRISPR-associated nuclease composition may be cell-free and/or free of one or more other catalytic activities (apart from the cis and trans activities of Cas itself).

For example, a CRISPR-associated nuclease may be free of nucleases that cleave dsRNA, free of nucleases that cleave ssDNA, free of nucleases that cleave ssRNA (e.g., free of DNase I, RNase), free of DNA polymerase activity, free of RNA polymerase activity, and/or free of protease activity, in each case, under desired one-pot conditions (e.g., conditions of time, temperature, pH, salinity, model substrate and/or others), for example, conditions intended to replicate conditions of a specific use of the CRISPR-associated nuclease composition or intended to represent conditions for a range of uses. A composition having a CRISPR-associated nuclease (e.g., a variant Cas12a) optionally may be free of any other enzyme or all other enzymes, according to some embodiments. For example, a composition comprising a CRISPR-associated nuclease may have cis recognition activity, but lack cis nuclease activity. A composition comprising a CRISPR-associated nuclease may lack a DNA polymerase (e.g., any specific DNA polymerase or all DNA polymerases), for example, where it may be desirable to avoid synthesizing unwanted DNA that could be digested by the CRISPR-associated nuclease. A composition comprising a CRISPR-associated nuclease may lack an RNA polymerase (e.g., any specific RNA polymerase or all RNA polymerases), for example, where it may be desirable to avoid synthesizing unwanted RNA that could form RNA: DNA duplexes. Similarly, a composition may lack one or more polymerase substrates (e.g., dNTPs, NTPs) to minimize or prevent undesired synthesis activity. In some embodiments, a composition comprising a CRISPR-associated nuclease may lack a protease (e.g., any specific protease or all proteases), for example, where it is desirable to avoid inadvertent or unintended cleavage of the CRISPR-associated nuclease and/or one or more other proteins present in the composition.

According to some embodiments, a CRISPR-associated nuclease composition may comprise a CRISPR-associated nuclease (e.g., a variant Cas12a) and, optionally, any of (including one or more of) a guide, a buffering agent (e.g., a storage buffer, a reaction buffer), an excipient, a salt (e.g., NaCl, MgCl₂, CaCl₂)), a protein (e.g., albumin, topoisomerase, polymerase), a stabilizer, a detergent (for example, ionic, non-ionic, and/or zwitterionic detergents (e.g., octoxinol, polysorbate 20), a polynucleotide (e.g., a guide, an amplification substrate, a cis substrate, a trans substrate), a cell (e.g., intact, digested, or any cell-free extract), a biological fluid or secretion (e.g., mucus, pus), an aptamer, a pH indicator (e.g., azolitimin, bromocresol purple, bromothymol blue, methylene blue, cresol red, neutral red, naphtholphthalein, phenol red), a crowding agent, a sugar (e.g., a mono, di, tri, tetra, or higher saccharide), a starch, cellulose, a glass-forming agent (e.g., glycerol, raffinose, stachyose, or trehalose for lyophilization), a lipid, an oil, aqueous media, a support (e.g., a bead) and/or (non-naturally occurring) combinations thereof. Combinations may include for example, two or more of the listed components (e.g., a salt and a buffer) or a plurality of species of a single listed component (e.g., two different salts or two different sugars). In some embodiments, a composition may be free of any of the above-listed components (e.g., a glycerol free composition comprising a variant Cas12a and/or a polymerase). According to some embodiments, CRISPR-associated nuclease compositions may comprise (a) a CRISPR-associated nuclease (e.g., a variant Cas12a), (b) a guide comprising a sequence complementary to a target sequence, (c) a polynucleotide (e.g., a library, a biological material, or other material comprising a plurality of polynucleotides) comprising or potentially comprising the target sequence, and (d) optionally, a reporter polynucleotide.

A composition, in some embodiments, may comprise one or more amplification substrates, cis recognition substrates, and/or trans nuclease substrates. For example, an amplification substrate may comprise RNA or DNA that is operable to be amplified. A cis recognition substrate and/or a cis nuclease substrate may comprise, for example, a DNA that is complementary to the spacer sequence of a guide RNA. A trans nuclease substrate may comprise a reporter polynucleotide (e.g., DNA or RNA) having, for example, a cleavable linker strand (e.g., 5-25 nucleotides), a fluorophore on or towards (e.g., 1-6 nucleotides from) one end of the linker and a quencher on or towards (e.g., 1-6 nucleotides from) the other end of the linker, wherein the fluorophore and the quencher are positioned in sufficiently close spatial proximity for the quencher to quench fluorescence of the fluorophore. Upon cleavage of the linker DNA, the fluorophore and the quencher are free to separate, whereupon the fluorophore is freed to fluoresce. In some embodiments, a variant Cas12a trans substrate may comprise a chemical modification. For example, a trans substrate may comprise a fluorophore (e.g., for fluorescence detection) and/or a biotin group (e.g., for lateral flow detection).

A composition may comprise, in some embodiments, one or more NTPs and/or one or more dNTPs, for example, as an energy source (e.g., ATP) and/or a reaction substrate (e.g., for amplification and/or transcription). A composition, in some embodiments, may comprise one or more modified nucleotides (e.g., base modified nucleotides, sugar modified nucleotides, and/or labeled nucleotides) and/or one or more modified nucleotide linkages (e.g., thiol linkages, azido linkages).

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine, N1-methyl-pseudouridine, and 2-aminoadenine.

According to some embodiments, a composition may comprise a CRISPR-associated nuclease (e.g., a thermostable variant Cas12a) and a DNA polymerase (e.g., a thermostable DNA polymerase). Example polymerases include Bst DNA polymerase, Bst 2.0® DNA polymerase, Bst 3.0@ DNA polymerase, DNA polymerases disclosed in U.S. Pat. No. 63,610,498 filed Dec. 15, 2023 incorporated herein by reference (e.g., BD009-SDpol-1), Bsu DNA polymerase, phi29 DNA polymerase, phi29-XT DNA polymerase, and variants of the foregoing polymerases. According to some embodiments, a composition may comprise a CRISPR-associated nuclease (e.g., a thermostable variant Cas12a) and a reverse transcriptase (e.g., thermostable reverse transcriptase). Example reverse transcriptases include WarmStart® RTx reverse transcriptase, Induro® reverse transcriptase, AMV reverse transcriptase, ProtoScript® II reverse transcriptase, Luna® WarmStart® reverse transcriptase, template switching reverse transcriptase, M-MuLV reverse transcriptase, and variants thereof.

A composition may comprise, in some embodiments, a glycosylase, for example, to reduce or prevent carryover. Example glycosylases include uracil DNA glycosylase (UDG)), Antarctic Thermolabile UDG, WarmStart® Afu UDG, and Afu UDG.

According to some embodiments, a composition may comprise a nicking enzyme (e.g., a thermostable single-strand DNA nickase), for example, to form nicked DNA suitable for strand displacement amplification (SDA). Example DNA nicking enzymes include Nt.BstNBI, WarmStart® Nt.BstNBI, Nb.BbvCI, thermostable Cas9 nickase, Argonaute, and variants thereof.

A composition may comprise, in some embodiments, a ligase (e.g., a DNA ligase, an RNA ligase), for example, to form a joined (including circularized) polynucleotide, which polynucleotide may be amplified and/or transcribed. Example ligases include 9° N™ DNA ligase, Taq DNA ligase, Hi-T4 DNA ligase, T7 DNA ligase, and variants thereof. A composition (e.g., a composition comprising a ligase) may comprise one or more oligonucleotides, each having a sequence complementary to a target oligonucleotide (e.g., to facilitate ligation). A ligase may be thermostable, for example, for use in high temperature one-pot reactions.

According to some embodiments, a composition may comprise a dye, for example, a DNA intercalating dye. A dye may contact a molecule of interest (e.g., DNA) in a manner that allows visualization (or other detection) of the molecule of interest. Example, dyes include SYTO-9® double-stranded DNA binding dye, SYBR Green, SYBR gold, PicoGreen™, and TOTO-1.

According to some embodiments, a composition may comprise a helicase (e.g., a DNA helicase, an RNA helicase), for example, to separate the dsDNA strand at isothermal condition for helicase-dependent amplification (HDA). Example helicases include Tte UvrD helicase, E. coli UvrD helicase, T7 gene 4 protein and variants thereof. A helicase may be thermostable, for example, for use in one-pot reactions. A composition (e.g., a composition comprising a helicase) may include a helicase associating protein or enzyme, such as MutL.

A composition may comprise, in some embodiments, a DNA binding protein, for example, to stabilize ssDNA during isothermal amplification, such as in recombinase polymerase amplification (RPA). Example DNA binding proteins include T4 gene 32 protein, RB49 gene 32 protein, E. coli SSB, T7 gene 2.5 SSB, and variants thereof. A DNA binding protein may be thermostable, for example, for use in high temperature one-pot reactions.

According to some embodiments, a composition may comprise a recombinase, for example, to enable isothermal amplification in the recombinase polymerase amplification (RPA). Example recombinases include T4 UvsX and T4 UvsY. A recombinase may be thermostable, for example, for use in high temperature one-pot reactions.

A composition may comprise, in some embodiments, a crowding agent, for example, to promote desired contact between macromolecules of interest (e.g., enzymes and substrates). Example crowding agents include polyethylene glycol (such as PEG 200, PEG 5000, PEG 8000, PEG 20000, PEG35000), dextran T-70, Ficoll 70, sucrose, glucose, bovine plasma albumin, and Carbowax 20.

According to some embodiments, a composition may comprise an additive, for example, to stabilize and/or facilitate reactivity of substrates, enzymes, reactants, in storage compositions, kits, and/or reactions. Example additives may include taurine, guanidine dihydrochloride, urea, imidazole, trichloroacetic acid, amino acids, L-arginine ethyl ester dihydrochloride, L-arginamide dihydrochloride, 6-aminohexanoic acid, gly-gly, gly-gly-gly, tryptone, betaine, trehalose, xylitol, sorbitol, sucrose, hydroxy ectoine, trimethylamine N-oxide, methyl-a-D-glucopyranoside, tricthylene glycol, spermine, spermidine, 5-aminovaleric acid, adipic acid, ethylenediamine, N-methylurea, N-ethylurea, N-methylformadie, hypotaurine, TCEP hydrochloride, GSH, benzamidine hydrochloride, ethylenediaminetetraacetic acid, magnesium chloride, cadmium chloride, Tween 20, non detergent sulfobetaine 195, non detergent sulfobetaine 201, non detergent sulfobetaine 211, non detergent sulfobetaine 221, non detergent sulfobetaine 256, acetamide, oxalic acid, sodium malonate, succinic acid, tacsimate, tetraethylammonium bromide, cholin acetate, 1-ethyl-3-methylimidazolium acetate, 1-butyl-3-methylimidazolium chloride, ethylammonium nitrate, ammonium sulfate, ammonium chloride, magnesium sulfate, potassium thiocyanate, gadolinium (III) chloride, cesium chloride, 4-aminobutyric acid, lithium nitrate, malic acid, lithium citrate tribasic, ammonium acetate, sodium benzenesulfonate, sodium p-toluenesulfonate, sodium chloride, potassium chloride, sodium phosphate, sodium sulfate, lithium chloride, sodium bromide, glycerol, ethylene glycol, ethylene glycol 200, ethylene glycol 400, ethylene glycol 600, ethylene glycol 3350, ethylene glycol 8000, ethylene glycol 20000, polyethylene glycol monomethyl ether 550, polyethylene glycol monomethyl ether 750, formamide, 1,2-propanediol, polyethylene glycol monomethyl ether 1900, polyvinylpyrrolidone K15, (2-hydroxypropyl)-β-cyclodextrin, a-cyclodextrin, β-cyclodextrin, methyl-β-cyclodextrin, MES monohydrate, sodium acetate, sodium citrate, AMPD, and Carbowax 20.

In some embodiments, a composition may include a ribonucleoprotein (RNP). Example RNPs include a complex formed by a Cas12a (e.g., a variant Cas12a) and at least one guide RNA operable with such Cas12a. For example, a single a Cas12a (e.g., a variant Cas12a) may be associated with a plurality of guides (e.g., 2 or more, 3 or more, 4 or more).

Compositions may include any of the foregoing materials at any desired and/or effective concentration. Enzymes, for example, may be operative at nanomolar and/or micromolar concentrations, but may also be adjusted according to the specific activity of the enzyme. Non-enzymatic proteins may be included in similar amounts. Other components may be present, for example, in nanomolar, micromolar, or millimolar concentrations.

According to some embodiments, a composition may be glycerol free, animal free, and/or endotoxin free. In this context, “free” refers to having a level of the subject material that is below a definable threshold (e.g., a detection threshold, a regulatory threshold) and/or a definable effect on another component of the composition or an intended reaction or reaction product.

### Kits

The present disclosure further relates to kits. For example, a kit may include a means for selectively cleaving a DNA strand. In some embodiments, a kit may include a CRISPR-associated nuclease (e.g., a variant Cas12a) and/or a polymerase. For example, a kit may include a thermostable CRISPR-associated nuclease (e.g., a variant Cas12a) having at least 75% identity to any of SEQ ID NO: 1-16, a polymerase (e.g., a Bst DNA polymerase, Bst 2.0® DNA polymerase, Bst 3.0® DNA polymerase, DNA polymerases disclosed in U.S. Pat. No. 63,610,498 filed Dec. 15, 2023 (incorporated herein by reference), Bsu DNA polymerase, phi29 DNA polymerase, phi29-XT DNA polymerase, and variants thereof), a reverse transcriptase (e.g., WarmStart® RTx reverse transcriptase, Induro® reverse transcriptase, AMV reverse transcriptase, ProtoScript® II reverse transcriptase, Luna® WarmStart® reverse transcriptase, template switching reverse transcriptase, M-MuLV reverse transcriptase, and variants thereof), dNTPs, primers, other proteins and enzymes (e.g., additional polymerases/reverse transcriptases, proteins and enzymes other than polymerases/reverse transcriptases, or both), buffering agents, or combinations thereof. Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising dNTPs may include one, two, three of all four of dATP, dTTP, dGTP and dCTP. A kit may further comprise one or more modified nucleotides. A kit may optionally comprise one or more primers (random primers, bump primers, exonuclease-resistant primers, chemically-modified primers, custom sequence primers, or combinations thereof).

A kit may be a non-natural collection of components configured, for example, for convenient storage, shipping, delivery, and/or use (e.g., use in one-pot reactions). One or more components of a kit may be included in one container for a one-pot reaction, or one or more components may be contained in one container, but separated from other components for sequential use or parallel use or controllable commencement of a desired condition or reaction. The contents of a kit may be formulated for use in a desired method or process.

A kit is provided that contains: (i) a variant Cas12a having at least 75% identity to any of SEQ ID NO: 1-16; and (ii) a buffer. The variant Cas12a may have a lyophilized form or may be included in a buffer (e.g., a storage buffer or a reaction buffer in concentrated form). A kit may contain the variant Cas12a in a mastermix suitable for receiving and amplifying a template nucleic acid. A variant Cas12a may be a purified enzyme so as to contain substantially no DNA or RNA and no other nucleases. The reaction buffer in (ii) and/or storage buffers containing the variant Cas12a in (i) may include non-ionic, ionic e.g. anionic or zwitterionic surfactants and crowding agents. A kit may optionally include a polymerase (e.g., a thermostable DNA polymerase) and/or a guide compatible with the variant Cas12a. A kit may include the variant Cas12a, the polymerase (if included), the guide (if included), and the reaction buffer in a single tube or in different tubes.

In some embodiments, a kit may include one or more oligonucleotides that bind to a predetermined nucleic acid template (e.g., one or more primers for isothermal amplification of a target nucleic acid). Example primers include isothermal amplification primers, exonuclease-resistant primers, chemically-modified primers (e.g., for fluorescence or lateral flow detection), sequencing primers, and combinations thereof. In some embodiments, a kit may exclude primers (e.g., where an end user provides primers suited for a selected target nucleic acid). In some embodiments, a kit may include a polynucleotide control (e.g., an amplification control and/or nuclease cleavage control). Examples of a control include a plasmid, a linear RNA or DNA, and a control primer (e.g., rActin control).

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Methods of Use

Isothermal amplification methods provide detection of a nucleic acid target sequence in a streamlined, exponential manner, and are not limited by the constraint of thermal cycling. Examples of isothermal amplification methods include Loop-Mediated Isothermal Amplification (LAMP), Whole Genome Amplification (WGA), Strand Displacement Amplification (SDA), Helicase-Dependent Amplification (HDA), Recombinase Polymerase Amplification (RPA), Nucleic Acid Sequences Based Amplification (NASBA). In a typical isothermal amplification reaction (e.g., LAMP, SDA, RPA, etc.), many non-specific amplification events may occur resulting in a complex array of products (e.g., multiply branched DNA products) as shown by the multiple banding pattern in the gel of FIG. 12. Results from these reactions may be difficult to interpret. The combination of isothermal amplification reaction with Cas12a detection in a one-pot reaction increases the specificity and reduces the background of nucleic acid molecular diagnostics. It further minimizes the risks of carry-over cross-contamination. To enable a bona fine one-pot detection reaction, it is desirable to utilize variants of Cas nucleases with high trans activity that are compatible with the temperature and reaction conditions of a given isothermal amplification reaction.

In some embodiments, a Cas12a variant works efficiently at temperature range between 37°−55° C. In some instances, it is desirable to couple a Cas12a to an isothermal amplification reaction whose temperature optimal is between 37°−42° C., or between 42°-50° C., or between 50°−55° C., or between 40°−55° C. For example, RPA is an example of isothermal amplification technique operating at a temperature range of 37°−42° C. For example, NASBA is an example of an isothermal amplification technique operating at a temperature range of 40°−55° C. For example, LAMP is an example of an isothermal amplification technique operating at a temperature range of 55°−65° C. According to some embodiments, a one-pot reaction maybe performed by incubating at temperatures ≥1° C., ≤20° C., ≤25° C., ≤30° C., ≤35° C., ≤40° C., ≤45° C., ≤50° C., ≤55° C., ≤60° C., and/or ≤65° C. In some embodiments, the reactions may be performed by incubating at ≥35° C., ≤40° C., ≤45° C., ≤50° C., ≤55° C., ≤60° C., and/or ≤65° C. In some embodiments, the reactions may be performed by incubating at temperatures ≥45° C., ≤50° C., ≤55° C., ≤60° C., and/or ≤65° C. A composition, in some embodiments, may have any of the foregoing temperatures.

In some embodiments, amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like.

In some embodiments dNTPs can include one, two, three of all four of dATP, dTTP, dGTP and dCTP, and can include one or more modified dNTPs, such as forms that are resistant to, or susceptible, to a particular enzymatic or chemical conversion, or that are detectable (e.g., intrinsically fluorescent nucleotides such as 1,N6-Etheno-2′-deoxyadenosine-5′-triphosphate; 3′-O—(N-Methyl-anthraniloyl)-2′-deoxyadenosine-5′-triphosphate; 2-Amino-2′-deoxyadenosine-5′-Triphosphate; 3′-O—(N-Methyl-anthraniloyl)-2′-deoxyguanosine-5′-triphosphate; 7-Deaza-7-propargylamino-2′-deoxyguanosine-5′-triphosphate; 2′,3′-O-Trinitrophenyl-cytidine-5′-triphosphate, etc.). Other examples of modified dNTPs include alpha-phosphorothioate dNTPs, dUTP, dITP, labeled dNTPs such as, e.g., fluorescein- or cyanin-dye family dNTPs. Examples herein describe inclusion of dUTP in LAMP reactions to reduce carryover contamination. Incorporation of dUTP by a DNA polymerase is commonly used during amplicon generation, and excision of incorporated uracil in copied DNA product can be catalyzed by an uracil DNA glycosidase (UDG).

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Likewise, a polymerase useful in accordance with the invention may be any specific or general polymerase known in the art and useful or the invention, including any of the polymerases and reverse transcriptases disclosed herein.

According to some embodiments, a one-pot detection method may include a reaction mixture comprising a magnesium salt (e.g., magnesium chloride) at any desired Mg²⁺ concentration (e.g., 2 mM, 6 mM, 14 mM or any other concentration 0.5 to 50 mM).

In some embodiments, a DNA polymerase for isothermal amplification is in a form selected from: dried form, lyophilized form, and solution form, wherein the solution is optionally glycerol-free. A one-pot detection reaction may include one or more other enzymes, as suitable for a particular purpose. For example, a kit for performing an RT-LAMP Cas12a one-pot reaction may optionally include a reverse transcriptase in cases where the selected DNA polymerase reverse transcriptase activity is insufficient under the selected reaction conditions.

In some embodiments, cleavage efficiency may be modulated by introduction of mismatches (e.g., ≥1 such as a mismatch or 2 between a spacer sequence and a target sequence and/or along the spacer/target. For example, the more central (e.g., away from 3′ and/or 5′ ends) a double mismatch is, the more cleavage efficiency may be affected. By choosing the mismatch position along the spacer, cleavage efficiency may be modulated. For example, if less than 100% cleavage of targets is desired (e.g. in a cell population), 1 or more mismatches (e.g., 2 mismatches) between spacer and target sequence may be introduced in the spacer sequences. The more central the mismatch position along the spacer, the lower the cleavage percentage, according to some embodiments.

Cleavage efficiency may be exploited to design single guides that distinguish two or more targets that vary by a single nucleotide (e.g., a single nucleotide polymorphism (SNP) or (point) mutation). The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets (e.g., an on-target SNP). The guide RNA may be further designed to have a synthetic mismatch. As used herein a “synthetic mismatch” refers to a non-naturally occurring mismatch that is introduced upstream or downstream of a naturally occurring SNP (e.g., ≤5 nucleotides upstream or downstream, for instance 4, 3, 2, or 1 nucleotide upstream or downstream), where closer proximity (including adjacency) of the synthetic mismatch and naturally occurring SNP may be preferred. When the CRISPR effector binds to the on-target SNP, only a single mismatch will be formed with the synthetic mismatch and the CRISPR effector will continue to be activated and a detectable signal produced. When a guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In some embodiments, a guide RNA comprises a spacer sequence with a SNP at position 1, 2, 3, 4, 5, 6, 7, 8, 9, 10, 11, 12, 13, 14, 15, 16, 17, 18, 19, or 20 of the spacer sequence (starting at the 5′ end). For example, a guide RNA may comprise a SNP at position 1, 2, 3, 4, 5, 6, 7, 8, or 9 of the spacer sequence, at position 2, 3, 4, 5, 6, or 7 of the spacer sequence or at position 3, 4, 9, or 10 of the spacer sequence, in each case, with numbering starting at the 5′ end.

According to some embodiments, a guide RNA comprises a spacer sequence with a mismatch nucleotide (e.g., a synthetic mismatch) at position 1, 2, 3, 4, 5, 6, 7, 8, 9, 10, 11, 12, 13, 14, 15, 16, 17, 18, 19, or 20 of the spacer sequence (starting at the 5′ end). For example, a guide RNA may comprise a mismatch (e.g., a synthetic mismatch) at position 1, 2, 3, 4, 5, 6, 7, 8, or 9 of the spacer sequence or at position 3, 4, 9, or 10 of the spacer sequence, in each case, with numbering starting at the 5′ end.

A guide RNA, according to some embodiments, may comprise a mismatch and a SNP wherein the mismatch is located 2 nucleotides upstream of the SNP or is located 2 nucleotides downstream of the SNP (in each case, leaving one intervening nucleotide).

In some embodiments, a one-pot detection workflow may comprise, include, and/or use any of the enzymes and compositions disclosed herein.

In some embodiments, the guide RNA of Cas12a may comprise a 20 nt spacer, 22 nt, or 24 nt spacer, which optionally, may include one or more unnatural nucleotides that increase the strength of the base pairing, such as 2-aminoadenosine (“Z”) or modified nucleotides that increase the hydrolytic stability of the guide (e.g., 2′-O-methylated nucleotides, 2′-fluoro-nucleotides, 2′-O-methoxyethyl-nucleotides, phosphorothioates nucleotides, LAN nucleotides, etc.)

According to some embodiments, an RNA target may comprise one or more modified nucleotides (e.g., anywhere in the nucleotide sequence including within the recognition sequence). Examples of modified nucleotides include N6-methyl-adenosine (m⁶A), 1-methyl-adenosine (m¹A), 5-methyl-cytidine (m⁵C), 5-hydroxymethyl-cytidine (hm⁵C),N4-acetyl-cytidine (ac⁴C), 5-methoxycytidine (mo⁵C), 4-thiouridine (SAU), 2-thiouridine (S²U), pseudouridine (Y), N¹-methyl-pseudouridine (m¹Y), 5-methyluridine (m⁵U), or 5-methoxyuridine (mo⁵U). Examples of RNA targets include RNA molecules selected from (or RNA comprising RNA molecules selected from a messenger RNA (mRNA), a ribosomal RNA (rRNA), a transfer RNA (tRNA), a small RNA (sRNA), a microRNA (miRNA), a long noncoding RNA (lncRNA), a circular RNA (circRNA), a mitochondrial RNA (mtRNA), an aptamer RNA, an antisense RNA, a silencing RNA (siRNA), or a therapeutic RNA.

In some embodiments, a method may comprise contacting a variant Cas12a having an amino acid sequence that is ≥75% identical to any of SEQ ID NOS: 1-16, a guide RNA, and an RNA target in the presence of isothermal amplification reagents.

### EXAMPLES

Some specific example embodiments may be illustrated by one or more of the examples provided herein.

### Example 1: Trans Nuclease Activity Assay

Cas12a trans nuclease activity assays were carried out in 1× modified NEBuffer r2.1 (10 mM Tris-HCl, pH 7.9 @ 25° C., 10 mM Mg²⁺, 100 μg/ml recombinant Albumin). Because Cas12a trans nuclease activity is sensitive to salt concentration in the reaction, each reaction was supplemented with NaCl to a final of 50 mM or 100 mM (specified in figure description) after accounting for the NaCl introduced from stock Cas12a, which is stored in a buffer that consists of 500 mM NaCl.

Preparation. In a trans nuclease activity assay, 100 nM Cas12a·crRNA (RNP) complex was prepared by mixing Cas12a and crRNA at a 1:1.5 molar ratio in 1× modified NEBuffer r2.1, supplemented with NaCl to final 50 mM or 100 mM, 1 mM TCEP, and incubated at room temperature for 15 minutes to form the RNP complex.

Thermal inactivation. After RNP formation, the reaction mixture was incubated at 55° C. for 5 min on a thermocycler (with lid temperature at 60° C.), then moved to ice for the rest of the steps. The elevated temperature incubation may inactivate thermolabile enzymes.

Reaction setup. After equilibration on ice for at least 2 min, 200 nM NZ-GT reporter and 2 nM of E gene PCR product E1-PCR (both pre-equilibrated on ice for at least 5 min) were added to the reaction. After rapid inversion of the tubes for at least 15 times followed by a brief spin to collect solution to the bottom, the reactions were put back on ice and aliquoted to at least 3 wells in a 96-well plate on ice.

Trans cleavage reaction. The plate was moved onto a BioRad Touch CFX96 to monitor the fluorescence. Each plate was incubated for times and at 55° C. or 37° C. as indicated in each example. Fluorescence readings by the SYBR/FAM channel (˜7 seconds each) were collected 60-100 times at 22 s intervals during the single-temperature incubation, wherein each cycle consists of ˜15 s without detection and ˜7 s plate read.

Some assays were further supplement with dNTPs. When such treatments were applied, details were specified in each figure description.

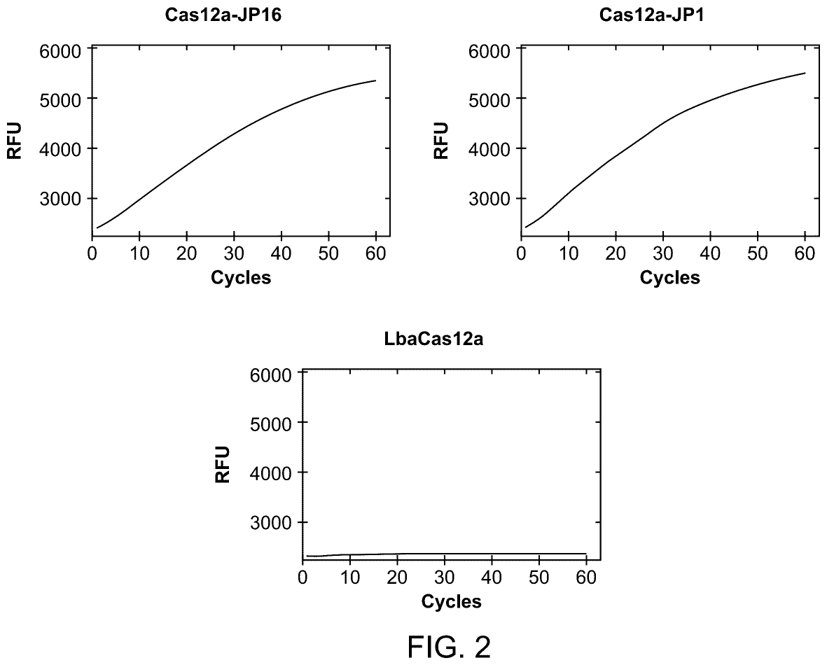

FIGS. 2, 3, 6, 9, 14, 16, 20, 21, 22, 23, 24, 26, 27, 28, 29, 30, 31, 33, 34, 35, 36, and 38 show examples of results of the trans nuclease activity (without amplification) of Cas12a variants performed in accordance with conditions describe in Example 1. The trans nuclease activity assay was performed with pre-treatment of the RNP at 55° C. for 5 min to inactivate thermolabile Cas12a enzymes. In the example of FIG. 2, Cas12a-JP16 and Cas12a-JP1 showed fluorescence due to the trans nuclease activity, whereas LbaCas12a showed no fluorescence due to heat inactivation. The trans nuclease activity assays with other Cas12a variants also showed trans nuclease activity after this heat pre-treatment, indicating that they are thermostable.

The trans nuclease activity assay was performed with limited Cas12a·crRNA·DNA cis complex (2 nM) and excess trans substrate (200 nM), so the initial rate of fluorescence change (RFU) indicates the trans nuclease efficiency. Results from these assays indicate that the trans nuclease efficiency depends on the Cas12a variant and guide choice. In the example of FIG. 3, crRNA4 was the most efficient guide out of the four guides tested with the Cas12a variant Cas12a-JP16 and crRNA2 was less efficient by comparison. When these guides are used in complex with Cas12a in one-pot detection of nucleic acid, as shown in example FIGS. 4A and 4B, crRNA4, and not crRNA2, enabled successful detection of the target RNA by Cas12a-JP16 in a one-pot reaction with RT-LAMP. In this example, higher trans nuclease efficiency is correlated with successful one-pot nucleic acid detection when coupling Cas12a with amplification.

In FIG. 6, all four crRNA guides were efficient at activating trans nuclease activity with the Cas12a variant Cas12a-JP15. FIGS. 7B-7D illustrate that the combination of crRNA2, crRNA3, or crRNA4 with Cas12a-JP15 in a one-pot reaction with RT-LAMP successfully detected target RNA. These results indicates that high trans nuclease efficiency is correlated with successful one-pot nucleic acid detection when coupling Cas12a with amplification. The failure of crRNA1 to enable one-pot nucleic acid detection (FIG. 7A), despite its high trans nuclease activity similar to crRNA3 (FIG. 6), indicates that the efficiency of crRNA as shown by a trans nuclease activity assay performed in accordance with Example 1 is not the only determinant for successful one-pot nucleic acid detection, and other factors (e.g., reaction temperature, concentration of reagents, primer design, etc.) needed to be optimized to enable one-pot detection under these conditions.

Consistent with FIG. 3, FIG. 6 and the results shown throughout the examples reveal that a Cas12a variant having higher trans nuclease activity correlates with and predicts that such variant Cas12a is more likely to enable successful detection of nucleic acid in one-pot reactions that include amplification.

### Example 2: Standalone (RT)-LAMP and (RT)-LAMP Cas12a Coupled One-Pot Reactions

To compare performance of nucleic acid detection by standalone (RT)-LAMP and (RT)-LAMP and Cas12a coupled one-pot reactions, assays were set up on ice by making a master mix that consisted of 1× LAMP primers (1.6 uM FIP and BIP (e.g. SEQ ID NO:43, 44, 57, 58, 63, and 64), 0.4 uM LF and LB (e.g. SEQ ID NO:45, 46, 59, 60, 65, and 66), 0.2 uM F3 and B3 (e.g. SEQ ID NO:41, 42, 55, 56, 61, and 62), 1 mM each dNTPs, 1 uM NZ-GT reporter (SEQ ID NO:37), 1 uM SYTO™ 82 (dsDNA binding dye, Thermo Fisher S11363), 0.3 U/μl WarmStart RTx Reverse Transcriptase (New England Biolabs, M0380S), and 0.32 U/μl BD009-SDpol-1. in a reaction buffer that consists of 50 mM Tris-HCl, pH 8.5 at 25° C., 75 mM KCl, 7 mM MgSO₄, and 0.05% Tween 20. The master mix was aliquoted to PCR tubes.

For the one-pot reactions, the aliquoted mixture was supplemented with synthetic SARS-COV-2 RNA (Control 2, Twist Biosciences) or equivalent water (as no-template control, or NTC), and 100 nM of Cas12a RNP. The Cas12a RNP was prepared by mixing Cas12a and crRNA at 1:1.5 molar ratio in 1× NEBuffer r2.1 supplemented with 1 mM TCEP, and incubated at room temperature for 15 minutes before addition to one-pot reaction. The reverse transcriptase was included in RT-LAMP and RT-LAMP Cas12a coupled one-pot reactions, and not in LAMP and LAMP Cas12a coupled one-pot reactions. For the standalone (RT)-LAMP reactions, the Cas12a RNP was substituted with an equal amount of water. Each tube was then inverted rapidly at least 20 times to ensure through mixing, and after a brief spin to collect the liquid, the samples were returned to ice. Each reaction mixture was then aliquoted to 96-well plate by multi-channel pipette for at least 2× repeats. The 96-well plate was transferred onto a BioRad CFX96 touch, which was preheated to 55° C. Reactions were incubated at 55° C. for 30 s, followed by 60-100 cycles of 30-s incubation at 55° C. followed by plate read (monitoring All Channels). Thus, each cycle is approximately 42 s (30 s plus 12 s plate read). Variations to this standard protocol were specified in in each figure description.

FIGS. 4A, 4B, 5, 7A-7D, 8, 10A, 10B, 25A, 25B, 32A, 32B, 37A-D, 39A, 39B, 40A-40E, 41, 42A-42D, 43A, and 43B show examples of results from reactions performed in accordance with the conditions described in Example 2. The fluorescence in one-pot reactions corresponds to FAM signal generated by released FAM fluorophore due to the Cas12a trans nuclease activity on the reporter. Presence of the FAM signal indicates presence of the target, and absence of FAM signal indicates absence of the target. The presence of FAM fluorescence only in positive samples indicates that the one-pot nucleic detection method is specific. The fluorescence in standalone (RT)-LAMP reaction corresponds to HEX signal generated from intercalating dye SYTO™ 82 binding to dsDNA from amplification reaction.

In the example of FIG. 4A, the absence of FAM fluorescence (background level) both with the positive sample and NTC indicates that the guide crRNA2 failed to enable Cas12-JP16 in detecting target RNA. In the example of FIG. 4B, FAM fluorescence was only observed for the positive sample and not in the NTC, indicating that Cas12a-JP16 complexed with guide crRNA4 could specifically detect target RNA in the sample. FIG. 5 shows HEX fluorescence signal in both positive and NTC reactions, even though there was a delay in NTC. Comparison of the results in FIG. 4B and FIG. 5 demonstrates that detection of target RNA by coupling RT-LAMP with Cas12a (relying on the signal from Cas12a reaction; FAM signal in this case) is more specific and easier to interpret than that of the standalone RT-LAMP (relying on signal from LAMP reaction; HEX signal in this case). Collectively, the results of these examples show that detection of nucleic acid by coupling Cas12a and (RT)-LAMP is more specific and easier to interpret the results than standalone (RT)-LAMP, and this applies with the additional examples of Cas12a variants deployed for one-pot Cas12a-coupled (RT)-LAMP reactions in accordance with Example 2, as described in FIGS. 7A-7D, 8, 10A, 10B, 25A, 25B, 32A, 32B, 39A, 39B, 40A-40E, 41, 42A-42D, 43A, and 43B.

### Example 3: Standalone SDA and SDA Cas12a Coupled One-Pot Reactions

To compare performance of nucleic acid detection by standalone SDA and SDA Cas12a coupled one-pot reactions, assays were set up on ice by making a master mix that consisted of 0.4 mM each dNTPs, 0.5 uM of each primers (e.g. SEQ ID NO:67, 68, 69, and 70), 1 uM NZ-GT reporter (SEQ ID NO:37), 1 uM SYTO™-82, 0.4 U/μl WarmStart Nt.BstNBI (NEB R0725S), and 0.3 U/μl BD009-SDpol-1 in a reaction buffer that consists of 1× standard LAMP buffer (20 mM Tris-HCl, pH 8.8 at 25° C., 50 mM KCl, 10 mM (NH₄)₂SO₄, 2 mM MgSO₄, and 0.1% Tween 20). The master mix was then aliquoted to PCR tubes.

For the one-pot reactions, the aliquoted mixture was supplemented with 10 pM E1 gene PCR product (E1-PCR) as the nucleic acid input (positive) or equivalent water (NTC), and 100 nM Cas12a RNP. The Cas12a RNP was prepared by mixing Cas12a and crRNA at 1:1.5 molar ratio in 1× NEBuffer r2.1 supplemented with 1 mM TCEP, and incubated at room temperature for 15 minutes before addition to one-pot reaction. For the standalone SDA reactions, everything was the same as one-pot reactions except that the Cas12a RNP was substituted with equal amount of water. Each tube was inverted rapidly at least 20 times to ensure through mixing, and after a brief spin to collect the liquid, the samples were returned to ice. Each reaction mixture was then aliquoted to 96-well plate by multi-channel pipette for at least 3× repeats. The 96-well plate was then transferred onto a BioRad CFX96 touch, which was preheated to 58° C. The reactions were incubated at 58° C. for 30 s, followed by 60-100 cycles of incubation at 58° C. (for 30 s followed by plate read monitoring All Channels). Thus, each cycle is approximately 42 s (30 s plus 12 s plate read). Variations to this standard protocol were specified in in each figure description.

FIGS. 11A 11B, 12, 13A, 13B, 15A and 15B show example results of reactions performed in accordance with the conditions described in Example 3. The fluorescence in one-pot reactions corresponds to FAM signal generated by released FAM fluorophore due to the Cas12a trans nuclease activity on the reporter. Presence of the FAM signal indicates presence of the target, and absence of FAM signal indicates absence of the target. The fluorescence in standalone SDA reaction corresponds to HEX signal generated from intercalation of the dye SYTO™ 82 to dsDNA generated from the SDA amplification reaction. Presence of HEX signal indicates generation of DNA by amplification.

Results in FIGS. 11A and 11B show comparison of nucleic acid detection by one-pot reaction coupling Cas12a with SDA (FIG. 11A) or standalone SDA (FIG. 11B). The one-pot reaction shows presence and absence of FAM fluorescence with the positive sample and negative control (NTC), respectively. FIG. 11B (standalone SDA) shows HEX fluorescence in both positive and NTC reactions (fluorescence signal is delayed in the NTC reaction). The results in FIGS. 11A and 11B indicate that the detection of target by coupling SDA with Cas12a is more specific and easier to interpret than by standalone SDA.

FIG. 12 shows analysis of the SDA product by gel electrophoresis. The three reaction repeats with positive sample input showed a distinct band that fits the expected amplicon size (labeled by asterisks) whereas the NTC showed a light smear (from nonspecific amplification). Note that other bands are also observed in the positive samples due to nonspecific amplifications. The gel electrophoresis confirms the presence of target nucleic acid in the positive samples and absence of target in the NTC, but this process is more labor-intensive and runs the risk of contaminating the working environment due to the necessity of opening of the reaction vessel. In contrast, nucleic acid detection by one-pot method in a Cas12a-coupled SDA reaction, as shown in FIG. 11A, is easy to interpret and specific (i.e., presence or absence of a signal), and poses no cross-contamination risk as once the one-pot assay is set up, the reaction vessel does not need to be opened for result analysis.

Additional examples of Cas12a variants deployed for one-pot Cas12a-coupled SDA reactions are described in FIGS. 13A, 13B, 15A and 15B. In all cases, one-pot reaction coupling Cas12a with SDA demonstrates specific detection of target with easy setup and no cross-contamination risk for result interpretation.

### Example 4: Standalone RPA and RPA Cas12a Coupled One-Pot Reactions

To compare performance of nucleic acid detection by standalone RPA and RPA Cas12a coupled one-pot reactions, assays were all set up on ice. Each reaction consisted of 0.5 uM each of the primers (e.g. SEQ ID NO:67 and 70), 1 uM T15 reporter (SEQ ID NO: 38), 1 mM TCEP, 29.5 ul of TwistAmp® Basic (TwistDx TABAS03KIT) by rehydrating one well of the reagent with 29.5 ul of water at room temperature, and 0.4 pM E1 gene PCR product (E1-PCR) as the nucleic acid input (positive) or equivalent water (NTC). For the RPA-Cas one-pot reactions, each reaction was supplemented with 100 nM Cas12a RNP or equivalent water (standalone RPA). The Cas12a RNP was prepared by mixing Cas12a and crRNA at 1:1.5 molar ratio in 1× NEBuffer r2.1 supplemented with 1 mM TCEP, and incubated at room temperature for 15 minutes before addition to one-pot reaction. Reactions were started by addition of 14 mM MgOAc in a total reaction volume of 50 ul. Reaction mixtures were inverted rapidly at least 20 times to ensure through mixing, then aliquoted to a 96-well plate by multi-channel pipette for at least 3× repeats for each reaction. The 96-well plate was then transferred onto a BioRad CFX96 touch, which was preheated to 42° C. The reactions were incubated at 42° C. for 60-100 cycles of incubation for 30 s followed by plate read monitoring All Channels). Thus, each cycle is approximately 42 s (30 s plus 12 s plate read). Variations to this standard protocol were specified in in each figure description.

FIGS. 17A, 17B, 18, 19A, and 19B show example results of reactions performed in accordance with the conditions described in Example 4. The fluorescence in one-pot reactions corresponds to FAM signal generated from cleavage of the reporter by the Cas12a. Presence of the FAM signal indicates presence of the target, and the absence of the FAM signal indicates absence of the target. The fluorescence in standalone RPA reaction is background FAM signal without the cleavage of the reporter.

Results in FIG. 17A show FAM fluorescence signal with the positive sample (presence of target) and background FAM signal in the NTC (absence of target) in one-pot reactions coupling RPA and Cas12a. FIG. 17B shows background FAM signal in the standalone RPA reactions with both the positive sample and NTC, requiring additional analysis step to differentiate the two (such as by gel electrophoresis shown in FIG. 18). The results in FIGS. 17A and 17B indicate that detection of nucleic acid by fluorescence via coupling RPA with Cas12a is specific and straightforward.

Results in FIG. 18 show analysis of the RPA product by gel electrophoresis. The reaction products in FIG. 17B were loaded on a 3% agarose gel and separated by electrophoresis. The DNA amplification products bind to the SYBR green dye. The gel was visualized under UV. The reaction with positive sample input showed a distinct band that fits the expected amplicon size (labeled by an asterisk) whereas the NTC showed light smear. The gel electrophoresis confirmed the presence of the target in the positive sample and absence in the NTC, but this process is more labor-intensive and risks contaminating the testing space due to the opening of the RPA reaction and aerosolization of the DNA product. In contrast, nucleic acid detection by one-pot method coupling RPA with Cas12a, as shown in FIG. 17A is easy to set up, simple to interpret the results, and poses no cross-contamination risk as once the one-pot assay is set up there is no need to open the reaction vessel for analysis.

Additional examples of Cas12a variants deployed for RPA Cas12a coupled one-pot reactions are described in FIGS. 19A and 19B. Similarly, presence and absence of the target is correlated with presence and absence of the fluorescence signal, respectively, in the one-pot reaction shown in FIG. 19A. The standalone reaction show in FIG. 19B requires further analyses (such as gel electrophoresis) to interpret the results. Consistently, these results demonstrate that one-pot reactions coupling Cas12a with RPA for nucleic acid detection is simple and straightforward compared to standalone RPA.

### Example 5: Single Nucleotide Polymorphism (SNP) Detection by Cas12a (RT)-LAMP Coupled One-Pot Reaction

Cas12a guide spacer with one or more single nucleotide polymorphisms (SNPs) to the target could affect the Cas12a trans nuclease activity (see, e.g. Fuchs et al. Commun Biol 2022. https://doi.org/10.1038/s42003-022-03275-2). This feature can be leveraged to design guides that can distinguish two or more targets that vary by one or more nucleotides, or SNPs. Reactions for SNP detection by Cas12a (RT)-LAMP coupled one-pot reaction were set up following the same reaction condition in accordance with Example 2, except that different guide RNAs, either with perfect match to the target or with mismatches to the target, were used to for Cas12a-crRNA RNP complex in the reactions. Details that are not in accordance with Example 2 are specified in the figure description.

FIGS. 40A-40E, 42C and 42D, and 43A and 43B show example results of one-pot reactions that couple (RT)-LAMP with Cas12a-JP15 using guide RNAs that harbor various number of SNPs.

For example, the impact of single mismatch on nucleic acid detection by one-pot reaction coupling RT-LAMP with Cas12a was tested and shown in FIGS. 40A-40C. Compared with reactions with perfect match guide crRNA4 (FIG. 40A), reaction with crRNA harboring a C to G change (generating a G: G mismatch between the guide and target) at position 9 showed reduced signal level (final RFU, FIG. 40B) with the same detection time. Compared with reactions with perfect match guide crRNA4 (FIG. 40A), reaction with crRNA harboring an A to U change (generating a U: T mismatch between the guide and target) at position 15 showed similar results (FIG. 40C), i.e., same signal level in terms of the final RFU and detection time. These results indicate that single mismatches between the guide and target may reduce one-pot nucleic acid detection efficiency depending on its position. For example, mismatch at position 9 seems to have a bigger impact on Cas12a-JP15 activity and eventual one-pot detection efficiency than mismatch at position 15.

For example, the impact of double mismatches on nucleic acid detection by one-pot reaction coupling RT-LAMP with Cas12a was tested and shown in FIGS. 40D-40E. Compared with reactions with perfect match guide crRNA4 (FIG. 40A), reaction with crRNA harboring a C to G change at position 9 and U to A change at position 10 (generating G: G and A: A double mismatches between the guide and target) reduced the signal level to background level (FIG. 40D). Compared with reactions with perfect match guide crRNA4 (FIG. 40A), reaction with crRNA harboring a G to C change at position 3 and G to C change at position 4 (generating C: C and C: C double mismatches between the guide and target) reduced the signal level significantly, but slightly higher than background level (FIG. 40E). These results indicate that consecutive double mismatches between the guide and target may reduce one-pot nucleic acid detection efficiency significantly or even eliminate the signal depending on the positions. For example, mismatches at positions 9 and 10 seem to have a bigger impact on Cas12a-JP15 activity and eventual one-pot detection efficiency than mismatches at positions 3 and 4. Based on these results, if two targets have a single nucleotide difference (SNP), it is reasonable to predict that, guides that are designed to either perfectly match one of the targets (the guide would have a single mismatch to the other target), or with mismatches at positions 9 and 10 to one of the targets could result in presence or absence of the reporter signal to differentiate the targets with a single SNP.

For example, in FIGS. 43A and 43B, the Mpox_crRNA1-9c10c spacer (UGUGCAAUCCUUGGACUUUG; e.g., nucleotides 21-40 of SEQ ID NO:31) has two mismatches to the target region of Mpox (TGTGCAATAATTGGACTTTG; e.g., nucleotides 310-329 of SEQ ID NO:47) on the 9^{th} and 10^{th} positions (underlined), and a single mismatch on the 10^{th} position compared to Var target region (TGTGCAATCATTGGACTTTG; e.g., nucleotides 310-329 of SEQ ID NO:48). In the one-pot assay with this guide, there was prominent FAM fluorescence with Var target but background FAM fluorescence with Mpox target, clearly differentiating these two targets. These results indicate that proper design of mismatch(es) on the guide can be used to distinguish targets with SNPs in one-pot nucleic acid detection reactions coupling Cas12a with amplification.

### Example 6: Compatibility of Carryover Prevention Procedure with One-Pot Nucleic Acid Detection Coupling Cas12a with Amplification

Repeated nucleic acid tests using amplification methods, such as isothermal amplification, can generate aerosolized amplification products and contamination of the work environment. This can lead to carryover contamination of tests and false-positive results. Addition of dUTP to the dNTPs mix, in combination with uracil-DNA glycosylase (UDG), has been applied in carryover prevention procedures in standalone (RT)-LAMP for nucleic acid detection (see e.g., Hsich K, et al. Chem Commun (Camb). 2014 Apr. 11; 50 (28): 3747-9.). Thus, it is important to test if this common procedure is compatible with one-pot nucleic acid detection coupling Cas12a with (RT)-LAMP.

One-pot reactions with the inclusion of dUTP were set up following the same reaction condition in accordance with Example 2, except that different amounts of dUTP, instead of the standard 1 mM dNTPs, were used in the reaction. Details that are not in accordance with Example 2 are specified in the figure description. FIGS. 37A-37D show examples of results from one-pot reactions performed with the inclusion of dUTP.

In the example of FIGS. 37A-37D, one-pot reactions coupling Cas12a and RT-LAMP were performed with different amounts of dUTP instead of the standard 1 mM dNTPs in the reaction. The reactions with 0% dUTP (37A) were supplied with 1 mM of each dNTPs, i.e., dATP, dCTP, dGTP, and dTTP, and no dUTP. The reactions with 50% dUTP (37B) were supplied with 0.5 mM dUTP in addition to 1 mM of each of the dATP, dCTP, dGTP, and dTTP. The reactions with 100% dUTP (37C) were supplied with 1 mM of each of the dATP, dCTP, dGTP, and dUTP. Regardless of the dUTP amount incorporated in the reaction, the one-pot reactions all showed prominent fluorescence signal in the presence of the target and background fluorescence in the absence of the target. The time of detection of the target, as shown by FIG. 37D was indistinguishable for 0% (circle) and 50% dUTP (triangle), and slightly delayed for 100% dUTP (diamond). The condition of 50% dUTP, i.e. dNTPs with the addition of 0.5× molar ratio of dUTP, was usually applied in carry-over prevention procedures in LAMP in combination with uracil-DNA glycosylase (UDG). Thus, these results indicate that the one-pot nucleic acid detection method coupling LAMP with Cas12a could be compatible with carry-over prevention practices, such as the example shown here with the combination of dUTP.

In the example of FIGS. 38, the impact of the presence of dU in the DNA target on the trans nuclease activity was tested directly. The target E gene DNA (E1-PCR) was prepared by amplification with OneTaq DNA polymerase (NEB #M0480) following the recommended protocol of the product, except that the 10 mM dNTPs mix was substituted with a dNTPs mixture with 0% dUTP, 50%, or 100% dUTP from individual dATP, dCTP, dGTP, dTTP, and dUTP. The definition of the % dUTP conditions is the same as aforementioned for FIGS. 37A-37D. The PCR product was cleaned up by standard spin column and quantified by nanodrop. To minimize the errors in quantification of DNA, the trans nuclease activity assay was performed with limiting RNP and excess cis substrate. This way the same amount of Cas12a-crRNA-cis DNA product complex would react with excess reporter to test the effect of dU-incorporation in DNA on trans nuclease activity. Specifically, the Cas12a-JP15 enzymes (15 nM) was loaded with 1.5× crRNA4 for E gene of SARS-COV-2 to form an RNP complex, which was activated by 20 nM cis substrate E gene DNA (E1-PCR with different dU incorporation). The trans nuclease activity was monitored by FAM signal from cleavage of the NZ-GT reporter, which was included to a final concentration of 300 nM. Reactions were carried out in 1× NEBuffer r2.1, supplemented with 1 mM dNTPs, 1 mM TCEP, and incubated at 55° C.

Example FIG. 38 showed that the presence of dU in the cis DNA does not affect the Cas12a-JP15 trans nuclease efficiency under the conditions tested, as estimated by the rate of RFU increase in the initial linear phase. These results provide a mechanistic basis for the compatibility of dUTP with one-pot nucleic acid detection methods coupling amplification with Cas12a.

### Example 7: Determination of Protospacer Adjacent Motif (PAM) Recognition Pattern

Cas12a nucleases recognizes target by its guide RNA spacer (˜20 nt) complementarity to the target DNA sequence, as well as nucleotide and amino acid interactions adjacent to the target sequence. This recognition pattern, or protospacer adjacent motif (PAM), usually T-rich for Cas12a nucleases, is important in rapid target search, but can also restrict the targeting scope due to the requirement of the specific PAM. Thus, it is important to know the PAM requirement of each Cas12a nuclease to design guide RNA for its target. PAM recognition pattern of the example Cas12a variants described herein were determined by following methods descried in Fuchs et al. Commun Biol 2022. https://doi.org/10.1038/s42003-022-03275-2. Briefly, a circular dsDNA (120-124 bp) library with a randomized 10-nt region 5′ of the target sequence was prepared and cleaved by Cas12a RNP to determine PAM preference. Cas12a RNPs were formed by incubating 2 pmol of guide RNA with 1 pmol of Cas12a protein in 1× NEBuffer™ 2.1 at room temperature for 10 min. RNPs were added to 0.2 pmol of circular DNA substrate and incubated for 30 min at 55° C. Reactions were then quenched by the addition of 0.04 units Proteinase K and EDTA at a final concentration of 32 mM. To assess the composition of the randomized PAM region, a control reaction was performed by digesting the circular DNA library with BstXI for 30 min at 37° C. Both reactions were then purified and prepared into libraries for Illumina sequencing. The nucleotides that allow recognition and cleavage by the Cas12a were hence enriched to allow the determination of PAM preference. The PAM cleavage site was determined using custom scripts and used the following position weight equation to determine the enrichment or depletion of each base at each position in the randomized region:

$$score = {log}_{2} ( Cas 12 a sample frequency / BstXI sample frequency )$$

Results of PAM sites determined for example Cas12a variants were shown in TABLE 3.

TABLE 3

| | PAM |
|---|---|
| | (N = A, G, T, or |
| Cas12a variants | C; Y = T or C) |
| Cas12a-JP15 | YYN |
| Cas12a-JP13 | YNN |
| Cas12a-JP16 | YYN |
| Cas12a-JP19 | YYN |
| Cas12a-JP29 | YYN |
| Cas12a-JP31 | YYN |
| Cas12a-JP53 | TTN |

### Example 8: Thermostability Assay of Cas12a

Thermostability of the Cas12a nucleases was measured by two methods. One is protein unfolding using nano differential scanning fluorometry (NanoDSF) and the other one is trans nuclease activity assay after heat pre-treatment.

For thermostability test with NanoDSF, both the apo protein and the Cas12a·crRNA (RNP) complex was analyzed. The Cas12a·crRNA (RNP) complex was prepared by mixing Cas12a and crRNA4 of SARS-COV-2 E gene at 1:1.5 molar ratio for a final 10 uM RNP in 1× NEBuffer r2.1 supplemented with 1 mM TCEP. The apo Cas12a nuclease was diluted to 10 uM with 1× NEBuffer r2.1 supplemented with 1 mM TCEP. Both RNP and apo Cas12a samples were incubated at room temperature for 15 minutes before each sample was loaded into a standard 10 ul capillary for measurements. Fluorescence was monitored as temperature increased at a rate of 1° C. sec-1 over a temperature range from 20° C. to 80° C. The inflection point reflects unfolding of the protein. TABLE 4 show example results of thermostability analysis by nanoDSF performed in accordance with the condition described in Example 8. Both the apo enzyme and the RNP complex of example Cas12a variants Cas12a-JP16 and Cas12a-JP1 showed denaturing temperature higher than 55° C., similar to the wild type thermostable YmeCas12a. These results indicate that the Cas12a variants are thermostable.

For thermostability test with heat pre-treatment, trans nuclease activity of Cas12a variants were performed in accordance with conditions describe in Example 1. Specifically, after RNP formation following description in Example 1, the RNP was incubated at 55° C. for 5 min. Thermolabile enzyme, such as LbaCas12a, is inactivated by this pre-treatment, as shown in FIG. 2, by the observation of only background fluorescence in the trans nuclease activity assay. However, Cas12a-JP16 and Cas12a-JP1 showed fluorescence due to the trans nuclease 5 activity, indicating that they are thermostable. FIGS. 3, 6, 9, 14, 16, 20, 21, 22, 23, 24, 26, 27, 28, 29, 30, 31, 33, 34, 35, 36, and 38 show examples results of the trans nuclease activity of Cas12a variants performed in accordance with conditions describe in Example 1. The trans nuclease activity test of the Cas12a variants were performed with pre-treatment of the RNP at 55° C. for 5 min. The assays with these Cas12a variants also showed trans nuclease activity 10 after this heat pre-treatment, indicating that they are thermostable.

TABLE 4

Inflection point (° C.)

| Cas12a | Apo enzyme | Cas12a-crRNA RNP |
|---|---|---|
| YmeCas12a | 64.1 | 66.2 |
| Cas12a-JP16 | 57.5 | 63.1 |
| Cas12a-JP1 | 62.0 | 67.2 |

### Example 9: Construct, Expression and Purification of Cas12a Variants

15 Cas12a variants described in this invention were engineered from Yellowstone metagenome (Yme) Cas12a according to standard molecular biology methods or selected from ancestral sequences following standard ancestral sequence reconstruct procedures. E. coli codon-optimized DNA sequences encoding the Cas12a proteins were cloned into plasmids with an His₁₄ tag, maltose-binding protein (MBP), and a SenP1 cleavage site in order at the N terminal end of the Cas12a sequence to facilitate overexpression and purification. The recombinant protein was expressed in E. coli NiCo21 (DE3) cells (NEB #C2925) in LB media containing Kanamycin (40 μg/ml) at 30° C. until the growth reached the mid-exponential phase at which time IPTG was added to a final concentration of 0.4 mM and the temperature was shifted to 16° C. for 16 hr. Cells were harvested and disrupted by sonication prior to chromatographic purification. Most of the Cas12a nucleases used for trans nuclease activity assay as described in Example 1 were purified by immobilized metal affinity chromatography (IMAC) with Ni-NTA magnetic beads followed by SUMO protease cleavage of the tag, and removal of the tag by reverse IMAC. The flowthrough was collected and concentrated in storage buffer (20 mM Tris-HCl (pH 7.4), 500 mM NaCl, 1 mM DTT, 0.1 mM EDTA, 50% glycerol (v/v)) by buffer exchange. Proteins were stored at −20° C. until use. Recombinant proteins used in one-pot assays, and some for trans nuclease activity assay as described in Example 1, were purified in a larger scale and followed a slightly more complex purification procedure. The cell lysate was cleaned up first by HiTrap DEAE and the flowthrough was applied to IMAC with Ni-NTA beads. The eluted protein was treated with SUMO protease overnight at 4° C. to cleave the His14 tag along with the MBP. After reverse IMAC, the flowthrough was applied onto an ÄKTA HiTrap Heparin columns (Cytiva). Fractions were pooled prior to a final chromatographic separation using a HiLoad 16/600 Superdex 200 pg column (Cytiva). Fractions were pooled, dialyzed and concentrated into storage buffer prior to storage at −20° C. until use.

What is claimed is:

1. A composition comprising:
a variant Cas12a having an amino acid sequence at least 90% identical to one or more of SEQ ID NOS: 1-16, wherein the variant Cas12a has thermostable cis recognition activity and thermostable trans nuclease activity;
a crRNA operable with the variant Cas12a; and
optionally, a non-naturally occurring buffer.

2. A composition according to claim 1 further comprising a trans nuclease substrate.

3. A composition according to claim 1, wherein the trans nuclease substrate comprises a fluorophore, a linker polynucleotide, and a quencher, wherein the fluorophore and the quencher are each operably linked to the linker polynucleotide and wherein the quencher is operable to quench the fluorophore.

4. A composition according to claim 3, wherein the linker polynucleotide is disposed between the fluorophore and quencher and is susceptible to trans nuclease cleavage.

5. A composition according to claim 1, wherein the molar ratio of variant Cas12a: crRNA is 1:1 to 1:20.

6. A composition according to claim 1, wherein the amino acid sequence is at least 95% identical to one or more of SEQ ID NOS: 1-16.

7. A composition according to claim 1, wherein the composition is cell-free.

8. A composition according to claim 1, wherein the composition has a dried, freeze dried, lyophilized, crystalline, or aqueous form.

9. A composition according to claim 1, wherein the variant Cas12a is immobilized on a support with or without a linker.

10. A composition according to claim 1 further comprising a DNA polymerase, a glycosylase, a nicking enzyme, a ligase, a helicase, a recombinase, a crowding agent, a DNA binding protein, a dye, an additive, a ribonucleoprotein, and/or combinations thereof.

11. A composition according to claim 1 further comprising a Bst DNA polymerase, Bsu DNA polymerase, phi29 DNA polymerase, phi29-XT DNA polymerase, and/or variants thereof.

12. A composition according to claim 1 further comprising a uracil DNA glycosylase.

13. A one-pot method comprising contacting at a single temperature and in a single container:
a variant Cas12a having an amino acid sequence at least 90% identical to one or more of SEQ ID NOS: 1-16, wherein the variant Cas12a has thermostable cis recognition activity and thermostable trans nuclease activity;
a crRNA operable with the variant Cas12a, wherein the molar ratio of variant Cas12a:
crRNA is optionally 1:1 to 1:20;
a polynucleotide comprising a nucleic acid sequence of interest;
amplification primers complementary to at least a portion of the sequence of interest and operable to support amplification of the sequence of interest;
optionally, a reverse transcriptase;
a DNA polymerase;
a trans nuclease substrate; and
optionally, a non-naturally occurring buffer.

14. A method according to claim 13, wherein the variant Cas12a and the crRNA are contacted in a single container under conditions that permit loop mediated amplification of the sequence of interest and trans nuclease cleavage of the trans nuclease substrate to produce a detectable trans nuclease cleavage marker.

15. A method according to claim 13, wherein the method is performed at a single temperature in a range of 50-70° C., 55-65° C., or 50-60° C.

16. A method according to claim 13, wherein the amplification primers and the polynucleotide comprising the nucleic acid sequence of interest hybridize to form an amplification substrate for the DNA polymerase.

17. A method according to claim 14 further comprising amplifying, by the DNA polymerase, the amplification substrate to produce an amplification product.

18. A method according to claim 17, wherein the amplifying is selected from genome exponential amplification reaction (GEAR), helicase-dependent amplification (HDA), loop-mediated isothermal amplification (LAMP), multiple displacement amplification (MDA), nicking enzyme amplification reaction (NEAR), nucleic acid sequence-based amplification (NASBA), ramification (RAM), recombinase polymerase amplification (RPA), rolling circle amplification (RCA), self-sustained sequence replication (3SR), single primer isothermal amplification (SPIA), strand displacement amplification (SDA), transcription mediated amplification (TMA), or combinations thereof.

19. A method according to claim 17, wherein the crRNA hybridizes to the polynucleotide comprising the nucleic acid sequence of interest, the amplification product, or combinations thereof to form a cleavage assembly comprising the crRNA, the polynucleotide, and the variant Cas12a.

20. A method according to claim 19, wherein the variant Cas12a cleaves the polynucleotide comprising the nucleic acid sequence of interest.

21. A method according to claim 20, wherein the variant Cas12a cleaves the trans nuclease substrate to form a trans nuclease cleavage product.

22. A method according to claim 21 further comprising detecting the trans nuclease cleavage product.

23. A method according to claim 22, wherein detecting the trans nuclease cleavage product comprises optically detecting the trans nuclease cleavage product and/or electrochemically detecting the trans nuclease cleavage product.

24. A method according to claim 13, wherein the molar ratio of variant Cas12a: crRNA is 1:1 to 1:20.

25. A variant Cas12a having an amino acid sequence at least 90% identical to one or more of SEQ ID NOS: 1-16, wherein the variant Cas12a has thermostable cis recognition activity and thermostable trans nuclease activity.
